(19) United States
(12) Patent Application Publication
(10) Pub. No.: US 2023/0200362 A1
(43) Pub. Date: Jun. 29, 2023

(54) MODEL ORGANISMS HUMANIZED FOR DRUG DISCOVERY AND SCREENING

(71) Applicant: NemaMetrix, Inc, Eugene, OR (US)

(72) Inventors: Trisha J. Brock, Eugene, OR (US); Christopher E. Hopkins, Salt Lake City, UT (US); Kathryn McCormick, Eugene, OR (US)

(73) Assignee: NemaMetrix, Inc, Eugene, OR (US)

(21) Appl. No.: 17/787,899

(22) PCT Filed: Dec. 21, 2020
(86) PCT No.: PCT/US2020/066461

§ 371 Date: Jun. 21, 2022

Related U.S. Application Data
(60) Provisional application No. 62/952,218, filed on Dec. 21, 2019.

(51) Int. Cl. A01K 67/027; G01N 33/50

(52) U.S. Cl. CPC A01K 67/0275; G01N 33/5082; A01K 2217/056; A01K 2227/703; A01K 2267/03; A01K 2267/0393

(57) ABSTRACT

This disclosure relates to methods for screening therapeutic agents to treat altered function of a mutated target gene (e.g., clinical variant) as well as reagents for use in the same.

### RELATED APPLICATIONS

This application claims priority to provisional application U.S. Ser. No. 62/952,218 filed 21 Dec. 2019, which is hereby incorporated into this application in entirety.

### SEQUENCE LISTING

The instant application contains a Sequence Listing which has been submitted electronically in ASCII format via EFS-Web and hereby incorporated by reference in its entirety. Said ASCII copy, created on 21 Dec. 2020, is named NEMA011PCT_ST25.TXT and is 24,576 bytes in size.

### STATEMENT REGARDING FEDERALLY SPONSORED RESEARCH OR DEVELOPMENT

This invention was made with government support under R43AG061978 and R43NS108847 awarded by National Institute of Health (NIH). The government has certain rights in the invention.

### FIELD OF THE DISCLOSURE

This application pertains generally to transgenic model organisms, such as nematodes or zebrafish, comprising one or more chimeric heterologous genes that encode a drug target wherein the drug target effects the physical properties and/or function of the organism (e.g. phenotype) providing a method of screening the drug effectiveness.

### BACKGROUND INFORMATION

Many groups have turned to using simple animals, such as Drosophila, zebrafish and C. elegans, to model disease biology. For instance, the Undiagnosed Disease Network (UDN) has made a concerted effort to efficiently model and provide therapeutic insight into rare disease biology using cost-effective small-animal-model approaches (Reuter 2018). In many instances, modeling in simple animal systems has provided important insights into disease mechanism (Bend 2016, Wangler 2017, Luo 2017, Chao 2017, Marcogliese 2018, Oláhová 2018, Liu 2018, Guiberson 2018). The capacity to understand mechanism is due to a surprisingly high conservation of biology — 80% of disease genes have a homolog in the nematode. Traditionally, the main driver behind the use of animal models in drug screening has been their utility in measuring drug safety before clinical trials (Denayer 2014). Although effective for removing leads with undesirable effects, the use of animals only in late stage of drug development contributes to the high expense of attrition occurring at late stages of drug development. Deploying animal models earlier in the pipeline would be more efficient but would be expensive if it were to rely solely on the use of rodent models. The C. elegans nematode and Danio rerio zebrafish as an alternative model are a good choice for early deployment. They allow an affordable “fail early, fail fast” system to occur at the discovery phase of drug development where costs can be better contained.

For mice, creation and verification of homozygous mice can take a year or more (Hall 2009), yet the generation and characterization of transgenic C. elegans is much shorter and can occur in as soon as 3 days. Zebrafish is intermediate at about 3 months, and a useful model for screening therapeutic agents. The C. elegans animal model has a proven capacity for high-throughput drug screening. For instance, the Cloe Lab uses 1536-well plates on a C. elegans model to achieve the screening of 364,000 compounds in 5 weeks (Leung 2013). Recently, other labs have been achieving similar high-throughput screens in C. elegans (Rangaraju 2015, O’Reilly 2016, Lucanic 2018, Partridge 2018). C. elegans animals have other unique properties making them ideal for high-throughput methods. A fast life-cycle allows population amplification from founder to thousands of individuals in less than one week. Assays can be done on large populations shortly after transgenic isolation and verification. The C. elegans nematode has a self-fertilizing hermaphrodite lifestyle which renders animal husbandry quite simple and results in populations with low genetic heterogeneity. Finally, a capacity to thrive in liquid culture generates populations amenable to robotic liquid-handling procedures. As a result, the C. elegans nematode has become the alternative animal model of choice that is innately empowered for rapid drug screening.

Advantages of using C. elegans in high throughput drug discovery screening include: 1) the ability to model complex human diseases that cannot be easily reproduced in vitro or in unicellular models, 2) the ability to simultaneously evaluate drug efficacy and absorption, distribution, metabolism, excretion or toxicity (ADMET) characteristics at the initial stages of the drug discovery pipeline, 3) a large repertoire of scorable phenotypes, 4) the multi-cellular and multi-organ system complexity existing in a whole organism improves the chances of identifying drugs that will ultimately be more efficacious in more complex multicellular organisms such as humans, and 5) the availability of time-proven genetic tools and genomic resources (e.g., RNAi-feeding library) simplifies drug target identification. Despite those advantages, disease modeling for clinical variants has been elusive and therefore a need exists for disease modeling that directly corresponds to patient genetic variants and screening library of compounds that target those genetic variants for further evaluation.

### SUMMARY OF THE DISCLOSURE

This disclosure provides, in some embodiments, methods for identifying potential therapeutic agents as having binding capacity to a mutated target gene product, validation of that binding capacity in “humanized” non-human cell and/or animal; and, either before, after, or simultaneously with testing for activity in induced pluripotent stem cell (iPSC)-derived cells (iPSCs). In some embodiments, compounds identified by such steps can then be re-validated) in silco, followed by further validation in the aforementioned cells and/or animals, and/or using the aforementioned iPSCs. This iterative process can be repeated, in part or in whole, until a compound suitable for testing in a patient (e.g., a human patient) is found. An illustrative embodiment is illustrated in FIG. 1. Also provided by this disclosure are reagents including but not limited to cells and/or animals that can be used in such methods. Other embodiments of such methods are also contemplated herein as would be understood by those of ordinary skill in the art.

In some embodiments, this disclosure provides non-human transgenic organisms for assessing the interaction of a human therapeutic agent and a therapeutic target, the non-human transgenic host organism comprising a human heterologous gene encoding a therapeutic target sequence operably linked to a heterologous promoter selected for expression in the host organism cells, wherein: the human heterologous gene is inserted into a non-native locus of the host organism’s genome, and expression of the human heterologous gene is expressed in non-orthologous time and/or non-orthologous tissue.

In some embodiments, this disclosure provides methods for generating and/or assessing a non-human transgenic organism for assessing the interaction between a human therapeutic agent and a therapeutic target, wherein the transgenic organism has an increased sensitivity to the human therapeutic agent, the method comprising: selecting a target sequence comprising therapeutic target protein coding sequence and/or a long noncoding sequence; selecting a tissue-specific and/or time-specific regulatory sequence as a combination of a promoter sequence and a downstream untranslated region; combining the sequences by fusing the regulatory sequence to the target sequence; creating a non-human transgenic organism by inserting the combined sequence into a non-native locus of the genome of the non-human transgenic organism; and, optionally contacting the transgenic organism to the therapeutic agent and observing an elevated phenotypic response due to the activity of the transgene.

In some embodiments, this disclosure provides methods for assessing the interaction between a human therapeutic agent and a therapeutic target over-expressed in a non-human transgenic organism for increased sensitivity of the host organism to the human therapeutic agent, the method comprising: providing a non-human transgenic organism of this disclosure comprising at least one of a human heterologous sequence expressing a therapeutic target providing a modified phenotype to the organism that is distinguished from a non-genetically modified host organism phenotype in at least one statistically significant measurable difference; contacting the genetically modified host organism of step a) with one or more human therapeutic agent(s) during an incubation period; performing one or more phenotype assay(s), during or after the incubation period, to assess interaction of the human therapeutic agent and overexpressed therapeutic target; and, recording a change in the modified phenotype following the phenotype assay, whereby, human therapeutic agents are assessed and selected based on their change in the modified phenotype.

Other embodiments are also contemplated as will be understood from this disclosure by those of ordinary skill in the art.

### BRIEF DESCRIPTION OF THE DRAWINGS

FIG. 1 illustrates an exemplary Hit-to-Lead Optimization Scheme.

FIGS. 2A, 2B and 2C illustrate the effects of the expression of a heterologous gene, SLC6A4, on the pumping frequency in a humanized transgenic nematode, a measurement of electrophysiology of a nematode pharynx pumping. The phenotypic behavior assays (ScreenChip and Food Race) graph compares the humanized transgenic nematode to wildtype (e.g. nontransgenic wild-type animal). FIG. 2A illustrates a comparison between the transgenic nematode to wildtype nematode in the presence of a food stimulus. FIG. 2B illustrates a comparison between the transgenic nematodes to wildtype nematode in the presence of a food stimulus and serotonin. FIG. 2C illustrates a comparison between the transgenic nematode to wildtype nematode in the presence of a food stimulus and compound A, compound B, compound C.

FIG. 3A shows photographs of C. elegans lin-42::GFP following exposure to dafachronic acid at various concentrations.

FIG. 3B shows a phenotypic response graph, % adult survival, of C. elegans lin-42::GFP in the presence of varying concentrations of a drug, dafadine.

FIG. 3C shows a graph of an antagonist activity reporter, mtl-1::GFP fluorescent reporter, in the presence of varying concentrations of a drug, dafachronic acid.

FIG. 3D shows a graph of an antagonist activity reporter, mtl-1::GFP fluorescent reporter, in the presence of varying concentrations of a drug, dafachronic acid.

FIG. 3E shows a phenotypic response graph, % adult survival, of C. elegans in the presence of varying concentrations of a drug, dafachronic acid.

FIG. 4 STXBP1 mutant after MD simulation is shown in gray surface, with residues 255 and 406 in red; the cavity between them is shown with a small molecule docked.

FIG. 5A. Tissues specificity with split-fluor technique. The Phsp-16.2::sfCherry stress reporter has desirable neuronal expression with an undesirable gut and pharynx expression. Uses of split sfCherry creates neuronal only expression.

FIG. 5B. Variant data on hTARDBP humanized C. elegans line for paraquat induced degeneration of amphid neurons as assess by dye filling assay (10 mM PQ, 22 hr exposure, 0.002 µg/mL lipophilic fluorescent dye DiD).

FIG. 6A. Genomic map for the human ACE2 insertion construct.

FIG. 6B. PCR data confirming the presence of the desired edit in candidate C. elegans strains.

FIG. 6C. Fluorescent image confirming the presence of the hACE2::mCherry fusion in the intestinal tissues of the C. elegans organism.

The drawings illustrate generally, by way of example, but not by way of limitation, various embodiments disclosed herein.

### DETAILED DESCRIPTION

### Introduction

Embodiments of the present invention relate generally to systems for identifying therapeutic agents that may be used to treat diseases with a clinical variant etiology. In other words, a pathogenic or modified phenotype induced by a genetic mutation in an expressed gene. Clinical variants are those sequences identified from a patient (e.g., a human), typically presenting with a pathogenic phenotype, of a gene (also referred to herein as a “disease gene”). Clinical variants are typically classified as pathogenic, likely pathogenic, benign, likely benign, variant of uncertain significance (VUS), or unassessed (or of unknown significance), wherein the majority have either uncertain or unknown pathogenicity/benign classification. Clinical variants that are classified as pathogenic, or likely pathogenic, either by others or as disclosed herein, are the subject of the present disclosure.

Disclosed herein are transgenic model organisms (e.g., nematodes or zebrafish) comprising and expressing a pathogenic (or likely pathogenic) human clinical variant gene. These clinical variant genes are also referred to as “mutated target gene” and are mutated in comparison to the wild type version of the gene. The expressed mutated target gene induces a modified phenotype in the model organism that is different from the wild type gene phenotype. That modified phenotype may be at the molecular level (e.g. gene expression, such as the transciptome) or it may be physiological (e.g. movement-based phenotypes). In some embodiments, the modified phenotype induced by the mutated target gene is a “phenocopy” of a human disease, although this is not always the case. In other words, the modified phenotype measured in the transgenic model organism is unrelated to the human disease associated with the clinical variant. Any difference between a transgenic organism expressing a wild type version of the human clinical variant and the transgenic organism expressing a pathogenic or likely pathogenic clinical variant is sufficient for the present methods, wherein that difference is measured as the modified phenotype. In exemplary embodiments, the difference in modified phenotype is a statistically significant measurable difference (e.g., about any of 5, 10, 15, 20, 25, 30, 35, 40, 45, 50, 55, 60, 65, 70, 75, 80, 85, 90, 95, 100, 150, 200, 250, 300, 350, 400, 450, 500, or more percent, and statistically significant) between animals expressing wild type and clinical variant genes.

Disclosed herein are methods using the transgenic model organisms comprising and expressing a pathogenic (or likely pathogenic) human clinical variant gene to screen or identify therapeutic agents for treatment of a disease. Therapeutic agents are identified when the clinical variant phenotype reverts to the wild type phenotype after those respective transgenic model organisms are contacted with selected therapeutic agents. Frequently the mutated target gene (pathogenic (or likely pathogenic) human clinical variant gene) is the drug target and the selected therapeutic agents are either agonists or antagonists for that gene. The present methods may be iterative and/or incorporate modeling, such as in silico methods, to identify therapeutic agents that may be useful in treatment of disease induced by a clinical variant.

In certain other embodiments, the clinical variant is not the drug target. In some embodiments, the drug target is upstream or downstream of an interaction/signaling pathway that is altered by the clinically-observed variation. In that instance, the transgenic model organism comprises and expresses two or more human genes. In some embodiments, such as using zebrafish, the endogenous locus is used. The first is a clinical variant disclosed above, and the cause of the human disease, and the second is a human gene that is the drug target. The same methods disclosed herein are used to identify therapeutic agents, except the therapeutic agent is an agonist or antagonists of the drug target gene, wherein the up or down regulation of that gene causes the modified phenotype induced by the clinical variant to change (e.g., revert to wild type). In some embodiments, the human clinical variants are modeled in the native gene of the organism.

### Definitions

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

“Clustered Regularly Interspaced Short Palindromic Repeats” and “CRISPRs”, as used interchangeably herein refers to loci containing multiple short direct repeats that are found in the genomes of approximately 40% of sequenced bacteria and 90% of sequenced archaea.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

“cDNA” as used herein means the deoxyribonucleic acid sequence that is derived as a copy of a mature messenger RNA sequence and represents the entire coding sequence needed for creation of a fully functional protein sequence.

As used herein, the terms “disrupt,” “disrupted,” and/or “disrupting” in reference to a gene mean that the gene is degraded sufficiently such that it is no longer functional. In embodiments, the native ortholog gene is replaced with the chimeric heterologous gene effectively disrupting the native host gene.

As used herein, the term “gene editing” refers to a type of genetic engineering in which DNA is inserted, replaced, or removed from a genome using gene editing tools. Examples of gene editing tools include, without limitation, zinc finger nucleases, TALEN and CRISPR.

“Genetic disease” as used herein refers to a disease, partially or completely, directly or indirectly, caused by one or more abnormalities in the genome, especially a condition that is present from birth. The abnormality may be a mutation, an insertion or a deletion. The abnormality may affect the coding sequence of the gene or its regulatory sequence. The genetic disease may be, but is not limited to epilepsy, DMD, hemophilia, cystic fibrosis, Huntington’s chorea, familial hypercholesterolemia (LDL receptor defect), hepatoblastoma, Wilson’s disease, congenital hepatic porphyria, inherited disorders of hepatic metabolism, Lesch Nyhan syndrome, sickle cell anemia, thalassaemias, xeroderma pigmentosum, Fanconi’s anemia, retinitis pigmentosa, ataxia telangiectasia, Bloom’s syndrome, retinoblastoma, and Tay-Sachs disease. “Clinical variants” are used herein, are those genes that lead to a genetic disease wherein expression of the gene results in one or more amino acid changes as compared to wild type allele that does not lead to disease.

A “heterologous gene” as used herein refers to a nucleotide sequence not naturally associated with a host animal into which it is introduced, including for example, exon coding sequences from a human gene introduced, as a chimeric heterologous gene, into a host nematode.

The term “homolog” refers to any gene that is related to a reference gene by descent from a common ancestral DNA sequence. The term “ortholog” refers to homologs in different species that evolved from a common ancestral gene by speciation. Typically, orthologs retain the same or similar function despite differences in their primary structure (mutations).

As used herein, the term “homology driven recombination” or “homology direct repair” or “HDR” is used to refer to a homologous recombination event that is initiated by the presence of double strand breaks (DSBs) in DNA (Liang et al. 1998); and the specificity of HDR can be controlled when combined with any genome editing technique known to create highly efficient and targeted double strand breaks and allows for precise editing of the genome of the targeted cell; e.g. the CRISPR/Cas9 system (Findlay et al. 2014; Mali et al. February 2014; and Ran et al. 2013).

As used herein, the term “enhanced homology driven insertion or knock-in” is described as the insertion of a DNA construct, more specifically a large DNA fragment or construct flanked with homology arms or segments of DNA homologous to the double strand breaks, utilizing homology driven recombination combined with any genome editing technique known to create highly efficient and targeted double strand breaks and allows for precise editing of the genome of the targeted cell; e.g. the CRISPR/Cas9 system. (Mali et al. February 2013).

As used herein, the terms “increase,” “increased,” “increasing,” “improved,” (and grammatical variations thereof), describe, for example, an increase of at least about 5%, 10%, 15%, 20%, 25%, 35%, 50%, 75%, 80%, 85%, 90%, 95%, 97%, 98%, 99%, 100%, 200%, 300%, 400%, 500% or more) as compared to a control. In embodiments, the increase in the context of a heterogenous gene or clinical variant thereof, is measured and/or determined via phenotypic assay to assess function of the expressed gene.

As used herein, the term “genomic locus” or “locus” (plural loci) is the specific location of a gene or DNA sequence on a chromosome and, can include both intron or exon sequences of a particular gene. A “gene” refers to stretches of DNA or RNA that encode a polypeptide or an RNA chain that has functional role to play in an organism and hence is the molecular unit of heredity in living organisms. For the purpose of this invention it may be considered that genes include regions which regulate the production of the gene product, whether or not such regulatory sequences are adjacent to coding and/or transcribed sequences. Accordingly, a gene includes, but is not necessarily limited to, introns, exons, promoter sequences, terminators, translational regulatory sequences such as ribosome binding sites and internal ribosome entry sites, enhancers, silencers, insulators, boundary elements, 5′ or 3′ regulatory sequences, replication origins, matrix attachment sites and locus control regions. As used herein “native locus” refers to the specific location of a host gene (e.g., ortholog to the heterologous gene) in a host animal.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. As used herein, “clinical variant” is a disease gene that comprises one or more amino acid changes as compared to wild type and is thus a mutant gene.

A “normal” or “wild type” nucleic acid, nucleotide sequence, polypeptide or amino acid sequence refers to a naturally occurring or endogenous nucleic acid, nucleotide sequence, polypeptide or amino acid sequence that has not undergone a change. As used herein, the wild type sequence may be a disease gene, but does not comprise a mutation leading to a pathogenic phenotype. It is understood there is a distinction between a wild type disease gene (e.g. those without a mutation leading to a pathogenic phenotype and may be an allele reflective of a “normal” heterogenous population) and clinical variants that comprise one or more mutations of those disease genes and that may have a pathogenic phenotype. In embodiments, the normal gene or wild type gene may be the most prevalent allele of the gene in a heterogenous population.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

“Partially-functional” as used herein describes a protein that is encoded by a mutant gene and has less biological activity than a functional protein but more than a non-functional protein. In embodiments, function is determined via one or more phenotypic assays wherein a phenotypic profile for the mutant (disease) gene may be generated.

As used herein, the term “percent sequence identity” or “percent identity” refers to the percentage of identical nucleotides in a linear polynucleotide of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence

As used herein, the term “percent sequence similarity” or “percent similarity” refers to the percentage of near-identical nucleotides in a linear polynucleotide of a reference (“query”) polynucleotide molecule (or its complementary strand) as compared to a test (“subject”) polynucleotide molecule (or its complementary strand) when the two sequences are optimally aligned. In some embodiments, “percent similarity” can refer to the percentage of near-identical amino acids in an amino acid sequence. Near-identical amino acids are residues with similar biophysical properties (e.g., the hydrophobic leucine and isoleucine, or the negatively-charged aspartic acid and glutamic acid).

As used herein, the term “polynucleotide” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA as DNA construct, mRNA, and anti-sense RNA, any of which can be single stranded or double stranded. The terms “polynucleotide,” “nucleotide sequence” “nucleic acid,” “nucleic acid molecule,” and “oligonucleotide” are also used interchangeably herein to refer to a heteropolymer of nucleotides. Except as otherwise indicated, nucleic acid molecules and/or polynucleotides provided herein are presented herein in the 5′ to 3′ direction, from left to right and are represented using the standard code for representing the nucleotide characters as set forth in the U.S. sequence rules, 37 CFR §§1.821 — 1.825 and the World Intellectual Property Organization (WIPO) Standard ST.25.

“Promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents.

As used herein, the terms “reduce,” “reduced,” “reducing,” “reduction,” “diminish,” “suppress,” and “decrease” (and grammatical variations thereof), describe, for example, a decrease of at least about 5%, 10%, 15%, 20%, 25%, 35%, 50%, 75%, 80%, 85%, 90%, 95%, 97%), 98%), 99%), or 100% as compared to a control. In embodiments, the reduction in the context of a heterogenous gene or clinical variant thereof, is measured and/or determined via phenotypic assay to assess function of the expressed gene.

The term “safe harbor” locus as used herein refers to a site in the genome where transgenic DNA (e.g., a construct) can be added whose expression is insulated from neighboring transcriptional elements such that the transgene expression is fully depend on only the introduced transgene regulatory elements. In certain embodiments, the present invention involves incorporation and expression of transgenic DNA includes transgenes within a safe harbor locus.

As used herein “sequence identity” refers to the extent to which two optimally aligned polynucleotide or peptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the phrase “substantially identical,” or “substantial identity” and grammatical variations thereof in the context of two nucleic acid molecules, nucleotide sequences or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100%> nucleotide or amino acid residue identity, when compared and aligned for maximum correspondence, as measured using one of the following sequence comparison algorithms or by visual inspection. In particular embodiments, substantial identity can refer to two or more sequences or subsequences that have at least about 70%, at least about 75%, at least about 80%, at least about 85%, at least about 90%, at least about 95, 96, 96, 97, 98, or 99% identity.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always > 0) and N (penalty score for mismatching residues; always < 0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=-4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat’l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001. Thus, in some embodiments of the invention, the smallest sum probability in a comparison of the test nucleotide sequence to the reference nucleotide sequence is less than about 0.001.

“Subject” and “patient” as used herein interchangeably refers to any vertebrate, including, but is not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus or rhesus monkey, chimpanzee, etc.) and a human). In some embodiments, the subject may be a human or a non-human. The subject or patient may be undergoing other forms of treatment. In embodiments, the patient is a human wherein a clinical variant is a sequence of a disease gene from the patient.

“Target gene” as used herein refers to any nucleotide sequence encoding a known or putative gene product. As used herein the target gene may be the chimeric heterologous gene, either in normal or wild type form, or as a clinical variant, or the host animal ortholog of the heterologous gene. The target gene may be a mutated gene involved in a genetic disease, also referred to herein as a clinical variant.

“Target nucleotide sequence” as used herein refers to the region of the target gene to which the Type I CRISPR/Cas system is designed to bind.

The terms “transformation,” “transfection,” and “transduction” as used interchangeably herein refer to the introduction of a heterologous nucleic acid into a cell. Such introduction into a cell may be stable or transient. Thus, in some embodiments, a host cell or host organism is stably transformed with a polynucleotide of the invention. In other embodiments, a host cell or host organism is transiently transformed with a polynucleotide of the invention. “Transient transformation” in the context of a polynucleotide means that a polynucleotide is introduced into the cell and does not integrate into the genome of the cell. By “stably introducing” or “stably introduced” in the context of a polynucleotide introduced into a cell is intended that the introduced polynucleotide is stably incorporated into the genome of the cell, and thus the cell is stably transformed with the polynucleotide. “Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein also includes the nuclear, the plasmid and the plastid genome, and therefore includes integration of the nucleic acid construct into, for example, the chloroplast or mitochondrial genome. Stable transformation as used herein can also refer to a transgene that is maintained extra-chromosomally, for example, as a mini-chromosome or a plasmid. In certain embodiments, the nucleotide sequences, constructs, expression cassettes can be expressed transiently and/or they can be stably incorporated into the genome of the host organism, such as in a native, non-native locus or safe harbor location.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism’s genetic code. The introduction of a transgene has the potential to change the phenotype of an organism.

The term “3′untranslated region” or “3′UTR” refers to a nucleotide sequence downstream (i.e., 3′) of a coding sequence. It generally extends from the first nucleotide after the stop codon of a coding sequence to just before the poly(A) tail of the corresponding transcribed mRNA. The 3′ UTR may contain sequences that regulate translation efficiency, mRNA stability, mRNA targeting and/or polyadenylation. In embodiments, the 3′ UTR may be native, or non-native in the context of the chimeric heterologous gene sequence.

“Variant” with respect to a peptide or polypeptide that differs in one or more amino acid sequence by the insertion, deletion, or conservative substitution of amino acids as compared to a normal or wild type sequence. The variant may further exhibit a phenotype that is quantitatively distinguished from a phenotype of the normal or wild type expressed gene. In embodiments, clinical variant refers to a disease gene with one or more amino acid changes as compared to the normal or wild type disease gene.

The terms “transformation,” “transfection,” and “transduction” as used interchangeably herein refer to the introduction of a heterologous nucleic acid into a cell. Such introduction into a cell may be stable or transient. Thus, in some embodiments, a host cell or host organism is stably transformed with a polynucleotide of the invention. In other embodiments, a host cell or host organism is transiently transformed with a polynucleotide of the invention. “Transient transformation” in the context of a polynucleotide means that a polynucleotide is introduced into the cell and does not integrate into the genome of the cell. By “stably introducing” or “stably introduced” in the context of a polynucleotide introduced into a cell is intended that the introduced polynucleotide is stably incorporated into the genome of the cell, and thus the cell is stably transformed with the polynucleotide. “Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein also includes the nuclear, the plasmid and the plastid genome, and therefore includes integration of the nucleic acid construct into, for example, the chloroplast or mitochondrial genome. Stable transformation as used herein can also refer to a transgene that is maintained extrachromosomally, for example, as a mini-chromosome or a plasmid. In certain embodiments, the nucleotide sequences, constructs, expression cassettes can be expressed transiently and/or they can be stably incorporated into the genome of the host organism, such as in a native, non-native locus or safe harbor location.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism’s genetic code. The introduction of a transgene has the potential to change the phenotype of an organism.

The term “3′untranslated region” or “3′UTR” refers to a nucleotide sequence downstream (i.e., 3′) of a coding sequence. It generally extends from the first nucleotide after the stop codon of a coding sequence to just before the poly(A) tail of the corresponding transcribed mRNA. The 3′ UTR may contain sequences that regulate translation efficiency, mRNA stability, mRNA targeting and/or polyadenylation. In embodiments, the 3′ UTR may be native, or non-native in the context of the chimeric heterologous gene sequence.

“Variant” with respect to a peptide or polypeptide that differs in one or more amino acid sequence by the insertion, deletion, or conservative substitution of amino acids as compared to a normal or wild type sequence. The variant may further exhibit a phenotype that is quantitatively distinguished from a phenotype of the normal or wild type expressed gene. In embodiments, clinical variant refers to a disease gene with one or more amino acid changes as compared to the normal or wild type disease gene.

### Drug Screening Systems

This disclosure relates to drug screening systems for identifying mutated target gene(s) (and/or corresponding mutated proteins; e.g., “clinical variant(s)”, “genetic variant(s)”) involved in and/or compounds useful in preventing and/or treating disease conditions in animals (including human beings) directly or indirectly caused by such mutated target gene(s), and reagents for use in such systems. In some embodiments, the reagents can include cells and/or transgenic animals expressing said mutated target gene(s), as well as methods for preparing and using the same. In some embodiments, the cell and/or transgenic animal expresses a detectable phenotypic (e.g., physical) change upon treatment with a test compound (e.g., an agonistic or antagonistic drug) in a cell or animal expressing the mutated target gene(s) (i.e., that can affect the function of a “mutated target gene” or a target thereof). In some embodiments, the detectable phenotypic change is a statistically significant measurable difference (e.g., about any of 5, 10, 15, 20, 25, 30, 35, 40, 45, 50, 55, 60, 65, 70, 75, 80, 85, 90, 95, 100, 150, 200, 250, 300, 350, 400, 450, 500, or more percent, and statistically significant as compared to the phenotype of a corresponding cell and/or animal that does not express the mutated target gene(s). In some embodiments, a detectable phenotypic change results from expression of the human target gene(s) (e.g., clinical variant(s)) at the corresponding locus of the cell and/or animal genome (e.g., the “natural” locus, an orthologous position in the genome), or from expression at a different locus (e.g., a non-“natural” locus, a non-orthologous position in the genome). In some embodiments, detectable phenotypic change occurs from insertion of a clinical variant at the endogenous gene locus. In some embodiments, functional data from mosaic F0 animals modified with a clinical variant at animal’s endogenous locus is obtained, and could be modified to include single cell analysis. In some embodiments, the phenotypic change results from the exposure of the cells and/or animals to a drug having an activity that is in some way affected by, or affects the activity of, the mutated target gene or a target thereof.

In some embodiments, expression of the mutated target gene in the transgenic animal can affect (e.g., influence the activity of) one or more “therapeutic target(s)” in the cell and/or animal and have a phenotypic effect. In some embodiments, a therapeutic target can be a gene product that interacts with a drug (a “drug target”), where the gene encoding such product can be a “drug target gene”. In some embodiments, a cell and/or animal can be engineered to express combination(s) of one or more mutated target genes along with one or more wild-type (e.g., non-mutated) drug target genes that encode one or more drug targets that serve as targets for a drug to be tested. Expression of such combinations in a cell and/or animal can be used to identify drug(s) that affect the relationship between the drug and the drug target in the presence of the product of the mutated drug product gene. For instance, a cell and/or animal can be engineered to express one or more STXBP1 human protein clinical variant(s) (mutant target gene(s)) along with wild-type CNR1 and/or CNR2 “drug target genes” where the drug is, in some embodiments, a cannabinoid (e.g., cannabidiol). In such exemplary embodiments, the effect of the one or more STXBP1 human protein variant(s) on the activity/function of the CNR1 and/or CNR2 proteins in the cell or animal can be determined by measuring the phenotypic effects of cannabidiol thereupon. In some embodiments, these reagents and methods can be used to study the effects of other drugs on the interaction of the members of such combinations can also be studied (e.g., a second drug may be introduced to measure any effect on the STXBP1 human protein clinical variant(s)/CNR1/CNR2/cannabidiol relationship). Such phenotypic effects can be compared to the phenotypic effects observed in a cell and/or animal expressing CNR1 and/or CNR2 but not the one or more STXBP1 human protein clinical variants, where in some embodiments the measurable difference in the phenotypic effect is a statistically significant measurable difference (e.g., about any of 5, 10, 15, 20, 25, 30, 35, 40, 45, 50, 55, 60, 65, 70, 75, 80, 85, 90, 95, 100, 150, 200, 250, 300, 350, 400, 450, 500, or more percent, and statistically significant). Such systems and methods can be used to identify treatments for human beings expressing such mutated target genes clinical variants that use such drugs (e.g., cannabidiol for epilepsy).

In some embodiments, this disclosure provides cells and/or animals can be engineered to express a therapeutic target gene and a mutated target gene (e.g., “double humanized patient allele models”). For instance, human gamma-aminobutyric acid transaminase (hGABAT (an exemplary “therapeutic target gene”)) is an important target for anti-epilepsy drugs such as valproic acid and vigabatrin, even when an hGABAT clinical variant is not present in the treated patient. Literature evidence can also be found of the effectiveness of vigabatrin for patients expressing one or more mutated target genes (e.g., hSTXBP1 variants (Romaniello, et al. J. Child Neurology, 29(2): 249-253 (2014)), hKCNQ2 variants (Lee, et al. Pediatr Neurol. 40(5): 387-91 (2009)), and hCDKL5 variants (Melikishvili et al. Epilepsy Behav. 94:308-311 (2019), among others). Such double humanized patient allele models can then be treated with drugs targeting one or both of the alleles (e.g., drugs targeting hGABAT and/or hSTXBP1 variants) followed by measuring of the effects of such treatment on phenotype.

In some embodiments, the phenotypic change in the cell or animal corresponds to the activity of the transgene (or therapeutic target) in its natural host (e.g., as a human target gene or human therapeutic target would function in a human being), and in some embodiments it does not. In other words, in some embodiments, the clinical variant phenotype may not be a “phenocopy” of the human disease. The methods described herein only require a phenotypic change that can be detected in the transgenic animal upon expression of the clinical variant. In some embodiments, the phenotypic change provides a statistically significant measurable difference (e.g., about any of 5, 10, 15, 20, 25, 30, 35, 40, 45, 50, 55, 60, 65, 70, 75, 80, 85, 90, 95, 100, 150, 200, 250, 300, 350, 400, 450, 500, or more percent, and statistically significant) in phenotype from control (e.g., an animal not expressing the target gene). In some embodiments, transcription of a target gene can be directed to a particular cell type and/or tissue (e.g., in some embodiments a C. elegans myo-2 promoter can be operably linked to the humanized gene to induce expression of the protein encoded in the pharynx muscle cells). For instance, in some embodiments, a non-human animal cells (e.g., C. elegans) can be transduced with a target gene expressed at high levels in the pharynx that causes altered pharyngeal pump frequency, pump duration and pump interval detectable and observable in electrophysiological data measured by an appropriate assay system. In some embodiments, expression of the target gene can be unlimited, or generalized, for different types of cells and/or throughout an animal (e.g., a snb-1 pan-neuronal promoter sequence can be operably linked to (e.g., inserted upstream of) the humanized gene start codon resulting in excessive and broad overexpression; snb-1 referring to the C. elegans synaptobrevin-1 protein encoding gene).

In some embodiments, this disclosure provides methods for identifying one or more therapeutic agents to prevent and/or treat a condition associated with the altered function of a gene (e.g., mutated target gene, human clinical variant), the method comprising: a) identifying a first test compound from a collection of compounds by in silico molecular dynamic simulation of the interaction of members of said collection of compounds with a test protein encoded by a mutated target gene, wherein a compound that interacts with the test protein is a first test compound (i.e., one or more potential therapeutic agents); b) b1) incubating a cell or animal with one or more first test compounds, wherein: the cell or animal expresses the at least one mutated target gene, optionally optimized for expression in the cell or animal; the mutated target gene induces a modified phenotype in the cell or animal that differs from a normal phenotype induced by expression of a non-mutated version of the mutated target gene in the cell or animal; and, the modified phenotype results in at least a statistically significant measurable difference (e.g., about any of 5, 10, 15, 20, 25, 30, 35, 40, 45, 50, 55, 60, 65, 70, 75, 80, 85, 90, 95, 100, 150, 200, 250, 300, 350, 400, 450, 500, or more percent, and statistically significant) from the normal phenotype; and, b2) identifying second test compound(s) that transform the modified phenotype of the cell or animal into a normal phenotype; and/or, c) 1) incubating induced pluripotent stem cells (iPSCs) derived from a human patient expressing said mutated target gene therein with one or more first test compounds, and/or one or more second test compounds, wherein: c1) the mutated target gene induces a modified phenotype in the iPSCs that differs from the normal phenotype induced by expression of a non-mutated version of the mutated target gene in iPSCs derived from a human patient that does not express the mutated target gene and, the modified phenotype results in at least a statistically significant measurable difference (e.g., about any of 5, 10, 15, 20, 25, 30, 35, 40, 45, 50, 55, 60, 65, 70, 75, 80, 85, 90, 95, 100, 150, 200, 250, 300, 350, 400, 450, 500, or more percent, and statistically significant) from the normal phenotype; and, c2) identifying third test compound(s) that transform the modified phenotype of the iPSCs into a normal phenotype as second test compound(s); and, optionally, d) repeating steps a) and b); a) and c); or, a), b) and c); to identify additional test compounds. Other embodiments of such methods are also contemplated herein as would be understood by those of ordinary skill in the art.

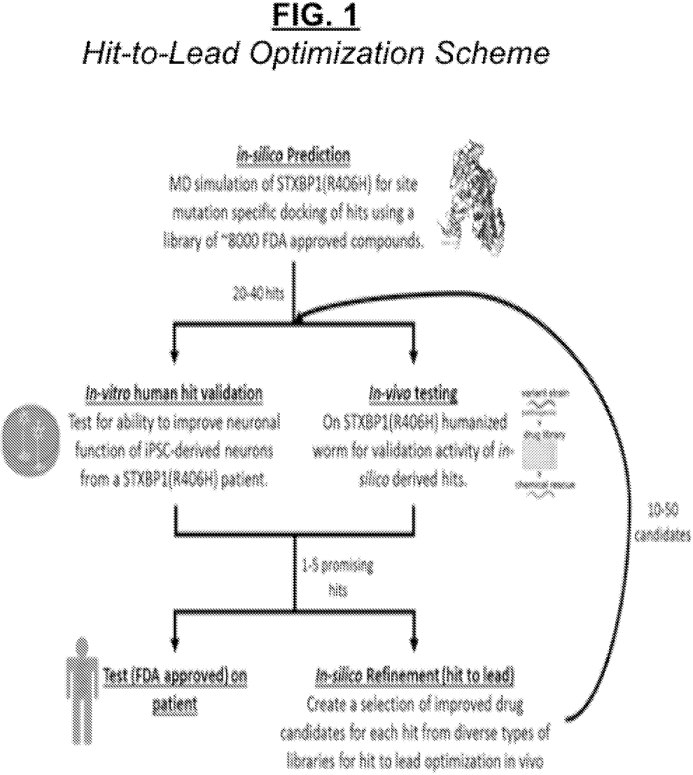

An illustrative embodiment of such methods is illustrated in FIG. 1. As illustrated therein, the first step can include molecular dynamic (MD) simulation of site-mutation specific docking of compounds of a library (e.g., comprising thousands of FDA-approved drugs) at one or more particular sites of a human protein of interest (e.g., STXBP1 comprising the R406H mutation). The compounds identified as having binding capacity in the first step (e.g., 20-40 “hits” as illustrated in FIG. 1), can then be tested (e.g., validated) for the ability to change (e.g., “improve”) the function (e.g., neuronal function) of induced pluripotent stem cell (iPSC)-derived cells (e.g., iPSC-derived neurons) isolated from cells of a patient (e.g., skin cells). Such compounds can also be tested (e.g., validated) in a “humanized” non-human cell (e.g., C. elegans or zebrafish cells) or animal (e.g., a transgenic animal such as C. elegans or zebrafish); either before, after, or simultaneously with the aforementioned iPSC testing. Compounds validated in this step (e.g., a subset of 1-5 compounds) can then be re-tested (e.g., re-validated) in silco, followed by further validation using the aforementioned iPSC testing and/or using the aforementioned humanized non-human cells or animals. This iterative process can be repeated, in part or in whole, until a compound suitable for testing in a patient (e.g., a human patient). Other embodiments of such methods are also contemplated herein as would be understood by those of ordinary skill in the art.

The information generated using these reagents and methods can be used in multiple applications, such as for pharmaceutical development and/or to identify patients that may or may not be candidates for treatment with a particular drug. For instance, in some embodiments, a CNR transgenic animal (e.g., nematode) can comprise a CNR gene introduced into the animal in combination with a clinical variant to study a particular disease (e.g., for epilepsy).

### Transgenic Non-Human Animals

In some embodiments, this disclosure provides cells (e.g., cell lines) and/or non-human animal(s) (e.g., transgenic animals) that expresses the at least one mutated target gene, optionally optimized for expression in the cell or animal; wherein the mutated target gene induces a modified phenotype in the cell and/or animal that differs from a normal phenotype induced by expression of a non-mutated version of the mutated target gene in the cell(s) and/or animal(s); and wherein the modified phenotype results in at least a statistically significant measurable difference (e.g., about any of 5, 10, 15, 20, 25, 30, 35, 40, 45, 50, 55, 60, 65, 70, 75, 80, 85, 90, 95, 100, 150, 200, 250, 300, 350, 400, 450, 500, or more percent, and statistically significant). As used herein, transgenic organisms include both a mutation and/or heterologous gene inserted into the genome and expressed and transient expression of a transgene from an expression vector. In some embodiments, as in the methods disclosed herein, one or more potential therapeutic agents can be identified that transform the modified phenotype of the cell or animal into a normal phenotype. In embodiments, the animal is a vertebrate selected from an avian, a fish, a reptile, a mammal, or an amphibian. In other embodiments, the animal is an invertebrate selected from a Porifera, a Cnidaria, a Platyhelmintes, a Nematoda, an Annelida, a Mollusca, an Arthropoda, or an Echinodermata. In certain embodiments, the animal is a nematode (e.g., C. elegans), a fruit fly, a zebrafish or a frog (e.g., xenopus). In further embodiments, the animal is a metazoan. In other embodiments, the animal is a primate, mammal, rodent or fly. In embodiments, the animal is a parasite species. In other embodiments, the animal is a Chordata, Actinopterygii or Nematoda. In specific embodiments, the animal is Danio rerio zebrafish or C. elegans nematode.

In some such embodiments, one or more therapeutic target gene(s) (e.g., a clinical variant) can be first optimized for expression in the host animal (e.g. zebrafish, nematode), and then incorporated into the genome of that host animal to produce a transgenic animal. In some embodiments, tissue-specific promoters such as odr-10, snb-1; conditional promoters such as hsp-16; native promoters such as npr-19; ubiquitous promoter such as eft-3; 3′-untranslated regions such as tbb-2, npr-19, eft-3, and/or unc-54; and/or selection markers such as unc-119, hygromycin resistance, fluorescent marker(s), rol-6, and/or cha-1 rescue; may be used to produce such cells and/or animals. Other embodiments of such methods are also contemplated herein as would be understood by those of ordinary skill in the art.

Such transgenic animals can be prepared using nucleic acid constructs as has been described previously (e.g., creating transgenic C. elegans lines using the MosSCI method of Frøkjær-Jensen, et al. (Nat Genet. 40(11): 1375-1383 (2008)). Briefly, to produce transgenic C. elegans, an injection mix can be created using multiple components such as the target plasmid pNU2006 (e.g., at 15 ng/ul), an eft-3p::Mosase pNU272 plasmid (e.g., at 10 ng/ul) to provide the transposase activity, plasmids encoding fluorescent proteins for visual indication (e.g., red fluorescent protein plasmids pGH8 (at 10 ng/ul), pCFJ104 (at 10 ng/ul), and pCFJ90 (at 1.25 ng/ul) for mCherry expression controlled by the rab-3, myo-3, and myo-2 promoters respectively; and/or other fluorescent plasmid markers), plasmid(s) providing selection against extrachromosomal arrays (e.g., the pMA122 plasmid at, e.g., 10 ng/ul, which has a heat shock inducible expression of the toxic protein peel-1). The components are typically mixed, typically also including water to a particular volume (e.g., 20 ul). C. elegans are typically prepared for injection by growth on HB101 bacteria. The injected strain is COP93 which are derived from EG6699 — ttTi5605 II; unc-119(ed3) III; oxEx1578 [eft-3p::GFP + Cbr-unc-119] — by selecting against oxEx1578. The strain generated has a Mos1 insertion at ttTi5605 on Chromosome II and the unc-119(ed3) mutation in the background. Upon activation by the Mosase transposase, the Mos1 “hops” out of the genome creating a double-stranded break. The repair of this break by homologous recombination can be co-opted to also introduce DNA sequences that are between the two homology arms. To achieve this, young adult animals are selected for injections. Animals are injected into the gonad using a micropipette needle containing the injection mix. After injection animals are recovered, incubated and allowed to reproduce on Nematode Growth Media plates. When the progeny animals have cleared the plate of food (approximately 7 days), these are assayed for rescue of the unc-119 movement phenotype. Heat shock is typically then performed at 34° C. for 4 hrs. Two days later, animals rescued for the unc-119 movement phenotype without the red fluorescent array markers are then selected, and PCR is used to confirm integration at the locus and the absence of the Mos1 sequence. Other methods are also available as is understood by those of ordinary skill in the art.

Provided herein is a transgenic zebrafish system for assessing function of a heterologous gene, wherein the heterologous gene is wild type, or a variant thereof. In embodiments, the system comprises a host zebrafish comprising a chimeric heterologous gene comprising heterologous exon coding sequences interspersed with artificial host zebrafish intron sequences optimized for expression in the host zebrafish wherein the chimeric heterologous gene replaced an entire host zebrafish gene ortholog at a native locus and expression of the heterologous gene at least partially restores function of the replaced zebrafish ortholog providing a validated transgenic zebrafish, and wherein the heterologous gene is a eukaryotic gene. In embodiments, the system comprises a test transgenic zebrafish comprising a chimeric variant heterologous gene, comprising human exon coding sequences interspersed with artificial host zebrafish intron sequences optimized for expression in the host zebrafish, wherein the exon coding sequences comprise one or more mutations resulting in an amino acid change as compared to a wildtype reference sequence, wherein the chimeric variant heterologous gene replaced a host zebrafish gene ortholog at a native locus. Also provided herein is a method of preparing a transgenic zebrafish comprising a chimeric heterologous gene. In embodiments, the methods comprise optimizing a heterologous gene coding sequence for expression in a host zebrafish comprising selecting host optimized codons, adding artificial host zebrafish intron sequences between exon coding sequences of the heterologous gene, and removing aberrant splice donor and/or acceptor sites to provide a chimeric heterologous gene sequence and inserting the chimeric heterologous gene sequence via homologous recombination into a native locus of the host zebrafish wherein the chimeric heterologous gene replaces an entire zebrafish ortholog gene at the native locus, and wherein expression of the heterologous gene at least partially restores function of the replaced zebrafish ortholog, wherein the heterologous gene is a eukaryotic gene.

Alternative methods for generation of transgenic cell and/or animal lines are also available including extrachromosomal array and CRISPR/Cas9, as in known in the art. CRISPR techniques can be deployed to directly mutate genes and the like within the genome of a cell and/or animal loci. (Kim H et al. Genetics. Aug;197(4):1069-80 (2014); Farboud, et al. Genetics. 199(4):959-71 (2015); and, Paix et al. Genetics. 201(1):47-54 (2015)). In some embodiments, the clinical variant can be incorporated into the genome of a cell and/or animal by CRISPR as an amino-acid-swap which substitutes the native amino acid with the amino acid change seen in the patient. Briefly, in some embodiments, injections are performed with a dpy-10 sgRNA and a dpy-10 oligonucleotide repair template in the injection mix, and homology-mediated mutagenesis of a dpy-10 locus can be used to detect which injections have a high transformation potential. Typically an injection mix includes a set of sgRNAs targeting a clinical variant editing locus, another repair template instructing for content of clinical variant edit, and Cas9 protein. Typically, ~20 animal gonads are injected with approximately 10-50 nl of injection mix, and three to five days later populations with high frequency of Rol phenotype are identified and isolated for population expansion. After egg lay, the adults are harvested, and PCR is specifically designed to distinguish between homozygous mutant, homozygous wild-type and heterozygous animals is carried out. Animals from populations PCR positive for the mutation are isolated for population expansion and, after egg lay, the adult is PCR tested again to detect presence of homozygosity. Mutations are confirmed by sequencing. The examples describe an embodiment using with CRISPR/Cas9 system to create animals expressing SLC6A4 variants (Gly56Ala and Lys605Asn), and the identification of SLC6A4 antagonists using those animals (e.g., the Gly56Ala and Lys605Asn lines are treated with compound and pharyngeal pumping is tested with the ScreenChip™; a significant difference from wild-type being an indicator of potential drug effectiveness).

In some embodiments, a zebrafish gene knock out of a target ortholog can be obtained from either genetic stock centers or made with gene knock-out techniques (e.g., CRISPR-based gene deletion). Next, a humanizing transgene mRNA coding for the human ortholog sequence can be obtained and used to rescue function. In another example, a morpholino RNAi is used to knock down expression of a target ortholog gene and a humanizing mRNA is introduced to rescue gene function. Once rescue of function is achieved, genetic variants are inserted into the humanizing RNA sequence and defects of rescue capacity are measured and quantified. For instance, a knockout line for the Zebrafish stxbp1a gene can be created by CRISPR/Cas9. sgRNAs targeting early in the coding sequence, exon 3, were used to create cuts in the sequence coding for amino acids 38 and 45 (sgRNA sequences: TAGTGGACCAGCTCAGCATG (SEQ ID NO: 101) and GATATCAGTCATTTTGCAGC (SEQ ID NO: 102)). Zebrafish lines with germline transmitting mutations that lead to an early stop are selected. Embryos are injected with human mRNA for STXBP1 or Zebrafish mRNA for Stxbp1a and rescue of movement and lethality is measured and compared with mCherry mRNA injected controls. Variant mutations are introduced into the plasmid with the STXBP1 mRNA expression construct. mRNA with the variants are produced and injected into the knockout zebrafish lines. Movement and lethality phenotypes are measured and compared to the wildtype human mRNA control. This is a rapid method for variant assessment using a vertebrate system.

In some embodiments, the methods disclosed herein are agonist and/or antagonist detection systems (e.g., G protein-coupled receptor (GPCR) agonists), wherein a human gene (e.g., mutated target gene) is first inserted into the genome of an animal (e.g., C. elegans, zebrafish) under the control of an orthologous native promoter such that the human gene is expressed in. In some embodiments, such as when the target gene is capable of rescuing function, the promoter can be “bashed” by random mutagenesis to create downward attenuated expression. For instance, existing ChIP-seq data is used to determine promoter binding elements region upstream of transgene start codon. CRISPR-mediated donor homology insertion with ODN templating can be used to alter codon composition in the promoter. Alternatively, nontemplated CRISPR-mediated error-prone repair can be used. When a promoter-bashed animal starts to exhibit a phenotype similar to the knockout (KO) allele, a drop in expression can be verified by reverse transcription polymerase chain reaction (rtPCR). When a transgenic animal is found to exhibit a loss-of-function (LOF) phenotype and a significant drop in transgene expression, the promoter-bashed system becomes a platform for discovery of agonists that restore behavior back to normal activity.

### Target Genes / Clinical Variants

Various classes of mutated target gene(s) (e.g., clinical variants) and/or other human genes can be incorporated into a cell and/or animal (Pathogenic, Likely Pathogenic, Uncertain Significance, Likely Benign, Benign, and the unassessed). On average, dbSNP data indicates 80% of known clinical variants are unassessed, and nearly half (40%) of the remaining assessed variants are Variants of Uncertain Significance (VUS) ((NCBI) Variation Viewer (Assessed Feb. 20, 2018)). Incorporation of known pathogenic and benign variants into cells and/or animals assists in determining how conserved are the existing assignments when incorporated into the genome of a cell and/or animal into the human cDNA expressing animal model. When most of the pathogenic and benign variants are observed to produce the expected activities in the humanized animal model (e.g., a transgenic C. elegans or zebrafish animal), the system then is valid for assessment of pathogenicity of VUS and unassigned variants.

Thus, in some embodiments, cells and/or animals (e.g., nematodes such as C. elegans, or zebrafish) can be engineered to express mutated target genes and methods of screening target agonists and antagonists used to select those that can function as therapeutic drugs in the treatment of a disease. In some embodiments using the C. elegans system, the altered transgenic nematode cell lines can be designed to provide high levels of gene expression in the pharynx, leading to abundant levels of the mutated target gene (e.g., clinical variant) or other gene at the plasma membrane of the pharynx muscle cells or other neuronal cell. Exemplary phenotypic changes induced or affected by the mutated target gene can include those leading to altered pharyngeal pump frequency, pump duration and pump interval detectable and observable as electrophysiological data. In some embodiments, a mutated target gene or other gene can be optimized for expression in the animal of interest by, e.g., incorporating artificial nematode or zebrafish introns to the respective nematode or zebrafish target sequence (e.g., also removing aberrant splice sites). The artificial introns can be designed based on small introns in highly expressed native nematode or zebrafish genes, respectively. The intron sequences maintain the coding frame such that when translated the target gene (i.e., coding sequence) does not contain stop codons and has a low hydropathy index. In one embodiment for use in C. elegans, the optimized target can be cloned into a donor homology plasmid (e.g., pNU1313), such as one including the C. elegans myo-2 promoter, and in some embodiments tbb-2 3′ untranslated region (UTR), to drive expression of the target gene in the pharynx muscle cells, as well as homology arms for insertion into the ttTi14024 Mos1 insertion site and the unc-119 rescue cassette. Such reagents and methods for using the same are known to those of ordinary skill in the art (see, e.g., Frokjaer-Jensen, et al. Nat. Methods (2012); Frøkjaer-Jensen, et al. Nat. Genet. (2008); Evans, TC Transformation and microinjection. In (The C. elegans Research Community, ed. (2006); as well as in U.S. Pat. Application 16/281,988.

Suitable mutated target genes and/or common variants thereof (e.g., clinical variants, disease-associated genes) can be identified using exome and genome sequencing databases such as genomAD, or otherwise identified as has been or can be done by those of ordinary skill in the art. A non-limiting exemplary listing of mutated target genes or other genes, i.e., those that could be mutated to cause a modified phenotype in cell(s) and/or animal(s)) suitable for use in these methods, and the corresponding C. elegans and zebrafish ortholog genes, are provided in Tables 1 (C. elegans) and 2 (zebrafish). “Tier 1” genes have the closest sequence similarity (“Similarity”) to the respective C. elegans and zebrafish orthologs, followed by Tier 2 (below 55% similarity for C. elegans genes, below 75% similarity for zebrafish genes), and then Tier 3 (below 42% similarity for C. elegans genes, below 60% similarity for zebrafish genes). The “-” symbol indicates an as-yet undetermined phenotype. The Tier 1 similarity cutoff is higher in zebrafish since model variants are typically produced directly in the zebrafish ortholog as a CRISPR-mediated amino acid substitution. This is unlike the typical procedure in C. elegans in which the C. elegans gene is entirely replaced by the human gene (or humanized, or codon-optimized gene). In preferred embodiments, the target gene(s) is/are Tier 1 genes, followed by Tier 2 genes, and then Tier 3 genes. Other genes may also be suitable as would be understood by those of ordinary skill in the art.

TABLE 1

C. elegans Orthologs

| Tier | Human | C. elegans ortholog | DIOPT Score | Best Hit | Reverse Hit | Similarity (%) | C. elgans phenotype |
|---|---|---|---|---|---|---|---|
| 1 | GFPT1 | gfat-2 | 12 | Yes | Yes | 76 | lethal |
| 1 | SPTLC2 | sptl-2 | 14 | Yes | Yes | 74 | morphology |
| 1 | PTS | ptps-1 | 15 | Yes | Yes | 71 | pumping |
| 1 | PSAT1 | F26H9.5 | 15 | Yes | Yes | 71 | gene expression |
| 1 | CAD | pyr-1 | 14 | Yes | Yes | 71 | lethal |
| 1 | TH | cat-2 | 11 | Yes | Yes | 71 | locomotion |
| 1 | PSPH | Y62E10A. 13 | 14 | Yes | Yes | 70 | development |
| 1 | SPTLC1 | sptl-1 | 13 | Yes | Yes | 70 | lethal |
| 1 | KCNT1 | slo-2 | 15 | Yes | Yes | 69 | behavior |
| 1 | SLC18A2 | cat-1 | 14 | Yes | Yes | 66 | behavior |
| 1 | ATP7A | cua-1 | 13 | Yes | Yes | 62 | lethal |
| 1 | COQ4 | coq-4 | 14 | Yes | Yes | 60 | lifespan |
| 1 | PHGDH | C31C9.2 | 13 | Yes | Yes | 58 | development |
| 1 | COQ6 | coq-6 | 13 | Yes | Yes | 58 | lethal |
| 1 | PLPBP | F09E5.8 | 11 | Yes | Yes | 57 | development |
| 1 | GLRA1 | glc-3 | 10 | Yes | Yes | 53 | behavior |
| 1 | SLC52A3 | rft-1 | 14 | Yes | Yes | 49 | development |
| 1 | TPK1 | tpk-1 | 14 | Yes | Yes | 48 | locomotion |
| 1 | NPC1 | ncr-1 | 13 | Yes | Yes | 47 | lethal |
| 1 | TSC1 | gip-1 | 1 | Yes | Yes | 33 | lethal |
| 1 | SLC13A5 | nac-3 | 13 | Yes | No | 61 | lifespan |
| 1 | SLC2A1 | fgt-1 | 11 | Yes | No | 60 | lifespan |
| 1 | MTHFS | Y106G6E. 4 | 9 | Yes | No | 56 | lifespan |
| 2 | KCNQ2 | kqt-1 | 9 | Yes | No | 54 | ephys |
| 2 | CHRNE | unc-63 | 3 | Yes | No | 52 | locomotion |
| 2 | GRIN2A | nmr-2 | 11 | Yes | No | 50 | behavior |
| 2 | SLC19A3 | folt-1 | 12 | Yes | No | 49 | lethal |
| 2 | SLC52A2 | rft-1 | 11 | Yes | No | 48 | development |
| 2 | SLC30A10 | cdf-1 | 10 | Yes | No | 45 | copper resistant |
| 2 | SCN8A | egl-19 | 3 | Yes | No | 42 | lethal |
| 2 | GOT2 | got-2.2 | 13 | Yes | Yes | 83 | – |
| 2 | ALDH7A1 | alh-9 | 12 | Yes | Yes | 80 | – |
| 2 | QDPR | qdpr-1 | 15 | Yes | Yes | 69 | – |
| 2 | PNPO | F57B9.1 | 13 | Yes | Yes | 61 | – |
| 2 | CTNS | ctns-1 | 13 | Yes | Yes | 57 | – |
| 2 | SLC39A14 | zipt-15 | 12 | Yes | Yes | 56 | – |
| 2 | TTR | R09H10.3 | 10 | Yes | Yes | 48 | – |
| 2 | SLC46A1 | Y4C6B0.5 | 10 | Yes | Yes | 46 | – |
| 2 | SLC39A4 | zipt-15 | 5 | Yes | No | 56 | – |
| 3 | SCN2A | egl-19 | 1 | Yes | No | 41 | lethal |
| 3 | CACNA1A | unc-2 | 9 | Yes | No | 40 | locomotoin |
| 3 | SCN3A | egl-19 | 1 | Yes | No | 40 | lethal |
| 3 | SCN1A | egl-19 | 1 | Yes | No | 40 | lethal |
| 3 | TSC2 | F53A10.2 | 1 | Yes | No | 36 | – |
| 3 | SLC25A19 | C42C1.19 | 7 | Yes | No | 34 | – |
| 4 | TPP1 | – | – | – | – | – | – |
| 4 | PRRT2 | – | – | – | – | – | – |
| 4 | FOLR1 | – | – | – | – | – | – |

TABLE 2

Zebrafish Orthologs

| Tier | Human | Zebrafish ortholog | DIOPT Score | Best Hit | Reverse Hit | Similarity (%) | Zebrafish phenotype |
|---|---|---|---|---|---|---|---|
| 1 | ALDH7A1 | aldh7a1 | 10 | Yes | Yes | 93 | lifespan |
| 1 | GFPT1 | gfpt1 | 10 | Yes | Yes | 92 | lifespan, small |
| 1 | SLC2A1 | slc2a1b | 14 | Yes | Yes | 91 | small, pigment |
| 1 | GLRA1 | glra1 | 11 | Yes | Yes | 89 | muscle, startle |
| 1 | SCN2A | scn1lab | 11 | Yes | Yes | 87 | lifespan, movement |
| 1 | CAD | cad | 12 | Yes | Yes | 86 | small, neurodegen |
| 1 | SLC18A2 | slc18a2 | 11 | Yes | Yes | 85 | movement |
| 1 | SCN8A | scn8aa | 14 | Yes | Yes | 82 | movement |
| 1 | COQ6 | coq6 | 15 | Yes | Yes | 79 | apoptosis |
| 1 | TH | th | 15 | Yes | Yes | 77 | locomotion |
| 1 | CTNS | ctns | 11 | Yes | Yes | 77 | lifespan, development |
| 1 | CACNA1A | cacna1a b | 11 | Yes | Yes | 75 | locomotion |
| 2 | TSC2 | tsc2 | 9 | Yes | Yes | 74 | lifespan, locomotion |
| 2 | SLC39A14 | slc39a14 | 9 | Yes | Yes | 74 | locomotion |
| 2 | CHRNE | chrne | 13 | Yes | Yes | 71 | ephys |
| 2 | TSC1 | tsc1b | 13 | Yes | Yes | 64 | (tsc1a -development) |
| 2 | SLC30A10 | slc30a10 | 9 | Yes | Yes | 62 | manganese abnormal |
| 2 | GOT2 | got2a | 15 | Yes | Yes | 88 | – |
| 2 | QDPR | qdprb1 | 11 | Yes | Yes | 88 | – |
| 2 | PTS | pts | 11 | Yes | Yes | 85 | – |
| 2 | KCNT1 | kcnt1 | 10 | Yes | Yes | 85 | – |
| 2 | GRIN2A | grin2aa | 12 | Yes | Yes | 82 | – |
| 2 | COQ4 | coq4 | 15 | Yes | Yes | 80 | – |
| 2 | SLC13A5 | slc13a5a | 14 | Yes | Yes | 78 | – |
| 2 | PNPO | pnpo | 11 | Yes | Yes | 78 | – |
| 2 | PLPBP | plpbp | 14 | Yes | Yes | 77 | – |
| 2 | KCNQ2 | kcnq2a | 7 | Yes | Yes | 74 | – |
| 2 | SLC46A1 | slc46a1 | 15 | Yes | Yes | 69 | – |
| 2 | SLC52A3 | slc52a3 | 15 | Yes | Yes | 68 | – |
| 3 | SCN1A | scn1lab | 10 | Yes | No | 87 | lifespan, movement |
| 3 | SCN3A | scn1lab | 9 | Yes | No | 87 | lifespan, movement |
| 3 | SLC39A4 | slc39a4 | 10 | Yes | Yes | 57 | – |
| 3 | PRRT2 | prrt2 | 7 | Yes | Yes | 52 | – |
| 3 | SLC52A2 | slc52a2 | 7 | Yes | Yes | 40 | – |

Additional target genes that could be used to “humanize” cells and/or animals can include but are not limited toTRPV1, GPR55, VDAC1, GPR18, GPR 119, 5HT1A, and TRPV2.

Exemplary target genes for use in C. elegans can include the human genes KCNQ2 (C. elegans kqt-3), SLC6A4 (C. elegans mod-5), daf-12 nuclear hormone receptor (NHR) (C. elegans daf-12), CNR1 (C. elegans npr-19) and/or CNR2 (C. elegans srbc-48). These target genes are described in more detail below.

In some embodiments, the target gene can be KCNQ2 (potassium voltage-gated channel subfamily Q member 2) gene, having the C. elegans ortholog kqt-3. The KCNQ2 gene is an important disease associated gene with three established disease associations (Epileptic encephalopathy, early infantile, 7; Myokymia; Seizures, benign neonatal — www.omim.org). The retigabine agonist of KCNQ2 is used to treat loss-of-function (LOF) variant activity in epilepsy patients (Gunthorpe et al. Epilepsia. 2012 Mar;53(3):412-24). Conversely gain-of-function (GOF) in KCNQ2 is also associated with epilepsy (Niday and Tzingounis. Neuroscientist. 2018 Aug;24(4):368-380). As a result, either an agonist or an antagonist of KCNQ2 are needed for treatment of KCNQ2-associated epilepsies. An exemplary recoded transgene sequence for KCNQ2 is provided by SEQ ID NO: 5.

In some embodiments, the target gene can be SLC6A4 (Solute Carrier Family 6 Member 4 gene, the C. elegans ortholog being mod-5. The human coding sequence SLC6A4 (Solute Carrier Family 6 Member 4) encodes an integral membrane protein that transports the neurotransmitter serotonin from synaptic spaces into presynaptic neurons, is an important gene of mental health, and is the target of Selective Serotonin Reuptake Inhibitors (SSRI) (Kortagere et al. Neuropharmacology. 2013 Sep;72:282-90). Patients with genetic defects in SLC6A4 are also associated with Anxiety-related personality traits and Obsessive-compulsive disorder (see website for OMIN) and SSRIs are commonly used to treat major depressive disorder and anxiety. However, many unpleasant side-effects can occur and new SSRIs can be discovered using the provided SLC6A4 overexpressing transgenic line.

In some embodiments, the target gene can be daf-12, preferably a chimera therewith, the C. elegans daf-12 ortholog being a nuclear hormone receptor such as Vitamin D Receptor (VDR), Estrogen Receptor (ESR1), or Pregnane X Receptor (NR1l2). Chimeric proteins made with the daf-12 nuclear hormone receptor (NHR) are used for discovery of agonist and antagonist of human NHRs. Chimeras in daf-12 are used to detect ligands of human nuclear hormone receptors (NHRs). Nuclear hormone receptors are common targets for drug discovery as their function can often be modulated by small molecules, accounting for the therapeutic effect of 16% of all small-molecule drugs (Santos et al. Nature reviews: Drug discovery, Jan; 16(1): 19-34 (2017)). Both antagonist and agonist ligands of NHRs are involved in treating cancer (Safe et al. Mol Endocrinol. 2014 Feb;28(2):157-72) and environmental toxicity (Ren and Gao. Environ Sci Process Impacts. 2013 Apr;15(4):702-8). To create a platform specific to human NHR signaling pathway activation, a chimera can be made by fusing the DNA binding domain of daf-12 to the ligand binding domain of various drug and toxicity targets. Important targets for creation of DAF-12 chimeras are NHRs involved in hormonal signaling (VDR, RXRA, ESR1, PPARG,) and genes involved in biosensing environmental chemicals (AHR). Other NHR type for drug and toxicity discovery are RARA, RARB, RARG, PPARA, PPARD, NR1D1, NR1D2, RORA, RORB, RORC, NR1H3, NR1H2, NR1H4, NR1H5P, NR1l2, NR1l3, HNF4A, HNF4G, RXRB, RXRG, NR2C1, NR2C2, NR2E1, NR2E3, NR2F1, NR2F2, NR2F6, ESR2, ESRRA, ESRRB, ESRRG, NR3C1, NR3C2, PGR, AR, NR4A1, NR4A2, NR4A3, NR5A1, NR5A2, NR6A1, NR0B1, NR0B2. Measuring activity of DAF-12 chimeras is done through monitoring activity of the daf-12 gene. In C. elegans, the DAF-12 protein activity is a key regulator of development. When DAF-12 is activated by dafachronic acid under normal growth conditions, animals will proceed to their reproductive life stage. However, when animals face unfavorable conditions, dafachronic acid is not produced and the dauer program is activated leading animals into a stress-resistant arrested state. The unfavorable conditions can be mimicked by treatment with a DAF-12 antagonist, dafadine. Dafadine treatment overcomes the growth condition signal and animals enter the arrested state. Reporter lines can be used to monitor DAF-12 chimera activity. In order to create reporter lines for agonist and antagonist discovery, transcriptional response to treatment with dafachronic acid or dafadine was determined. Candidates for agonist reporters are genes that are strongly upregulated in the treated animals versus were non-treated controls. Candidates for antagonist reporters are genes that are strongly upregulated in the treated animals versus the non-treated controls.

In some embodiments, the target gene can be a guanine nucleotide binding protein-coupled receptor (GPCR). GPCRs are the targets of 34% of FDA-approved drugs (Hauser et al. Cell. 2018 Jan 11; 172(1-2): 41-54), approximately half being agonists of GPCR signaling activity. Exemplary GPCR target genes that can be used as described herein, and their nematode orthologs, are described in Tables 3 and 4.

TABLE 3

GPCR Targets with at least One Drug at Market

| AGONIST | | | | ANTAGONIST | | | |
|---|---|---|---|---|---|---|---|
| Human Target Gene | Nematode Ortholog | Sequence similarity (%) | Nematode LOF Phenotype | human | Nematode Ortholog | Sequence similarity (%) | Nematode LOF Phenotype |
| GABBR1 | gbb-1 | 59 | development | HTR1A | ser-4 | 52 | movement |
| GABBR2 | gbb-2 | 53 | behavior | ADRA2A | octr-1 | 52 | movement |
| ADRA2A | octr-1 | 52 | movement | HTR7 | ser-7 | 51 | movement |
| HTR1A | ser-4 | 50 | movement | ADORA1 | ador-1 | 51 | n.d. |
| ADORA1 | ador-1 | 48 | n.d. | GNRHR | gnrr-1 | 49 | n.d. |
| AVPR2 | ntr-1 | 48 | behavior | AVPR1A | ntr-1 | 49 | behavior |
| CHRM1 | gar-3 | 47 | movement | HTR2C | ser-1 | 48 | movement |
| LHCGR | fshr-1 | 46 | lethal | HTR2B | ser-1 | 48 | movement |
| CNR1 | npr-19 | 46 | n.d. | AVPR2 | ntr-1 | 48 | behavior |
| CNR2 | srbc-1 | 46 | development | CHRM1 | gar-3 | 47 | movement |
| CALCR | pdfr-1 | 45 | movement | HRH2 | ser-5 | 45 | movement |
| CRHR1 | seb-3 | 45 | n.d. | DRD1 | dop-1 | 44 | movement |
| HRH2 | ser-5 | 45 | movement | ADRA1B | dop-4 | 44 | movement |
| DRD1 | dop-1 | 44 | movement | ADORA2 B | ador-1 | 43 | n.d. |
| ADRA1B | dop-4 | 44 | movement | HRH1 | tyra-3 | 41 | movement |
| MC2R | srsx-24 | 44 | n.d. | P2RY12 | npr-8 | 39 | movement |
| ADORA2B | ador-1 | 43 | n.d. | DRD3 | dop-3 | 38 | movement |
| PTGER2 | srx-28 | 43 | n.d. | | | | |
| CCKAR | ckr-2 | 42 | development | | | | |
| PTGFR | srg-14 | 41 | n.d. | | | | |
| PTGER4 | srx-33 | 41 | n.d. | | | | |
| HRH1 | tyra-3 | 41 | movement | | | | |
| S1PR1 | C01F1.4 | 37 | nd | | | | |
| GNRHR | gnrr-1 | 37 | n.d. | | | | |
| GPR35 | gnrr-4 | 33 | erUPR | | | | |
| PTGIR | srx-28 | 31 | n.d. | | | | |
| DRD3 | dop-2 | 29 | movement | | | | |

TABLE 4

GPCR targets in clinical trials

| AGONIST | | | | ANTAGONIST | | | |
|---|---|---|---|---|---|---|---|
| Human Target Gene | Nematode Ortholog | Sequence similarity (%) | Nematode LOF Phenotype | human | Nematode Ortholog | Sequence similarity (%) | Nematode LOF Phenotype |
| GRM3 | mgl-1 | 57 | development | TACR3 | tkr-2 | 58 | n.d. |
| NPY2R | npr-6 | 55 | fecundity | GRM3 | mgl-1 | 57 | development |
| HTR7 | ser-7 | 51 | movement | FZD7 | mom-5 | 55 | lethal |
| SSTR2 | npr-24 | 49 | n.d. | FZD8 | cfz-2 | 53 | morphology |
| AVPR1 A | ntr-1 | 49 | movement | SSTR2 | npr-24 | 49 | n.d. |
| HTR2B | ser-1 | 48 | movement | CNR1 | npr-19 | 49 | n.d. |
| HTR2C | ser-1 | 48 | movement | CNR2 | srbc-1 | 46 | development |
| PPYR1 | npr-11 | 47 | movement | CRHR1 | seb-3 | 45 | n.d. |
| LHCGR | fshr-1 | 46 | lethal | CALCR L | seb-2 | 45 | n.d. |
| CALCR L | seb-2 | 45 | n.d. | CYSLT R1 | npr-14 | 45 | n.d. |
| F2R | ser-6 | 44 | movement | FFAR2 | srbc-55 | 44 | n.d. |
| CRHR2 | seb-3 | 43 | n.d. | P2RY1 | npr-33 | 42 | n.d. |
| MC5R | srsx-24 | 43 | n.d. | AGTR2 | npr-32 | 42 | n.d. |
| GHSR | nmur-3 | 42 | n.d. | CCKAR | ckr-2 | 42 | development |
| AGTR2 | npr-32 | 42 | n.d. | PTGER 4 | srx-33 | 41 | n.d. |
| F2RL2 | npr-25 | 42 | movement | PTGFR | srg-14 | 41 | n.d. |
| CXCR4 | npr-32 | 40 | n.d. | CCR2 | npr-32 | 41 | n.d. |
| MC3R | srsx-24 | 40 | n.d. | CXCR4 | npr-32 | 40 | n.d. |
| MC4R | srx-35 | 39 | n.d. | GPR84 | npr-23 | 40 | n.d. |
| MAS1 | srx-65 | 39 | n.d. | LPAR4 | gnrr-4 | 40 | erUPR |
| FFAR1 | ser-6 | 38 | movement | P2RY4 | npr-28 | 38 | n.d. |
| F2RL3 | gnrr-4 | 37 | erUPR | LTB4R | npr-23 | 38 | n.d. |
| GPR55 | gnrr-4 | 32 | erUPR | LPAR6 | gnrr-4 | 37 | erUPR |
| | | | | S1PR1 | C01 F1.4 | 37 | n.d. |
| | | | | P2RY10 | npr-15 | 36 | development |
| | | | | P2RY8 | gnrr-4 | 34 | erUPR |
| | | | | F2R | gnrr-4 | 34 | erUPR |
| | | | | PTGDR | srx-28 | 33 | n.d. |

For example, the HTR1A gene, the nematode ortholog being ser-4, is associated with mood disorders (Garcia-Garcia et al. Psychopharmacology (Berl). 231(4):623-36 (2014)) and periodic fever that is menstrual cycle dependent. See website for OMIN. Rescue of movement defects can be used to confirm the human HTR1A transgene functions in a similar manner as the ser-4 ortholog. In another example, the HTR7 gene, having the nematode ortholog ser-7, is linked to depression (Hedlund. Psychopharmacology (Berl). 206(3):345-54 (2009)). The nematode ortholog ser-7 promoter is primarily expressed in pharyngeal neurons (Hobson et al. Genetics. 172(1):159-69 (2006)), and rescue of pharyngeal movement defects can be used to confirm the human HTR7 transgene functions in a manner as the ser-7 ortholog.

In some embodiments, cannabidiol is the therapeutic target; that is, this target is affected by the cannabinoid receptors target genes CNR1 and CNR2 (cannabinoid receptor 1 and cannabinoid receptor 2, respectively), both known to be GPCRs. Multiple lines of anecdotal evidence attest to the therapeutic benefit of cannabidiol (CBD)-rich cannabis extracts (Gerard, et al. Biochem. J. 279 (1) 129-134 (1991); Munro, et al. Nature 365: 61-65, 1993); Thomas, et al. Br. J. Pharmacol. 150(5):613-23 (2007)). In humans, CNR1 is primarily expressed in the terminals of central and peripheral neurons while CNR2 is primarily expressed in immune cells both within and outside the central nervous system. However, CBD has a low affinity for binding CNR1 and CNR2, and studies suggest that additional targets may be relevant including TRPV1, GPR55, VDAC1, GPR18, GPR 119, 5HT1A, and TRPV2 (Gaston, et al. Curr. Neurol. Neurosci. Rep. 18(11):73 (2018); and Noreen, et al. Crit. Rev. Eukaryot. Gene Expr. 28(1):73-86 (2018)). Common CNR1 variants include Ala419Gly, Glu93Lys, Val2851le, Lys34Arg, and Val23Met. Common CNR2 variants include Gln63Arg, His316Tyr, Leu133l1e, Arg66Gln, and Ala280Val. In some embodiments, these target genes can be used as described herein, alone or in combination.

Kinase inhibitors are a large category of gene for therapeutic utility in cancer and other diseases. Table 5 lists exemplary protein kinase-related disease target genes that could be used as described herein. It is noted that more than half these potential human target genes have less than 50% similarity to the corresponding nematode ortholog (sequence similarity below 50%).

TABLE 5

Potential Kinase Targets

| Human Kinase Gene | Associated Disease | Nematode Orhtolog | Sequence Similarity (%) | Nematode Pheonotype |
|---|---|---|---|---|
| CDK5 | Lissencephaly 7 with cerebellar hypoplasia | cdk-5 | 88 | lethal |
| AURKC | permatogenic failure 5 | air-2 | 73 | lethal |
| AURKA | Colon cancer, susceptibility to | air-2 | 71 | lethal |
| MAP2K1 | Cardiofaciocutaneous syndrome 3 | mek-2 | 70 | lethal |
| MAP2K2 | Cardiofaciocutaneous syndrome 4 | mek-2 | 70 | lethal |
| SRC | Thrombocytopenia 6; Colon cancer, advanced, somatic | src-1 | 69 | lethal |
| MET | Deafness, autosomal recessive; Hepatocellular carcinoma, childhood type, somatic; Renal cell carcinoma, papillary, 1, familial and somatic; Osteofibrous dysplasia, susceptibility to | F11E6.8 | 64 | n.d. |
| MTOR | Focal cortical dysplasia, type II, somatic; Smith-Kingsmore syndrome | let-363 | 52 | lethal |
| BRAF | Adenocarcinoma of lung, somatic; Cardiofaciocutaneous syndrome; Colorectal cancer, somatic; LEOPARD syndrome 3; Melanoma, malignant, somatic; Nonsmall cell lung cancer, somatic; Noonan syndrome 7 | lin-45 | 51 | lethal |
| JAK3 | SCID, autosomal recessive, T-negative/B-positive type | src-1 | 49 | lethal |
| ABL1 | Congenital heart defects and skeletal malformations syndrome; Leukemia, Philadelphia chromosome-positive, resistant to imatinib | abl-1 | 49 | morphology |
| PI K3CA | Breast cancer, somatic; CLAPO syndrome, somatic; CLOVE syndrome, somatic; Colorectal cancer, somatic; Cowden syndrome 5; Gastric cancer, somatic; Hepatocellular carcinoma, somatic; eratosis, seborrheic, somatic; Macrodactyly, somatic; Megalencephaly-capillary malformation-polymicrogyria syndrome, somatic; Nevus, epidermal, somatic; Nonsmall cell lung cancer, somatic; Ovarian cancer, somatic | age-1 | 48 | fecundity |
| EGFR | Inflammatory skin and bowel disease, neonatal; Adenocarcinoma of lung, response to tyrosine kinase inhibitor in; Nonsmall cell lung cancer, response to tyrosine kinase inhibitor in; Nonsmall cell lung cancer, susceptibility to | let-23 | 44 | lethal |
| KIT | Gastrointestinal stromal tumor, familial; Germ cell tumors, somatic; Leukemia, acute myeloid; Mastocytosis, cutaneous; Mastocytosis, systemic, somatic | ver-3 | 43 | n.d. |
| ERBB2 | Adenocarcinoma of lung, somatic; Gastric cancer, somatic; Glioblastoma, somatic; Ovarian cancer, somatic | let-23 | 41 | lethal |
| JAK2 | Erythrocytosis, somatic; Leukemia, acute myeloid, somatic; Myelofibrosis, somatic; Polycythemia vera, somatic; Thrombocythemia 3; Budd-Chiari syndrome, somatic | src-1 | 40 | lethal |
| ALK | Neuroblastoma, susceptibility to, 3 | scd-2 | 40 | |
| PDGFRB | Basal ganglia calcification, idiopathic, 4; Kosaki overgrowth syndrome; Myeloproliferative disorder with eosinophilia; Myofibromatosis, infantile, 1; Premature aging syndrome, Penttinen type | ver-4 | 39 | movement |
| RET | Central hypoventilation syndrome, congenital; Medullary thyroid carcinoma; Multiple endocrine neoplasia; Multiple endocrine neoplasia IIB; Pheochromocytoma; Hirschsprung disease, protection against; Hirschsprung disease, susceptibility to, 1} | hir-1 | 39 | n.d. |
| CSF1R | Leukoencephalopathy, diffuse hereditary, with spheroids | ver-1 | 39 | n.d. |
| FLT3 | Leukemia, acute lymphoblastic, somatic; eukemia, acute myeloid, reduced survival in, somatic; Leukemia, acute myeloid, somatic | ver-3 | 39 | n.d. |
| KDR | Hemangioma, capillary infantile, somatic; Hemangioma, capillary infantile, susceptibility to | ver-4 | 37 | movement |
| PDGFRA | Gastrointestinal stromal tumor, somatic; Hypereosinophilic syndrome, idiopathic, resistant to imatinib | ver-4 | 37 | movement |

Nuclear hormone receptors are common targets for drug discovery as their function can often be modulated by small molecules, accounting for the therapeutic effect of 16% of all small-molecule drugs (Santos et al. Nature reviews: Drug discovery, Jan; 16(1): 19-34 (2017)). Both antagonist and agonist ligands of NHRs are involved in treating cancer (Safe et al. Mol Endocrinol. 2014 Feb;28(2):157-72) and environmental toxicity (Ren and Gao. Environ Sci Process Impacts. 2013 Apr;15(4):702-8). To create a platform specific to human NHR signaling pathway activation, a chimera is made by fusing the DNA binding domain of daf-12 to the ligand binding domain of various drug and cytotoxic agents. Important targets for creation of daf-12 chimeras are NHRs involved in hormonal signaling (VDR, RXRA, ESR1, PPARG,) and genes involved in biosensing environmental chemicals (AHR). Other NHR type for drug and toxicity discovery are RARA, RARB, RARG, PPARA, PPARD, NR1D1, NR1D2, RORA, RORB, RORC, NR1H3, NR1H2, NR1H4, NR1H5P, NR1l2, NR1l3, HNF4A, HNF4G, RXRB, RXRG, NR2C1, NR2C2, NR2E1, NR2E3, NR2F1 ,NR2F2, NR2F6, ESR2, ESRRA, ESRRB, ESRRG, NR3C1, NR3C2, PGR, AR, NR4A1, NR4A2, NR4A3, NR5A1, NR5A2, NR6A1, NR0B1, and/or NR0B2.

As another example, in some embodiments, human cytochrome p450 (CYP450) modified cells and/or transgenic animals (C. elegans, zebrafish) can be prepared to better understand drug efficacy and side-effects relating thereto. Exemplary CYP450s can include, for instance, constitutive CYP450, CYP2A6, CYP2B6, CYP2C9, CYP2C19, CYP2D6, CYP2E1, CYP3A4, and/or CYP3A5, among others. In some embodiments, as for other target genes discussed herein, the native CYP450 can be deleted and/or mutated. For example, since CYP3A5 catabolism of tacrolimus is required for its function as an immunosuppressant, whereas Tylenol® is metabolized by CYP2E1 to NAPQI, a reactive metabolize that targets mitochondria). Such cells and/or animals modified in such manner(s) can be combine with others to use in the drug discovery (e.g., combined with hSTXBP1 modified cells and/or animals).

As mentioned above, in some embodiments, this disclosure provides cells and/or animals can be engineered to express a therapeutic target gene and a mutated target gene (e.g., “double humanized patient allele models”). Exemplary combinations of a therapeutic target gene and a mutated target gene that can be used are shown in Table 6. Other combinations are also contemplated by this disclosure as will be understood by those of ordinary skill in the art.

TABLE 6

Exemplary Combinations for Double Humanized Patient Allele Model

| | |
|---|---|
| Therapeutic Target Gene | Human Disease Gene |
| SCN1A | SCN1A |
| KCNQ2 | KCNQ2 |
| GABAT | CDKL5 |
| SV2A | SCN2A |
| CACNA1G | STXBP1 |
| GRIA1 | SLC2A1 |
| GABRA1 | GABRG2 |
| GABRB1 | SCN8A |
| CACNA2D1 | UBE3A |
| KCNQ3 | FOXG1 |

### Phenotypic Assay Systems

In some embodiments, the phenotypic change in C. elegans animals can be determined by electrophysiology and/or the “food race assay”. Electrophysiology can be tested in a ScreenChip™ assay, which monitors the electrophysiology of pharyngeal pumping as individual animals enter a microfluidic channel (see, e.g., U.S. Pat. No. 9,723,817). The electrophysiology data from a ScreenChip™ is similar to an electrocardiogram signal, and depolarization and repolarization cycles of the pharynx food-pumping organ create a dominant and rhythmic contributor to the electrophysiology signal. Various sodium, potassium and calcium ion channels are major contributors to the observed electrical flux. Additional contributors are various ATP-driven ion pumps, and presynaptic inputs have a neuromodulatory effect on rhythmic pumping behavior. Test animals are introduced into the sensor region of the ScreenChip™ microfludics chamber, and 120 second recording of electrophysiology is made. On average, multiple animals are assayed for increased statistical power (typically n = or > 15), and the average behavior of a given parameter plot. For instance, loss of presynaptic unc-18 which is needed for coordinated neurotransmitter release results in a decreased pumping frequency when unc-18 is absent from the animal. In an exemplary embodiment, transgenic C. elegans animals can be generated by amino acid swap at particular positions in the humanized STXBP1 (e.g., R292H and/or R406H; or R388X) and tested by ScreenChip™ for alterations in pumping dynamics. In some embodiments, as in the examples described herein, the ScreenChip™ assay showed an increase in pumping frequency for the R292H and R406H variants and a decrease in pumping frequency in the R388X variant. The food race assay detects the capacity of the test transgenic nematode to exhibit coordinated movement in efforts to perform chemotaxis towards a food source. Typically, the assay can be performed in one hour wherein most of the control animals have reached the food, but test transgenic nematodes comprising clinical variants sequences defective in coordinated movement will not have reached the food. For example, in some embodiments, variants prepared by amino acid swap in the humanized locus can exhibit a strong phenotypic deficiency in the food race assay when compared to the wildtype STXBP1 gene swap line. In other embodiments, transgenic C. elegans comprising SEQ ID NO. 12 (CNR1) and/or SEQ ID NO. 14 (CNR2) can be used to test cannabinoids to see if the phenotypic effects of the variants on the animals are (e.g., as shown in the examples using the food race assay, the hSTXBP1 R406H containing C. elegans reaches the food more quickly with the cannabinoids than without). Each of these assays, along with others that show a phenotypic difference between the variants and the wildtype, can be used, alone or in combination.

Thus, in some embodiments, this disclosure provides methods for screening therapeutic agents to treat altered function of a human clinical variant, comprising: a) identifying a first test compound from a collection of compounds by in silico molecular dynamic simulation of the interaction of members of said collection of compounds with a test protein encoded by a mutated target gene, wherein a compound that interacts with the test protein is a first test compound; b) incubating a cell or animal with one or more first test compounds, wherein the cell or animal expresses the at least one mutated target gene, optionally optimized for expression in the cell or animal; the mutated target gene induces a modified phenotype in the cell or animal that differs from a normal phenotype induced by expression of a non-mutated version of the mutated target gene in the cell or animal; and, the modified phenotype results in at least a statistically significant measurable difference (e.g., about any of 5, 10, 15, 20, 25, 30, 35, 40, 45, 50, 55, 60, 65, 70, 75, 80, 85, 90, 95, 100, 150, 200, 250, 300, 350, 400, 450, 500, or more percent, and statistically significant) from the normal phenotype; and, identifying second test compound(s) that transform the modified phenotype of the cell or animal into a normal phenotype; and/or, c) incubating induced pluripotent stem cells (iPSCs) derived from a human patient expressing said mutated target gene therein with one or more first test compounds, and/or one or more second test compounds, wherein: the mutated target gene induces a modified phenotype in the iPSCs that differs from the normal phenotype induced by expression of a non-mutated version of the mutated target gene in iPSCs derived from a human patient that does not express the mutated target gene; and, the modified phenotype results in at least a statistically significant measurable difference (e.g., about any of 5, 10, 15, 20, 25, 30, 35, 40, 45, 50, 55, 60, 65, 70, 75, 80, 85, 90, 95, 100, 150, 200, 250, 300, 350, 400, 450, 500, or more percent, and statistically significant) from the normal phenotype; and, identifying third test compound(s) that transform the modified phenotype of the iPSCs into a normal phenotype as second test compound(s); and, optionally, d) repeating steps a) and b); a) and c); or, a), b) and c); to identify additional test compounds. In some embodiments, the mutated target gene comprises a human protein coding sequence. In some embodiments, the human clinical variant is classified as pathogenic or likely pathogenic. In some embodiments, the mutated target gene further comprises an inducible promoter operably linked to a reporter gene wherein the promoter is from a gene inhibited in response to expression of the human clinical variant, whereby therapeutic agents are identified when the inducible reporter gene is expressed, optionally wherein the reporter gene is a fluorescent or luminescent compound. In some embodiments, the phenotype is selected from electropharyngeogram variant, feeding behavior variant, defecation behavior variant, lifespan variant, electrotaxis variant, chemotaxis variant, thermotaxis variant, mechanosensation variant, movement variant, locomotion variant, pigmentation variant, embryonic development variant, organ system morphology variant, metabolism variant, fertility variant, dauer formation variant, stress response variant, and a combination thereof. In some embodiments, the phenotypic change is measured using a phenotypic assay selected from a measurement of electrophysiology of pharynx pumping, a food race, lifespan extension and contraction assay, movement assay, fecundity assay with egg lay or population expansion, apoptotic body formation, chemotaxis, lipid metabolism assay, body morphology changes, fluorescence changes, drug sensitivity and resistance assays, oxidative stress assay, ER stress assay, nuclear stress assay, response to vibration, response to electric shock, or a combination thereof. In some embodiments, the cell and/or animal further comprises an exogenous gene encoding a drug target gene, optionally wherein the drug target gene is a wild type gene. In some embodiments, this disclosure provides cells and/or transgenic non-human organism(s) comprising at least one of a coding sequence for mutated target gene, optionally operably linked to a promoter and/or optimized for expression in the non-human organism, wherein expression of the mutated target gene in the organism induces a modified phenotype therein; wherein the modified phenotype results in at least a statistically significant measurable difference (e.g., about any of 5, 10, 15, 20, 25, 30, 35, 40, 45, 50, 55, 60, 65, 70, 75, 80, 85, 90, 95, 100, 150, 200, 250, 300, 350, 400, 450, 500, or more percent, and statistically significant) difference from the normal phenotype expressed in the animal in the absence of expression of the mutated target gene. In some embodiments, the mutated target gene is a human gene. In some embodiments, the mutated target gene is selected from those listed in any of Tables 1-5 and/or a combination listed in Table 6, or a derivative thereof. In some embodiments, the cell and/or animal further comprises at least one exogenous wild-type gene different from the mutated target gene. In some embodiments, this disclosure provides a transgenic non-human animal (e.g., nematode, zebrafish) comprising at least one of a coding sequence for human CNR1 or CNR2 optimized for expression in the non-human organism, optionally wherein the coding sequence for human CNR1 or CNR2 is codon optimized, comprises intron sequences, absent of aberrant splice donor and acceptor sites; optionally comprises a constitutive promoter for expression CNR1 and CNR2 in neuron cells; optionally wherein said coding sequence is any of SEQ ID NOS. 12 or 14, respectively. In some embodiments, the transgenic organism further comprises at least one second heterologous human gene other than CNR1 or CNR2, optionally wherein the second heterologous human gene replaced a host nematode gene ortholog. In some embodiments, the coding sequences are integrated into the genome of the non-human organism and stably expressed. Other embodiments will also be apparent to those of ordinary skill in the art from this disclosure.

In some embodiments, this disclosure provides methods for screening therapeutic agents to treat altered function of a human clinical variant, the methods comprising identifying a first test compound from a collection of compounds by measuring the interaction of members of said collection of compounds with a test protein encoded by a mutated target gene, wherein a compound that interacts with the test protein is a first test compound; incubating a cell or animal with one or more first test compound(s), wherein the cell or animal expresses at least one mutated target gene, optionally optimized for expression in the cell or animal; the mutated target gene induces a modified phenotype in the cell or animal that differs from a normal phenotype induced by expression of a non-mutated version of the mutated target gene in the cell or animal; and, the modified phenotype results in at least a statistically significant measurable difference from the normal phenotype; and, identifying second test compound(s) that transform the modified phenotype of the cell or animal into a normal phenotype; and/or, optionally repeating steps a) and b) to identify additional test compounds. In some embodiments, the mutated target gene comprises a human protein coding sequence. In some embodiments, the human clinical variant is classified as pathogenic or likely pathogenic. In some embodiments, the mutated target gene further comprises an inducible promoter operably linked to a reporter gene wherein the promoter is from a gene inhibited in response to expression of the human clinical variant, whereby therapeutic agents are identified when the inducible reporter gene is expressed, optionally wherein the reporter gene is a fluorescent or luminescent compound. In some embodiments, the phenotypic assay is selected from a measurement of electrophysiology of pharynx pumping, a food race, lifespan extension and contraction assay, movement assay, fecundity assay with egg lay or population expansion, apoptotic body formation, chemotaxis, lipid metabolism assay, body morphology changes, fluorescence changes, drug sensitivity and resistance assays, oxidative stress assay, ER stress assay, nuclear stress assay, response to vibration, response to electric shock, or a combination thereof. In some embodiments, the phenotypic assay is a food race and/or measurement of electrophysiology of pharynx pumping. In some embodiments, the phenotype is selected from electropharyngeogram variant, feeding behavior variant, defecation behavior variant, lifespan variant, electrotaxis variant, chemotaxis variant, thermotaxis variant, mechanosensation variant, movement variant, locomotion variant, pigmentation variant, embryonic development variant, organ system morphology variant, metabolism variant, fertility variant, dauer formation variant, stress response variant, and a combination thereof. In some embodiments, the cell or animal further comprises an exogenous gene encoding a drug target gene, optionally wherein the drug target gene is a wild type gene.

In some embodiments, this disclosure provides transgenic non-human organisms comprising at least one of a coding sequence for mutated target gene, optionally operably linked to a promoter and/or optimized for expression in the non-human organism, wherein expression of the mutated target gene in the organism induces a modified phenotype therein; wherein the modified phenotype results in at least statistically significant measurable difference from the normal phenotype expressed in the animal in the absence of expression of the mutated target gene. In some embodiments, the mutated target gene is a human gene, optionally selected from the group consisting of any of those listed in Tables 1-5, and/or a combination of Table 6, or a derivative thereof. In some embodiments, the organism further comprises at least one exogenous wild-type gene different from the mutated target gene.

In some embodiments, this disclosure provides methods comprising introducing a mutated target gene into the genome of a non-human animal to produce a transgenic animal using CRISPR/Cas9-editing; identifying at least one induced mRNA the expression of which is induced by expression of the mutated target gene in the transgenic animal; operably linking a promoter region of the gene encoding the induced mRNA identified in step b) to a reporter gene to produce a biosensor; contacting a cell comprising the biosensor with a test compound; and, detecting expression of the reporter gene, wherein decreased expression of the reporter gene indicates the test compound is capable of suppressing the effect of the mutated target gene on expression of the induced mRNA. In some embodiments, the non-human animal is zebrafish or C. elegans. In some embodiments, this disclosure provides biosensors produced using such methods and/or a transgenic non-human animal comprising such a biosensor.

In some embodiments, this disclosure provides methods for screening therapeutic agents to treat altered function of a human clinical variant, comprising: identifying a first test compound from a collection of compounds by the interaction of members of said collection of compounds with a test protein encoded by a mutated target gene, wherein a compound that interacts with the test protein is a first test compound; incubating induced pluripotent stem cells (iPSCs) derived from a human patient expressing said mutated target gene therein with one or more first test compounds, and/or one or more second test compounds, wherein: the mutated target gene induces a modified phenotype in the iPSCs that differs from the normal phenotype induced by expression of a non-mutated version of the mutated target gene in iPSCs derived from a human patient that does not express the mutated target gene; and, the modified phenotype results in at least a statistically significant measurable difference from the normal phenotype; and, identifying third test compound(s) that transform the modified phenotype of the iPSCs into a normal phenotype as second test compound(s); and, optionally repeating steps a) and b) to identify additional test compounds. In some embodiments, the mutated target gene comprises a human protein coding sequence. In some embodiments, the human clinical variant is classified as pathogenic or likely pathogenic. In some embodiments, the mutated target gene further comprises an inducible promoter operably linked to a reporter gene wherein the promoter is from a gene inhibited in response to expression of the human clinical variant, whereby therapeutic agents are identified when the inducible reporter gene is expressed, optionally wherein the reporter gene is a fluorescent or luminescent compound. In some embodiments, the cell or animal further comprises an exogenous gene encoding a drug target gene, optionally wherein the drug target gene is a wild type gene.

In some embodiments, this disclosure provides pluripotent stem cells (iPSC) comprising at least one of a coding sequence for mutated target gene, optionally operably linked to a promoter and/or optimized for expression in the iPSC, wherein expression of the mutated target gene in the iPSC induces a modified phenotype therein; wherein the modified phenotype results in at least statistically significant measurable difference from the normal phenotype expressed in the iPSC in the absence of expression of the mutated target gene. In some embodiments, the mutated target gene is a human gene. In some embodiments, the mutated target gene is selected from the group consisting of any of those listed in Tables 1-5, and/or a combination of Table 6, or a derivative thereof. In some embodiments, the organism further comprises at least one exogenous wild-type gene different from the mutated target gene.

In some embodiments, this disclosure provides non-human transgenic organisms for assessing the interaction of a human therapeutic agent and a therapeutic target, the non-human transgenic host organism comprising a human heterologous gene encoding a therapeutic target sequence operably linked to a heterologous promoter selected for expression in the host organism cells, wherein: the human heterologous gene is inserted into a non-native locus of the host organism’s genome, and expression of the human heterologous gene is expressed in non-orthologous time and/or non-orthologous tissue. In some embodiments, the organism is a nematode (e.g., C. elgans) or zebrafish. In some embodiments, the therapeutic target is exogenously expressed as a single copy or multiple copies therein. In some embodiments, the therapeutic target comprises a clinical sequence variant, is a receptor, is a viral receptor, or is a G-protein coupled receptor associated with disease in humans. In some embodiments, the therapeutic agent comprises a compound, small molecule, and/or biologic component (e.g., mRNA). In some embodiments, the human heterologous gene comprises introns and exons.

In some embodiments, this disclosure provides methods for generating and/or assessing a non-human transgenic organism for assessing the interaction between a human therapeutic agent and a therapeutic target, wherein the transgenic organism has an increased sensitivity to the human therapeutic agent, the method comprising: selecting a target sequence comprising therapeutic target protein coding sequence and/or a long noncoding sequence; selecting a tissue-specific and/or time-specific regulatory sequence as a combination of a promoter sequence and a downstream untranslated region; combining the sequences by fusing the regulatory sequence to the target sequence; creating a non-human transgenic organism by inserting the combined sequence into a non-native locus of the genome of the non-human transgenic organism; and, optionally contacting the transgenic organism to the therapeutic agent and observing an elevated phenotypic response due to the activity of the transgene. In some embodiments, this disclosure provides methods for assessing the interaction between a human therapeutic agent and a therapeutic target over-expressed in a non-human transgenic organism for increased sensitivity of the host organism to the human therapeutic agent, the method comprising: providing a non-human transgenic organism of this disclosure comprising at least one of a human heterologous sequence expressing a therapeutic target providing a modified phenotype to the organism that is distinguished from a non-genetically modified host organism phenotype in at least one statistically significant measurable difference; contacting the genetically modified host organism of step a) with one or more human therapeutic agent(s) during an incubation period; performing one or more phenotype assay(s), during or after the incubation period, to assess interaction of the human therapeutic agent and overexpressed therapeutic target; and, recording a change in the modified phenotype following the phenotype assay, whereby, human therapeutic agents are assessed and selected based on their change in the modified phenotype. In some embodiments of such methods, expression of the therapeutic target in the non-human transgenic organism has a quantifiable phenotype that differs from the wildtype non-human organism. In some embodiments of such methods, the therapeutic target can be one previously identified via in-silico modeling, biochemical assay, or systems-level transcriptomic assay. In some embodiments of such methods, the phenotype can be measured as fluorescence of an exogenous reporting molecule, mRNA expression optionally assayed via RNAseq and/or microarray, the lifespan of the organism, or protein expression. In some embodiments of such methods, the phenotype can be revealed via response to exposure to an exogenously applied agent comprised of chemical enhancer or repressor, virus, bacterium, or pseudo- or chimeric virus. In some embodiments of such methods, exposure of the non-human transgenic animal to the therapeutic agent can result in an anti-correlated phenotype to the non-human organism expressing the therapeutic target, with or without exposure to the phenotype-revealing agent.

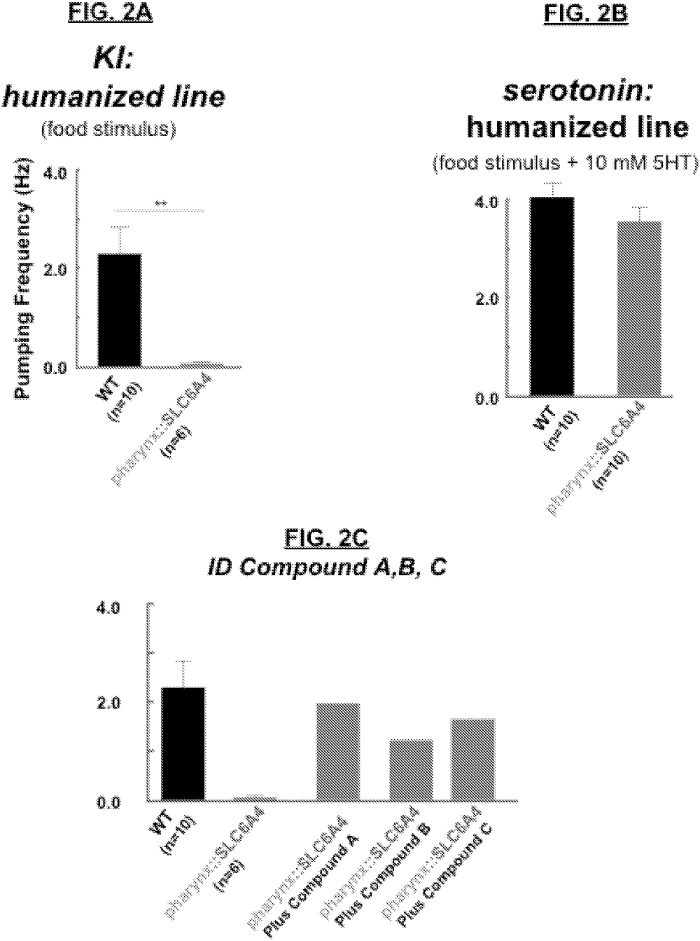

In some embodiments, this disclosure provides transgenic C. elegans lines for SLC6A4-association conditions as described in, e.g., Example 1 and FIG. 1 herein, including methods for using the same drug discovery for human therapeutic targets. In some embodiments, the therapeutic target gene of such C. elegans lines can first be optimized for expression in a host nematode and then incorporated into the genome of the organism. In some embodiments, the therapeutic target gene can be Human Solute Carrier Family 6 Member 4 (SLC6A4) (a serotonin transporter) as shown in, e.g., SEQ ID NOS. 1-4. Inhibitors of SLC6A4, selective serotonin reuptake inhibitors (SSRIs), are commonly used to treat major depressive disorder and anxiety but are also known to cause many unpleasant side-effects and can be discovered using the SLC6A4 overexpressing C. elegans transgenic nematodes and the methods disclosed herein. As shown in Example 1, C. elegans transgenic animals overexpressing human SLC6A4 in the pharyngeal muscle show a decrease in pumping frequency when stimulated to pump by food (a mild stimulus) as compared to the wildtype, and can be screened to identify novel SSRIs (see, e.g., FIGS. 2A-2C). An increase in pumping frequency in the SLC6A4 transgenic animals indicates that the drug is acting as an inhibitor to serotonin uptake. In some embodiments, and by way of example to demonstrate the general principle of modeling common genetic variants for pharmacogenetic studies, the humanized SLC6A4 C. elegans transgenic animals described above is injected with CRISPR/Cas9 components to create animals expressing SLC6A4 variants. In some embodiments, two common SLC6A4 variants, Gly56Ala and Lys605Asn, can be incorporated into the genome of a cell and/or animal using CRISPR/Cas9 for precise genome editing, and antagonists screened using variant-containing animals.

In some embodiments, this disclosure provides transgenic C. elegans for studying KCNQ2-association conditions as described in, e.g., Example 2 herein. KCNQ2 is an important human disease-associated gene associated with, for example, early infantile epileptic encephalopathy 7; Myokymia; and benign neonatal seizures. The retigabine agonist of KCNQ2 is used to treat loss-of-function (LOF) variant activity in epilepsy patients (Gunthorpe et al. Epilepsia. 53(3):412-24 (2012)). Conversely, gain-of-function (GOF) in KCNQ2 is also associated with epilepsy (Niday and Tzingounis. Neuroscientist. 24(4):368-380 (2018)). As a result, either an agonist or an antagonist of KCNQ2 are needed for treatment of KCNQ2-associated epilepsies. In some embodiments, the KCNQ2 gene can be gene-swapped into the kqt-1 locus of C. elegans animals. In preferred embodiments, such animals can be engineered for ectopic overexpression of KCNQ2 (e.g., SEQ ID NO: 5). In some embodiments, transgenic KCNQ2 C. elegans animals can be used for the detection of general antagonist activity with tissue-specific overexpression. In this embodiment, a snb-1 pan-neuronal promoter sequence (e.g., snb-1 promoter region (SEQ ID NO: 7) which can be inserted at the TTTTGCATCCGAAAAAGCGG (SEQ ID NO: 6) sgRNA site) can be inserted upstream of the humanized KCNQ2 gene start codon resulting in KCNQ2 overexpression in all neuronal cells, resulting in a greater sensitivity to transgene activity. The insertion of snb-1 promoter before the human coding sequence leads to hypermorphic over-expression of the human transgene in C. elegans, leading to severe attenuation of electrical signals due to excessive M-current activity and an animal with severely inhibited (suppressed) pharyngeal pumping activity. Exposure of an antagonist to the animal leads to restoration of near wild type activity. As a result, such an snb-1-enhanced KCNQ2 transgenic C. elegans animal can serve as a screening tool for finding KCNQ2 antagonists. In some embodiments, the snb-1-enhanced KCNQ2 line described above can modified to contain a clinical variant known to cause loss of function (LOF) using, e.g., CRISPR-mediated gene editing is used to install a LOF pathogenic allele (clinical variant) into the genome of the animals, creating an animal exhibiting a return towards normal pumping rates. In preferred embodiments, the addition of an agonist leads to a return to suppressed pumping rate. As such, the system with a LOF variant incorporated into the genome of a cell and/or animal can be used for highly personalized discovery of patient-specific agonist therapeutics. In some embodiments, the KCNQ2 humanized C. elegans cell line can be enhanced with a cell-specific ceh-2 promoter. The ceh-2 gene is primarily expressed in the M3 inhibitory neuron, whose activity is necessary for attenuating pharyngeal pump rates (Routhan et al. Dev Biol. 2007 Nov 1;311(1):185-99). To make a ceh-2-enhanced line, the ceh-2 promoter (e.g., SEQ ID NO: 9) can be inserted into the sgRNA site (e.g., SEQ ID NO: 8). In preferred embodiments, overexpression of KCNQ2 causes lower activity from the inhibitory neuron and greater than normal (hyperactive) pumping rate, which can be detected by EPG. Exposure to inhibitors of potassium channels can lead to restoration of normal EPG rates. As such, the ceh-1-enhanced KCNQ2 line can provide a screening tool for finding antagonist of KCNQ2. In some embodiments, a clinical variant causing gain of function (GOF) activity such as, e.g., a pathogenic allele (clinical variant) known to cause epilepsy can be incorporated into the genome of a cell and/or animal and/or into the ceh-2-enhanced KCNQ2 line, which can lead to a return towards normal pumping rates, because only partial and insufficient activity is expected from the clinical variant. When exposed to an antagonist, the animal activity returns to hyperactive pumping rates. As a result, the system with a GOF variant incorporated into the genome of a cell and/or animal can be used for highly personalized discovery of patient-specific antagonist therapeutics.

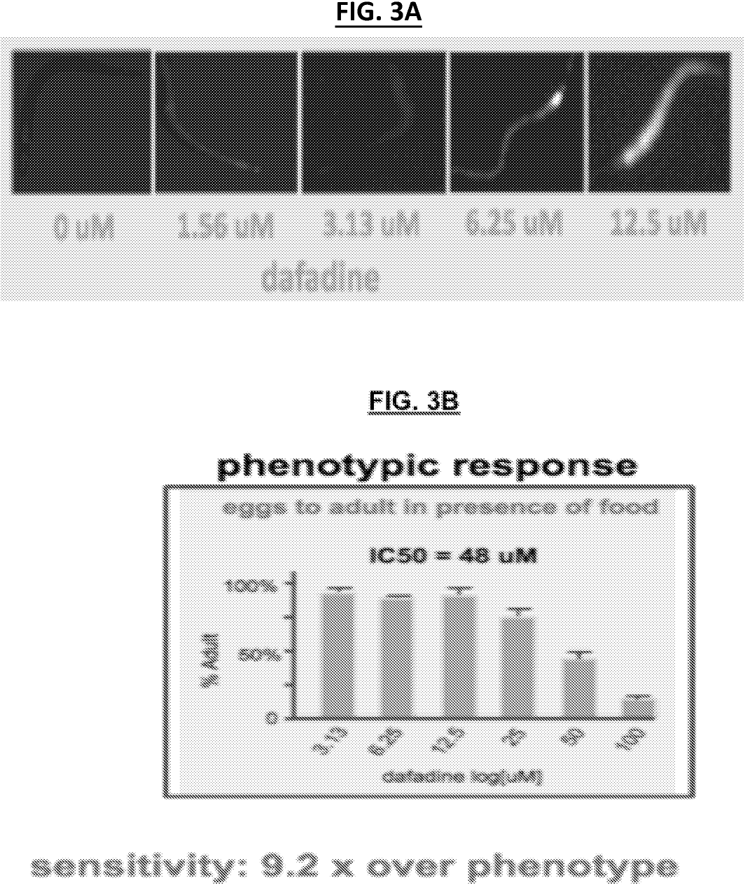

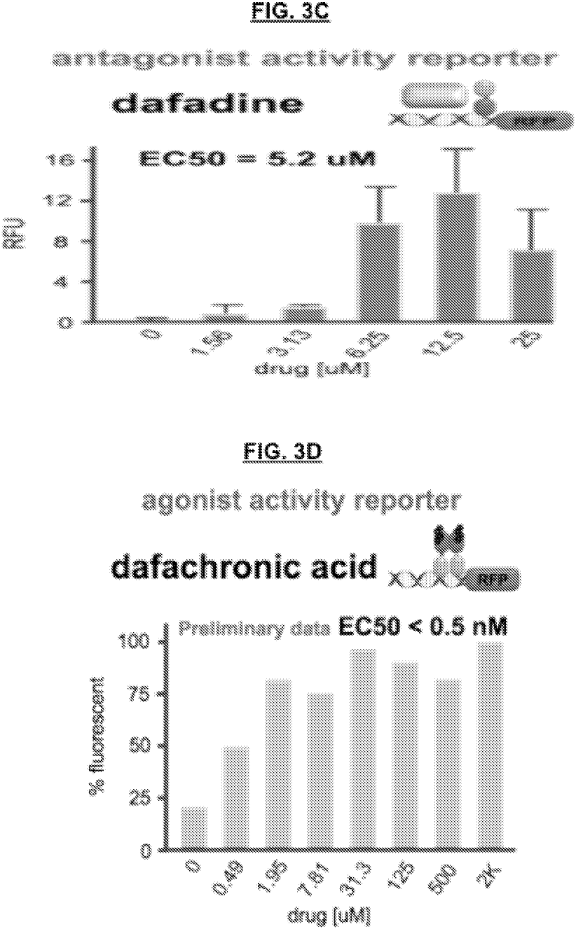

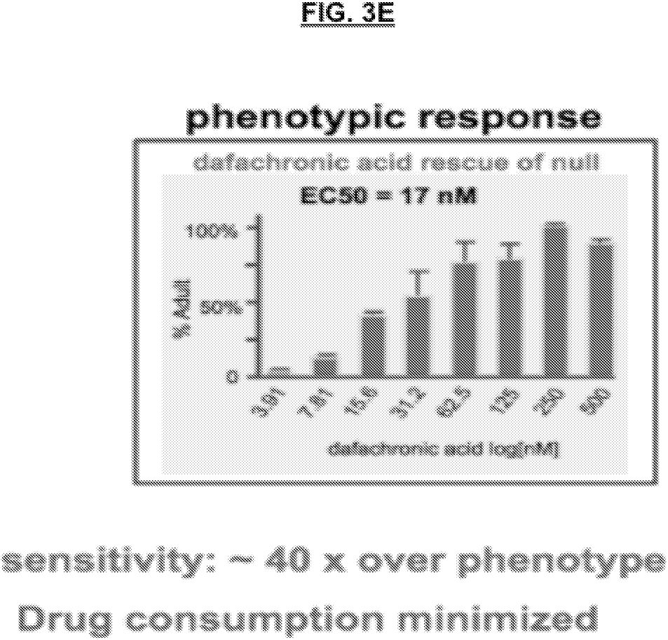

In some embodiments, DAF-12 transgenic C. elegans (daf-12) can be produced and used as reporter cell lines for agonist and antagonist discovery in nuclear hormone receptors as described in, e.g., Example 3 herein (see, e.g., FIGS. 3A-3E). To create a platform specific to human NHR signaling pathway activation, a chimera can be made by fusing the DNA binding domain of daf-12 to the ligand binding domain of various drug and cytotoxic agents. Nuclear hormone receptors are common targets for drug discovery as their function can often be modulated by small molecules, accounting for the therapeutic effect of 16% of all small-molecule drugs (Santos et al. Nature reviews: Drug discovery, Jan; 16(1): 19-34 (2017)). Both antagonist and agonist ligands of NHRs are involved in treating cancer (Safe et al. Mol Endocrinol. 2014 Feb;28(2):157-72) and environmental toxicity (Ren and Gao. Environ Sci Process Impacts. 2013 Apr;15(4):702-8). Important targets for creation of DAF-12 chimeras can include NHRs involved in hormonal signaling (e.g., VDR, RXRA, ESR1, PPARG) and genes involved in biosensing environmental chemicals (e.g., AHR). Other NHR type for drug and toxicity discovery are RARA, RARB, RARG, PPARA, PPARD, NR1D1, NR1D2, RORA, RORB, RORC, NR1H3, NR1H2, NR1H4, NR1H5P, NR1l2, NR1l3, HNF4A, HNF4G, RXRB, RXRG, NR2C1, NR2C2, NR2E1, NR2E3, NR2F1 ,NR2F2, NR2F6, ESR2, ESRRA, ESRRB, ESRRG, NR3C1, NR3C2, PGR, AR, NR4A1, NR4A2, NR4A3, NR5A1, NR5A2, NR6A1, NR0B1, and NR0B2. Chimeric proteins made with the daf-12 nuclear hormone receptor (NHR) can be used for discovery of agonist and antagonist of human NHRs and detect ligands of human NHRs. In C. elegans, the daf-12 protein activity is a key regulator of development. When daf-12 is activated by dafachronic acid under normal growth conditions, the animals will proceed to their reproductive life stage. However, when animals face unfavorable conditions, dafachronic acid is not produced and the dauer program is activated leading animals into a stress-resistant arrested state. The unfavorable conditions can be mimicked by treatment with a DAF-12 antagonist, dafadine. Dafadine treatment overcomes the growth condition signal and animals enter the arrested state. In some embodimetns, reporter lines produced as disclosed herein can be used to monitor daf-12 chimera activity. In order to create reporter lines for agonist and antagonist discovery, the transcriptional response to treatment with dafachronic acid or dafadine can be determined. Dauer animals can be treated with 500 nM dafachronic acid and harvested for RNA preparation after four hours of exposure, followed by analysis (e.g., RNA sequence analysis and/or qPCR). Candidates for agonist reporters can be genes that are strongly upregulated in the treated animals versus were non-treated controls.

In some embodiments, this disclosure provides GPCR transgenic C. elegans as described in, e.g., Example 4 herein. To create a platform system for detecting GPCR agonists, the human gene can first be inserted into the genome under the native promoter of the ortholog. When the gene is observed as capable of rescuing function, the promoter can be “bashed” by random mutagenesis to create downward attenuated expression . For instance, existing ChlP-seq data can be used to determine promoter binding elements region upstream of transgene start codon. CRISPR-mediated donor homology insertion with ODN templating can be used to alter codon composition in the promoter. Alternatively, non-templated CRISPR-mediated error-prone repair can be used. When a promoter-bashed animal starts to exhibit a phenotype similar to the KO allele, a drop in expression can be verified by rtPCR. An animal found exhibiting a loss-of-function (LOF) phenotype and a significant drop in transgene expression, the promoter-bashed system becomes a platform for discovery of agonists that restore behavior back to normal activity. Tables 3 and 4 above describe exemplary GPCR genes and their corresponding C. elegans orthologs. In some embodiments, the HTR1A gene can be inserted into the C. elegans genome as a gene-replacement of the coding sequence of orthologous ser-4. The HTR1A gene is associated with mood disorders (Garcia-Garcia et al. Psychopharmacology (Berl). 2014 Feb;231(4):623-36) and periodic fever that is menstrual cycle dependent. Rescue of movement defects can be used to confirm the human HTR1A transgene functions in a similar manner as the ser-4 ortholog. The ser-4 promoter can be bashed via CRISPR-based methods to randomly introduce changes that decrease hHTR1A expression. Concomitant LOF can be observed and movement defects return to the animal. As positive control(s), this transgenic strain can be exposed to a set of known FDA-approved agonists (cinitapride, ergoloid, flibanserin, lisuride, naratriptan, sumatriptan, vilazodone, vortioxetine, and zolmitriptan) to observe restoration of normal behavior, if any. The EC50 for each drug can be determined for capacity to restore behavior. As negative control, the strain cam be exposed to FDA-approved antagonist (acepromazine, alverine, asenapine, clozapine, iloperidone, lurasidone, methysergide, olanzapine, pipotiazine, thioproperazine and trazodone) and inability to rescue function is measured. Next, novel compounds in clinical trials for agonists can be exposed to the promoter bashed strain (eltoprazine, mn-305, opc-14523, pardoprunox, piclozotan, prx-00023, psn602, SEP-363856, sumatriptan, vibegron, xaliproden, and zolmitriptan) at the average EC50 concentration of known agonists. Good candidates for drug development can be revealed when candidate compounds are observed to cause restored behavior of the animal similar to the native-expressed hHTR1A strain. To create a GPCR antagonist detection system, the human gene can first be inserted into the genome under its gene ortholog’s native promoter. When the gene is observed as capable of rescuing function, heterologous promoter insertions can be used to upward attenuate expression. In C. elegans for instance, the eft-3 promoter is inserted to create higher expression of human transgene in all tissues. When this is too toxic to the animal, alternative promoters such as pan-neuronal snb-1 are used to get tissue-specific overexpression. When an overexpressed transgene system is observed to exhibit deviant behavior, the increase in expression is verified by rtPCR. When an animal is found exhibiting a gain of function phenotype and a significant increase in transgene expression, the promoter-bashed system becomes a platform for discovery of antagonists that restore behavior back to normal activity.

In some embodiments (see, e.g., Example 4 herein), the HTR7 gene can be inserted as gene-replacement of the coding sequence of orthologous ser-7. The HTR7 gene is linked to depression (Hedlund. Psychopharmacology (Berl). 2009 Oct;206(3):345-54). The ser-7 promoter primarily expresses in pharyngeal neurons (Hobson et al. Genetics. 2006 Jan;172(1):159-69). Rescue of pharyngeal movement defects can be used to confirm the human HTR7 transgene functions in a similar manner as the ser-7 ortholog. Next, the highly-expressing, pan-neuronal promoter, snb-1p, can be inserted upstream of the ser-7 start codon to induce hypermorphic expression in neuronal tissues. Concomitant GOF can be observed because movement defects from transgene overexpression occur in the animal. As positive control, the strain can be exposed to a set of known FDA-approved antagonists (asenapine, iloperidone, lurasidone, methysergide and olanzapine) to see by how much restoration of normal behavior can be achieved. The EC50 for each drug can be determined for capacity to restore behavior. As negative control, the strain can be exposed to FDA-approved agonist (vortioxetine) and inability to rescue function can be measured. Next, a novel compound in clinical trials for antagonist can be exposed to the promoter bashed strain (ly2590443) at the average EC50 concentration of known antagonists. In preferred embodiments, good candidates for drug development can be revealed when candidate compounds are observed to cause restored behavior of the animal similar to the native-expressed hHTR7 strain.

In some embodiments, this disclosure provides human cannabinoid receptor transgenic C. elegans as described in, e.g., Example 5 herein. The nucleic acid constructs used to transform C. elegans can comprise a promoter regulatory sequence for ubiquitous expression in C. elegans neurons, a human cannabinoid receptor optimized for expression in C. elegans, and a 3′UTR in a plasmid designed for insertion into the C. elegans genome. In some embodiments, a region of 1461bp immediately preceding the ATG of snb-1 neuronal promoter can be amplified from the C. elegans genome using PCR primers. In some embodiments, human cannabinoid receptor cDNAs (CNR1 (SEQ ID NO: 12) and CNR2 (SEQ ID NO: 14)) can be optimized for expression in C. elegans by optimizing cDNA codons optimal for expression in C. elegans using the C. elegans Codon Adapter (Redemann, et al. Nat Methods. 2011 Mar;8(3):250-23) and adding synthetic introns to the cDNA sequence (Hebsgaard, et al. Nucleic Acids Res., Vol. 24, No. 17, 3439-3452 (1996); Brunak, et al. J. Mol. Biol., 220, 49-65 (1991)); the appropriate Gibson ligation arms can then be added to the DNA sequence for cloning (Gibson et al. Nat. Methods May;6(5):343-5 (2009)).

The tbb-2 3′utr was used as a 3′ UTR that is permissive for expression. A region of 332bp immediately following the Stop Codon of tbb-2 was amplified from the C. elegans genome using primer sequence GCCCTCTAAgataaatgcaaaatcctttcaagcattccc (SEQ ID NO: 10) /tgagacttttttcttggcggca (SEQ ID NO: 11). The uppercase sequence was used for Gibson ligation and the lowercase is specific for amplification. The plasmid backbone for this construct contained elements for growth and selection in E. coli the ColE1 origin and Ampicillin resistance. In addition, they contained elements for integration into the worm genome. pNU2006 contains homology arms for integration into Chromosome II at ttTi5605 using the MosSCI method (Frøkjaer-Jensen et al. Nat Genet. 40(11): 1375-1383 (2008)). pNU2007 contains homology arms for integration into Chromosome IV at cxTi10882 using the MosSCI method. Both constructs also contain an unc-119 selection marker from C. briggsae to allow identification of transgenic animals. These parts were assembled together using Gibson ligation and bacterial transformation. Bacterial colonies were observed, and a subset were grown in a liquid culture overnight. The plasmids were purified, and diagnostic restriction digest was performed to see if the plasmid had the expected size digest products. A plasmid showing the expected banding pattern in the diagnostic restriction digest was then sent out for sequencing (Eton Bio). AB1 files were provided and analyzed to determine that no mutations that would affect function were present. In some embodiments, transgenic C. elegans comprising CNR1 (SEQ ID NO. 12) or CNR2 (SEQ ID NO. 14) and expressing SEQ ID NO. 13 or SEQ ID NO. 15, respectively, can be crossed together to generate a transgenic C. elegans comprising both SEQ ID NO. 12 (CNR1) and SEQ ID NO. 14 (CNR2). Males can be generated from one of the strains and crossed with hermaphrodites from the other strain.

In some embodiments, this disclosure provides a combination of C. elegans lines comprising CNR1 and/or CNR2 with other transgenes as described in, e.g., Example 6. In some such embodiments, the CNR1 and/or CNR2 transgenic C. elegans lines prepared as described herein can be genetically combined with other C. elegans strains (e.g., transgenic strains) to deplete the native endocannabinoid signaling pathway. In some embodiments, such other strains lack the endocannabinoid synthesis gene products such as phospholipase C beta (egl-8), diacylglycerol lipase (dagl-2), and N-acyl phosphatidylethanolamine-specific phospholipase-D (nape-1 and nape-2) (Harrison et al. PLoS One. 2014 Nov 25;9(11):e113007), and/or with strains lacking the native endocannabinoid receptors such as npr-19 (Oakes et al. J Neurosci. 37(11): 2859-2869 (2017)), strains with altered ability to impair endocannabinoids are used such as those depleted in or over-expressing the fatty acid amide hydrolase (faah-2), which degrades and inactivates endocannabinoids (Harrison et al. PLoS One. 9(11):e113007 (2014)). Depletion of the native endocannabinoid signaling pathway components could provide increased sensitivity to exogenous cannabinoid signaling through the humanized CNR1 and CNR2. In some embodiments, the CNR1 and/or CNR2 transgenic C. elegans lines prepared as described herein can be genetically combined with C. elegans strains comprising and expressing additional humanized proteins to identify cannabinoid treatment options for certain diseases. In one embodiment, human alleles are incorporated into the genome of a cell and/or animal in the nematode genome replacing a native allele for a gene with high homology, or in a different site of the genome, following standard methodologies (e.g., as described herein). In another instance, clinical variants of those human alleles are incorporated into the genome of a cell and/or animal into the human allele (e.g., STXBP1, KCNQ2, CACNB4, etc.) expressed in C. elegans, e.g., using genetic crosses of two different transgenic nematodes, and/or genome engineering. Such multi-component humanized lines can be used in drug screening for cannabinoid effects on ameliorating phenotypes of the pathogenic disease genes. Drug activity on the humanized lines can be compared to wildtype, null mutants, and the humanized controls with and without the CNR1 and CNR2 to determine if possible therapeutic effects are occurring. In some embodiments, the CNR1 and/or CNR2 transgenic C. elegans lines prepared as described herein can be genetically combined with C. elegans strains expressing the STXBP1 human protein variants, optimized for expression in the C. elegans, to identify cannabinoid treatment options for diseases such as epilepsy. In some embodiments, pathogenic variants (e.g., S42P, R406H, R292H, and R388X) can be introduced into the humanized STXBP1 line using a CRISPR/Cas9 system and can result in phenotypic differences when compared to the wildtype human STXBP1 expressed in C. elegans in movement and morphology assessments.

In some embodiments, this disclosure provides reagents and methods for use in Zebrafish as described in, e.g., Example 7. In some such embodiments, this disclosure provides exemplary configurations of the present nucleic acid constructs each comprising a human cannabinoid receptor, CNR1 and/or CNR2, for expression in zebrafish. In some embodiments, such constructs can include a promoter regulatory sequence for ubiquitous expression in Zebrafish neurons, human cannabinoid receptors optimized for expression in zebrafish, and a 3′UTR in a plasmid designed for insertion into the zebrafish genome. In some embodiments, the zebrafish neuronal promoter can be neurod1. In some embodiments, donor homology constructs can be designed using standard techniques such as by using Tol2 (Kawakami, et al. PNAS USA, 97, pp. 11403-11408 (2000)) for random insertion in the genome, and/or the CRISPR/Cas9 system for targeted insertion (Kimura, et al. Sci. Rep., 4: 1-7 (2014)). Targeted insertion can be directed to a “safe-harbor” site or to the native Zebrafish cnrl gene. An injection mix can be created with multiple nucleic acid and protein components, including the donor homology DNA and double-stranded break inducing component. Zebrafish embryos less than 4 hours post fertilization can be injected, incubated to adulthood, and tested for germline transmission of the transgene. Animals homozygous for the genome edit can be created by crossing using similar methodology using standard techniques (e.g., as described herein). Alternatively, point mutations modeling human variants can be incorporated into the genome of a cell and/or animal in the native Zebrafish cnrl or cnr2 genes. The resultant transgenic zebrafish can be used to screen for the effects of cannabinoids via phenotype read-out assays. Cannabinoid libraries can be generated de novo or purchased from commercial suppliers. Cannabinoids can be screened for synergistic or antagonistic activity toward alteration of normal phenotype using one or many phenotypic assays. In some embodiments, titrations at three (3)-fold doses (e.g., ranging from 1 mM to 128 mM) can be performed for each compound. After two hours of exposure, cohorts can be tested in one or more phenotypic assays, response curves plotted, and EC₅₀ values calculated. In some embodiments, such as for determining if antagonistic or synergistic activities are present between anti-epileptic drugs (AEDs) and cannabinoids, pairwise combinations can made followed by exposure of the humanized transgenic lines to such combinations. In mouse model assays (Smith, MD, Wilcox, KS, White, S. Analysis of Cannabidiol Interactions with Antiseizure Drugs. Annual Meeting American Epilepsy Society, 2015), carbamazepine (CBZ) was observed to antagonize a CBD, while levetiracetam (LEV) acts synergistically with CBD, and similar combinations can be tested in the transgenic zebrafish. In some embodiments, to observe antagonistic and synergistic effects, the concentration of CBD can be held at its observed EC₅₀ value in combination with titrations near the EC₅₀ of the AED drugs. In some embodiments, the phenotypic effect of CBZ can be suppressed while that of LEV enhanced. In other embodiments, other AED effects, if any, can be enhanced, suppressed, or remain unaltered.

In some embodiments, this disclosure provides transgenic C. elegans lines expressing therapeutic targets and disease genes as described in, e.g., Example 8. In some embodiments, such transgenic animals (e.g., nematodes such as C. elegans, or zebrafish) and methods of preparing and using same can be used to identify therapeutic targets, wherein in some embodiments the therapeutic target gene and disease gene can be first optimized for expression in a host animal and then incorporated into the genome of the animal. For instance, in some embodiments, the human coding sequence for gamma-aminobutyric acid transaminase (hGABAT, an important target for anti-epilepsy drugs such as valproic acid and vigabatrin even when a variant in hGABAT is not present in the treated patient) can be inserted into the C. elegans genome. Literature evidence can be found of the effectiveness of vigabatrin for patients with hSTXBP1 variants (Romaniello et al. 29(2): 249-253 (2013)), hKCNQ2 variants (Lee, et al. Pediatr. Neurol. 40(5):387-91 (2009)), and hCDKL5 variants (Melikishvili G, et al. Epilepsy Behav. 94:308-311 (2019)), among others. In some embodiments, the human hGABAT coding sequence is optimized for expression in C. elegans, artificial C. elegans introns can be added and aberrant splice sites removed (e.g., SEQ ID NO: 16). In some embodiments, the hGABAT sequence can be inserted into the C. elegans genome at the orthologous gene locus, gta-1, using CRISPR/Cas9. The hSTXBP1 sequence is inserted into the C. elegans genome at the orthologous gene locus, unc-18, using CRISPR/Cas9 using methods described in WO 2019/165128. The C. elegans genes gta-1 and unc-18 have overlapping tissue expression so native orthologous expression can be used, but in cases where the expression is not overlapping alternative promoters and transgenic methods are used. In some embodiments, the insertion of hGABAT can be combined with the insertion of hSTXBP1 to create a double humanized model. Pathogenic variants can be modeled in hSTXBP1 using CRISPR/Cas9 to create double humanized patient allele models. Movement, morphology, electrophysiology, growth rate, and other phenotypic measures can be used to characterize the double humanized patient allele models. Double humanized patient allele models can be treated with compounds targeting hGABAT and are characterized by phenotyping. Compounds that bring the phenotype of the double humanized patient allele models closer to the wild-type double humanized model can be considered potential hits. Those that do not improve the hSTXBP1 animal that does not contain the hGABAT or that has the native gta-1 also knocked out are candidates for directly targeting hGABAT. In some embodiments, a double humanized patient allele model can be developed by selecting one of the combinations of Therapeutic Target Gene and a different Human Disease Gene shown above in Table 6.

In some embodiments, this disclosure provides methods for in-silico compound screening (see, e.g., Example 9 herein). In some embodiments, the methods use a humanized C. elegans animal model combined with patient human induced pluripotent stem cells (hiPSC)-derived in a neuronal assay for rapid validation of in-silico drug screening data to identify candidate compounds with therapeutic potential. In some embodiments, the pathogenic R406H variant of STXBP1 (STXBP1(R406H)) can be studied. Syntaxin binding protein 1 (STXBP1) plays an important role in presynaptic vesicle docking and fusion, and thus an essential component in the neurotransmitter secretion mechanism (Toonen, et al. Trends Neurosci. 30: 564-72 (2007)). STXBP1 related disorders are collectively termed STXBP1-encephalopathy (STXBP1-E) and are neurodevelopmental disorders displaying diverse clinical features including epilepsy (~95% of patients), different movement disorders, intellectual disabilities (ID) and autistic features (Stamberger, et al. Neurology 86: 954-62 (2016)). Pathophysiology is caused by variations in STXBP1 gene, such variations including missense, nonsense, frameshift, and splice site changes, and/or whole gene deletions. Current treatment for STXBP1-E patients is limited to amelioration of symptoms (e.g., using physiotherapy, speech therapy, and/or occupational therapy) and seizure control, which is not fully effective for all patients and almost one in three remain therapy-resistant. The humanized C. elegans animal model to identify therapeutics that act directly to reverse the deficiencies caused by a pathogenic variation in STXBP1. In some embodiments, a nucleic acid encoding STXBP1(R406H) (i.e., the STXBP1(R406H) transgene) can be introduced into the C. elegans genome. One or more compounds (e.g., a library of compounds) can be screened to identify therapeutic candidates capable of restoring non-pathogenic (i.e., normal, wild-type) behavior. Assays that can be used in these comparisons can include but are not limited to the worm locomotion assay (Brenner, S., et al. Genetics 77: 71-94 (1974)) and/or the aldicarb sensitivity assay (Martin, et al. Curr. Biol. 21: 97-105 (2011)), among others. The aldicarb assay, for example, can be used to directly monitor the capacity of a test compound to restore evoked release of acetylcholine from presynaptic termini. Knock-out unc-18 C. elegans animals lacking their ortholog version of the STXBP1 gene are highly resistant to aldicarb-induced paralysis. Aldicarb acts by blocking acetylcholinesterase which leads to an overstimulation of the post-synaptic terminus. The loss of unc-18 prevents this over-stimulus because synaptic vesicle release is greatly reduced in the unc-18 null animals. The result is unc-18 animals are highly resistant to aldicarb overstimulation (i.e., stabilizing STXBP1). It has been shown that when trehalose was exposed to a strain containing the R406H variant, the levels of paralysis was observed to increase to wild-type levels (Guiberson, et al. Nat. Commun. 9: 3986 (2018)). In some embodiments, the methods disclosed herein can include performing in-vivo testing in STXBP1(R406H) humanized animal for activity on in silico derived hits of FDA approved compounds; determining if activity is conserved by screening the in-vivo validated and non-validated in silico hits for ability to improve neuronal function of iPSC-derived neurons from a STXBP1(R406H) patient; and, using in-vivo screening results to refine the in-silico approach and select more drug candidates for activity on R406H (i.e., a repetitive or iterative process) (e.g., as illustrated in FIG. 1). The exemplary in-vivo screening methods disclosed herein can be used to refine the in-silico approach and select more drug candidates for activity on R406H for testing back in-vivo. Hit expansion can be carried out in order to explore a wider chemical space, and the biological results used to identify key features of the potential hits, enabling the identification of compounds with a higher probability to have the desired therapeutic effect. Multiple rounds of discovery and refinement can be deployed to explore pharmacophore optimization. The potential validation of the in-silico/ in vivo/human-IPSC modeling pipeline for personalized drug repurposing disclosed herein for mutation R406H could be further used as a personalized approach that is cost-effective and mutation specific for other STXBP1 variants, thereby dramatically shortening the period between experimentally identifying a potential candidate drug and testing it clinically on STXBP1 patients.

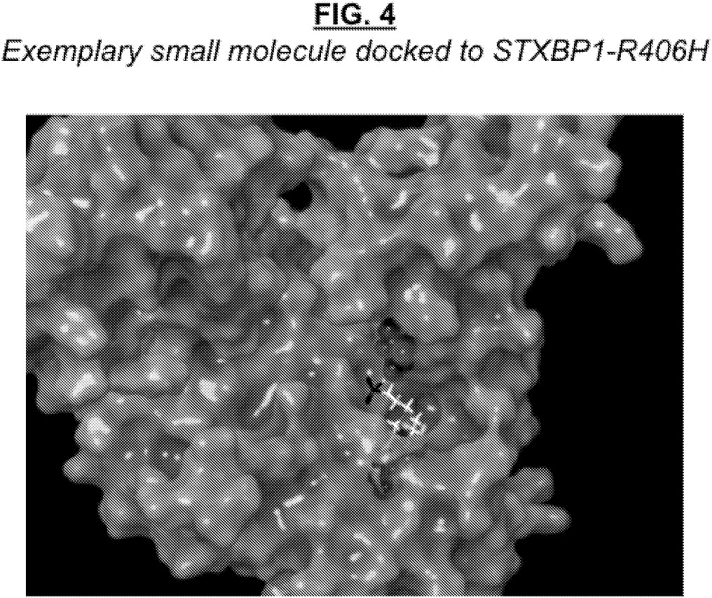

In some embodiments, this disclosure provides reagents and methods for in-silico Predictive Computer-aided drug design is a fast and efficient approach to drug discovery (Macalino, et al. Arch. Pharm. Res. 38: 1686-1701 (2015)) (see, e.g., Example 9 herein). In some embodiments, molecular dynamic simulations can be applied to predict the effect of the R406H mutation on structure and function of STXBP1 (see, e.g., FIG. 4). The high-resolution X-ray crystal structure (PDB: 4JEH) www.rcsb.org/structure/4JEH can be used as a starting point of the MD simulation. Analyzing the MD extracted trajectories, influence of the mutation on protein conformation and function is similar to that observed by Bar-On and colleagues (Bar-On, et al. PLoS Comput. Biol. 7: e1001097 (2011)). A cavity at the vicinity of residue 406 that is formed as a result of the mutation due to a missing hydrogen-bond was observed. It was hypothesized that binding a small molecule at that cavity may fill the gap created and rescue the protein function. An example of a small molecule docked to STXBP1-R406H is shown in FIG. 4. In some embodiments, a subset including 20 of approximately 100 candidates in silico-predicted drugs/natural products can be phenotypically tested on the R406H STXBP1 variant strain for capacity to restore normal behavior in the humanized C. elegans animals. In some embodiments, the process for making the humanized C. elegans animal model for the R406H STXBP1 variant strain can include three steps: 1) the native gene (unc-18) can be removed (i.e., deleted) from the animal genome or amino acid(s) can be changed at conserved positions in the nematodes’s unc-18 ortholog version of the STXBP1 gene (the “native locus”), resulting in a severely uncoordinated animal (i.e., pathogenic behavior, pathophysiology); 2) the removed DNA sequence can be replaced with the human STXBP1 gene sequence, which results in almost complete rescue-of-function; and, 3) the R406H variation can be inserted into the STXBP1-humanized locus after which the animal is characterized to identify any pathogenic behavior (e.g., a severely uncoordinated animal). Functional tests used can be high-dimensional phenotypic studies (e.g., electrophysiology, motion, morphology and gene expression) that allow quantitation of a variety of phenotypic deficiencies in transgenic animals. The functional testing of R406H in the STXBP-humanized locus preferably shows clear correlation to the human disease phenotype. The use of human sequences as a backbone for variant installations can sensitize the animal for detection of known pathogenic missense variants (STXBP1(R292H) and STXBP1(R406H)). Humanization can be accomplished by introducing amino acid at conserved positions in the nematodes’s unc-18 ortholog version of the STXBP1 gene (“Native locus”); or, expressing the entire human STXBP1 gene as a replacement of the C. elegans native unc-18 coding sequence.

In some embodiments (see, e.g., Example 9), a liquid aldicarb sensitivity assay can be performed by introducing transgenic animals to one or more compounds in 96-well format, followed by monitoring over time for aldicarb induced loss of motion by use of a WMmicrotracker Apparatus (Nemametrix, Inc). In some embodiments, three-fold titrations of compound starting at 100 mg/ml for 12 wells can be used to test a five (5) order-of-magnitude concentration range. Suspensions of animals can be introduced in parallel to the serial dilutions and the activity of the animals monitored across 20-minute intervals for two (2) hrs. Plots of sensitivity to aldicarb-induced paralysis can indicate which compounds are candidates for further analysis. Compounds of potential interest are those that re-sensitize the animals to aldicarb exposure. To filter out non-specific paralysis, hits found on the STXBP1(R406H) strain can then be rescreened on the STXBP1(WT) strain. Hits with strong ratio of R406H/WT activity can then advance to human iPSC cell activity detection.

As mentioned above, in some embodiments, induced pluripotent stem cell (iPSC) can be used to reprogram patient-derived somatic cells into an embryonic stem cell-like state followed by a differentiation to the disease-relevant cell type. This technology has proven to be a powerful tool for modeling diseases and for drug screening (Takahashi, et al. Cell 131: 861-872 (2007); Lee, et al. Nat. Biotechnol. 30: 1244-8 (2012)) and was previously been implemented in disorders similar to STXBP1 (e.g., Rett syndrome and Dravet syndrome (Marchetto, Cell, 143(4), 527-539 (2010); Schuster, et al. Neurobiology of Disease, 132, 104583 (2019)). The use of iPSC-derived neurons from R406H patient can provide a unique opportunity for testing either the in silico drug hits directly on the cells or after their previous validation by the transgenic C. elegans in vivo. In addition to testing for functional improvement of the iPSC-derived neurons, this tool can provide for exploring the potential rescue mechanism of positive hits and gaining a more thorough understanding of pathological mechanisms underlying the patient symptoms and improve patient treatment’s precision. Thus, producing viable and functional neurons from STXBP1(R406H) patient-derived iPSC can provide for screening of in silico-derived and/or C. elegans in-vivo validated hits and test the ability of compounds identified thereby to improve neuronal function/phenotype in the human cell model. In some embodiments, primary fibroblasts can be isolated from a skin biopsy of a human patient. Viable iPSC and isolated stem cell colonies will be selected based on genomic stability and pluripotential capacity. The specific capacity to produce functional cortical neurons can then be tested. Those cells identified as having such capacity can be used in functional neuronal assays to test the drugs/natural products by their potential to improve in vitro electrophysiological activities and neurotransmitters release, in a similar manner as previously used for phenotyping STXBP1 variants containing neurons (Yamashita, et al. Epilepsia 57: e81-e86 (2016); Kovačević, et al. Brain (2018)).

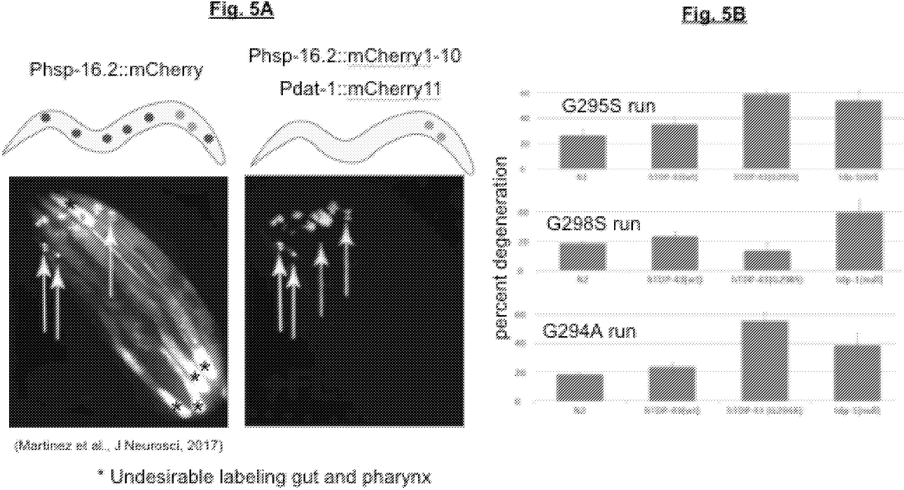

In some embodiments, this disclosure provides a drug screen platform for Alzheimer related dementias (see, e.g., Example 10 herein). In some such embodiments, this disclosure provides reagents and methods for identifying therapeutic compounds for reversing pathogenic behavior in genetic variants associated with Alzheimer Dementia Related Disorders (ADRD), which affects more than five million people in the US (1.5% of population). A variety of genes are involved in the defects that lead to neurodegeneration and shortened lifespan including but not limited to MAPT, GRN, TARDBP, APP, PSEN1, and PSEN2. Orthologs to these genes exist in C. elegans (ptl-1, pgrn-1, tdp-1, apl-1, sel-12, hop-1, respectively) and a variety of prior studies indicate defects in these genes influence neuronal integrity, elevate calcium signaling, and reduce lifespan in C. elegans (Chew et al., J Cell Sci, 2013; Salazar et al., J Neurosci, 2015; Ewald et al., Aging Cell, 2016; Sarasija and Norman, Genetics, 2015; Caldwell; Dis Model Mech; 2020). Reporter systems exist in C. elegans for sensing neuronal integrity (Martinez et al., J Neurosci, 2017), calcium signaling (Sarasija and Norman, Genetics, 2015)) and reduced lifespan (Mendenhall et al., J Gerentol A Biol Sci Med Sc, 2017), but are confounded by high background interference and a need for attenuation with specific genetic backgrounds. To circumvent these problems and enable easier automation of activity assessment, the methods disclosed herein can combine multiple genetic expression outputs to yield activity assays that are more amenable to high-throughput screening. In some such embodiments, split green fluorescent protein (GFP) can be used for precision tissue labeling (“split fluor technique”) as it reduces background signal. For instance, in some embodiments, a tissue specific promoter is used to express GFP1-10 in one or more particular tissue(s) for which the tissue specific promoter (TSP) drives gene expression. GFP1-10 is non-fluorescent due to a lack of the 11th beta sheet that is needed for hydrogen bond stabilization of the fluorescent chromophore. To create bimolecular fluorescence complementation, a GFP11 peptide can be co-expressed with GFP1-10 to enable a quenched chromophore environment and enable fluorescence emission upon exposure to the appropriate excitation wavelength. GFP1-10 protein can be expressed in the tissues in which the tissue-specific promoter is active (i.e., drives gene expression). A different or non- tissue-specific promoter can be used to drive GFP11 in a different subset of tissues (e.g., all tissues of the organism). In the tissues where expression of GFP1-10 and GFP11 overlap, co-expression of GFP1-10 and GFP11 can occur leading to fluorescent labeling of those tissues. Similar approaches can be carried out using other split GFP systems such as sfCherry. For instance, Phsp-6::sfCherry is a biomarker reporter construct indicative of the mitochondrial stress response and is expressed in target neurons as well as in the pharynx and gut. To achieve neuron-specific expression, Phsp-6::sfCherry1-10 (driving expression of GFP1-10 in dopaminergic neurons) can be combined with a Pdat-1::sfCherry11 (driving expression of GFP11), where overlapping activity the Phsp-6 and Pdat-1 promoters occurs exclusively in dopaminergic neurons such that both GFP1-10 and GFP11 are both expressed in those cells (FIG. 5A). Alternatively, in some embodiments, tissue-specific expression of a stress reporter can be targeted to all neurons via genetic cross of a C. elegans strain Phsp-6::sfCherry1-10 strain into a C. elegans strain harboring Psnb-1::sfCherry11. CRISPR-based gene editing can be used to introduce clinical variants of one or more ADRD gene(s) into either native or gene-humanized C. elegans loci. In one embodiment, the human sequence for human TARDBP human TARDBP can be inserted into the genome as a transgene at a safe-harbor locus and its expression was driven by a heterologous neuronal promoter (Psnb-1::hTARDBP::eft-3u). In another embodiment the human TARDB can be inserted into the native locus with the same sequence (Psnb-1::hTARDBP::eft-3u). In another embodiment, the TARDBP can be inserted into the native locus without the heterologous neuronal promoter and terminator sequence. In some embodiments, three clinically-observed pathogenic variants of TARDBP (G294A, G295S and/or G298S) can be installed in the humanized TARDBP locus. Functional testing on the wild-type and pathogenic variants preferably indicates a loss of cholinergic signaling in G294A and G295S but not G298S by assessment of paraquat hypersensitivity in a dye-filling assay of amphid neurons (FIG. 5B). As a complement, neuronal degradation can also be monitored via a thrashing assay to detect locomotion defects (Wang et al. PLoS Genetics, 2009). For drug screening, test compounds can be tested in these tissue-specific expression lines for their capacity to suppress the Phsp-6 stress reporter response and the resulting hits are validated by testing thrashing (phenotype) assays for restoration of normal activity. In a similar manner, in some embodiments, pathogenic variants of MAPT (e.g., G272V and P301L) in a humanized C. elegans MAPT line can be bred into the reporter background (i.e., C. elegans organisms expressing GFP11 in all tissues) and monitored for changes in stress reporter responses in the presence or absence of one or more test compounds.

In some embodiments (see, e.g., Example 10 herein), genetically encoded calcium indicators (GECls) can be used to uncover altered calcium signaling. For instance, in some embodiments, a Psnb-1 promoter is fused to a GCaMP7 (or similar GECI) to create a Psnb-1::GCaMP7 construct. The Psnb-1::GCaMP7 construct is integrated into the genome of C. elegans using CRISPR techniques. Fluorescence monitoring of the Psnb-1::GCaMP7 strain can be used to derive a baseline response for wild-type. The strain can then be genetically crossed into an appropriate C. elegans strain such as hTARDBP-wt and hTARDBP-G295S (or hTARDBP-G294A or hTARDBP-G298S) followed by monitoring for altered calcium signaling.

In some embodiments (see, e.g., Example 10 herein), a hTARDBP-wt can be incorporated into the zebrafish genome to replace the zebrafish homologs tardbp or tardbpl locus, and clinical variants (e.g., hTARDBP-G295S, hTARDBP-G294A, or hTARDBP-G298S) installed into this humanized locus (hTARDBP-wt). Concomitantly, a neuronal (e.g., elavl3) or glial (e.g., gfap) tissue-specific promoter can be operably linked to a GECI (e.g., GCaMP6) sequence and inserted into the zebrafish genome by either random integration (tol2 integrase) or safe-harbor site (phC31 integrase) to provide tissue-specific GECI reporters to produce zebrafish containing the transgene (elavl3:GCaMP6 or gfap:GCaMP6). The hTARDBP strains can be bred into the GECI reporter strains (or vica versa) which can then be monitored for altered calcium signaling due to defects in the TARDBP clinical variants. For drug screening, compounds can be tested in these tissue specific reporter lines for their capacity to restore normal calcium signaling (i.e., correct for the TARDBP clinical variant defect(s)). In some embodiments (see, e.g., Example 10 herein), MAPT variants (G272V and P301L) in a humanized MAPT line can be brought into the reporter background (strain expressing GFP11) and monitored for changes in calcium reporter responses in the presence or absence of the test compound(s). In some embodiments (see, e.g., Example 10 herein), shortened lifespan due to neurodegeneration can be monitored via automated lifespan analysis. A life-span/health-span instrument was adapted as a modified document scanner to enable repetitive imaging of a plate series throughout the lifespan of the transgenic C. elegans animal models. By plotting the series of images as a function of time to loss of activity for each animal can be determined. In some embodiments, techniques can be applied to images can uncover movement rates and behaviors that correlate with quality of health during lifespan (“healthspan”). Applied to both the C. elegans humanized hTARDBP and hMAPT strains described above, the behavioral activity throughout the life of the organisms can be used to profile differences in variant activity which then can be used as biomarkers for drug studies on finding compounds that reverse anomalous lifespan behavior back to normal.

In some embodiments (see, e.g., Example 10 herein), expression profiling using transcriptomics and proteomics can be used to detect shared mechanisms of dysfunction in gene variants involved in Alzheimer’s-related dementias. For instance, in some embodiments, RNAseq can be used to assay the transgenic TARDBP and MAPT C. elegans to create a profile of the changes in transcription and translation protein interactions. The profile changes can be mapped to nematode-customized ontologies that provide nodal foci of signaling pathway intersections. Disruption of signaling nodes in the nematode ontologies that have been cross referenced to human ontologies can provide an indication of mechanism of action in humans. Drug compound responses that lead to normal signaling strength in data flow through the ontologies. Restoration of normal ontology vector strength provides evidence for return to normal mechanistic function in the humanized variants harboring genetic lesions.

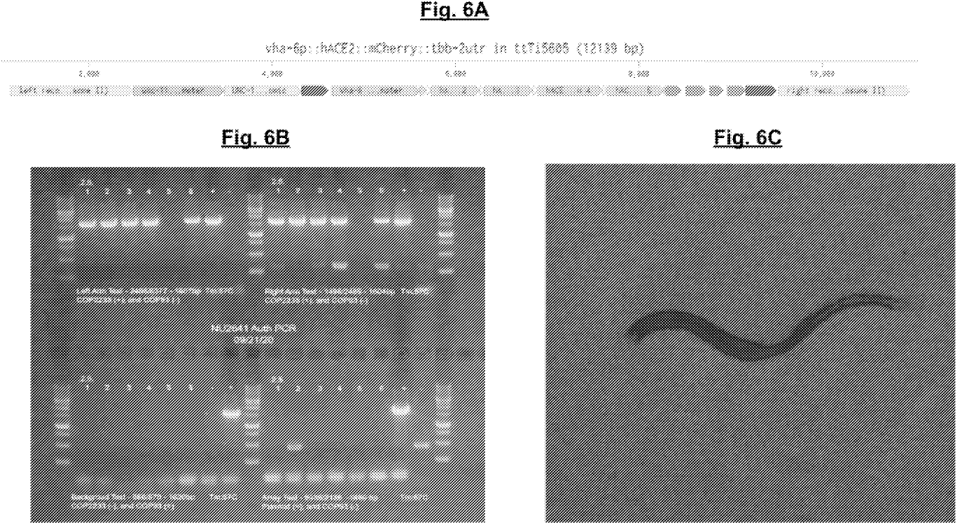

In some embodiments, this disclosure provides reagents and methods for studying infectious disease (see, e.g., Example 11 herein). In some such embodiments, reagents and methods for studying viral receptor engagement, viral entry into host tissues, and viral transcriptional responses in living organisms such as C. elegans and zebrafish are provided. In some embodiments, the human ACE2 receptor and the TMPRSS2 cofactor can be inserted into the C. elegans genome as a single copy, under the control of a tissue-specific promoters such as vha-6 for expression in the intestinal tissues using the MosSCI (Mos1-mediated Single Copy Insertion) method of transgenesis (Christian Frøkjaer-Jensen, et al., Single copy insertion of transgenes in C. elegans, Nat Genet. 2008 Nov; 40(11): 1375-1383. MosSCI transgenesis method can be used to insert genetic cargo at defined locations in the C. elegans genome using unc-119 rescue cassette insertion to bring genetic cargo (e.g., human homolog and/or clinical variant thereof) into a target locus (e.g., a C. elegans or zebrafish homologue of the clinical variant), thereby creating a rescue of function on the unc-119(ed3) III mutant allele. Targeting of cargo insertion occurs at select Mos1 loci. Each Mos1 locus used can be selected for position neutral effects and avoids the intragenic region of gene coding, introns, and transcription factor binding sites. The transgenic organisms (e.g., nematodes) created have the genotype [vha-6::TMPRSS2::GFP::tbb-2utr, unc-119(+))] II , unc-119(ed3) III] or [vha-6p::hACE2::mCherry::tbb-2utr, unc-119(+))] II , unc-119(ed3) III]. Validation using any suitable technique (e.g., PCR) can then performed to confirm the presence of the transgene in the organism (e.g., C. elegans or zebrafish), and fluorescent images confirming the protein-fluorophore fusion expression obtained (see, e.g., FIG. 6A). The transgenic strains produced as described herein can be used as created or crossed to produce transgenic strains expressing both the human viral receptors and any necessary co-factors for entry or replication. The resultant animals can be used in combination with exogenous biologic compounds such as live virus, pseudovirus, chimeric virus, viral fragments, and/or mRNA coding viral fragments to determine whether these exogenous biologics engage with the human viral receptors, and whether they are endocytosed and replicate in the C. elegans tissue. Because RNAi limits viral replication, the viral receptor and co-factor strains can be crossed with the rde-1 strain of C. elegans wherein RNAi machinery is defective to allow efficient replication. The resultant system can be used to determine whether candidate antigens interact with the viral receptors and elicit a cellular response. The system is a format in which the receptors, receptor variants and/or the antigens can be modified to provide a robust readout of receptor engagement and entry. The resultant system comprising animal models expressing viral-interacting proteins and biologics containing viral elements can then then used to screen for compounds that may inhibit viral engagement, endocytosis, replication. RNAseq can also be used to determine a transcriptional map of pro-viral and anti-viral response genes. Compounds can then be screened for anti-correlated transcriptional responses, indicating that compounds are effective in attenuating viral infection by increasing immune responses.

In some embodiments, ACE2-humanized transgenic zebrafish can be created as described herein for use as a high-throughput infection model for studying COVID-19 disease (see, e.g., Example 11 herein). In some embodiments, the method for transgene insertion can use a combination of CRISPR and phiC31 integrase activity. The phiC31 integrase technology can provide advantages because, unlike CRISPR based methods, the phiC31 integrase has a proven capacity to insert large segments of DNA content (~8kb) and at the high germline efficiency of up to 10% of an injected clutch of embryos. A two-step process can be used to create a humanized line. First, a germline knock-out can be made by introduction of an attP-stop in the first exon of ACE2 (the “attP-stop strain”). Next, the attP-stop strain can be used as substrate for knock-in insertion of human ACE2 cDNA (the “attP-stop ACE2 strain”). The germline knock-out (“attP-stop KO”) includes a small donor homology ODN brings in the 50 bp attP sequence and its inherent stop codon. Integration of this attP-stop site requires a pair of sgRNA/Cas9 nuclease sites arranged in a PAM-out configuration such that a 74 bp region is removed from the first exon of ace2. Cellular double-strand break repair machinery is used to repair the region with either Non-Homologous End Joining (NHEJ) or Homology-Directed Repair (HDR). Allele-specific PCR (ASPCR) can be used to detect if the desired HDR editing event has occurred. F0 animals with highest attP-stop signal in soma will be crossed with wild-type zebrafish. F1 embryos positive by the ZEG assay for attP-stop will be grown to adulthood and crossed again into wild-type to produce the F2 progeny (+/- heterozygote) that will be used in the construction of the humanized ACE2 strain. In some embodiments (e.g., ACE2 knock-in (“hACE2 KI”)), mRNA for phiC31-nanos1-3′UTR is used to provide transient expression of the integrase. Co-injection of the integrase with a plasmid containing an attB sequence (e.g., SEQ ID NO: 103)) can target gene insertion to occur with the reading frame of the gene remaining intact. A P2A self-cleaving peptide can be co-introduced between native and introduced cDNA sequence, which results in ACE2 cDNA being expressed with the addition of only one proline at the N terminus. ZEG (zebrafish embryo genotyper) assay with ASPCR can be used to find injected embryos with high levels of desired gene insertion. Animals with the highest ASPCR signals can be grown to adulthood and crossed into wild-type zebrafish. The resulting F1 cross progeny can be screened by ZEG and progeny positive for ACE2 gene insertion (+/- heterozygote) by ASPCR become founder animals for further studies (see, e.g., FIGS. 6A-6C).

In some embodiments, this disclosure provides nucleic acid constructs comprising a chimera of a C. elegans daf-12 DNA binding domain and a ligand binding domain of a nuclear hormone receptor operably linked to the coding sequence of a fluorescent reporter molecule, as well as C. elegans animals comprising one or more of such nucleic acid construct(s) incorporated into its genome. In some embodiments, this disclosure provides methods of screening human therapeutic agents that target one or more nuclear hormone receptors, the method comprising: treating such a transgenic C. elegans animal with a potential test compound; and, observing the phenotypic response of the transgenic C. elegans animal; wherein the phenotypic response indicates whether the test compound is an agonist or antagonist of the nuclear hormone receptor. In some embodiments, the nuclear hormone receptor is selected from the group consisting of VDR, RXRA, ESR1, PPARG, AHR, RARA, RARB, RARG, PPARA, PPARD, NR1D1, NR1D2, RORA, RORB, RORC, NR1H3, NR1H2, NR1H4, NR1H5P, NR1l2, NR1l3, HNF4A, HNF4G, RXRB, RXRG, NR2C1, NR2C2, NR2E1, NR2E3, NR2F1 ,NR2F2, NR2F6, ESR2, ESRRA, ESRRB, ESRRG, NR3C1, NR3C2, PGR, AR, NR4A1, NR4A2, NR4A3, NR5A1, NR5A2, NR6A1, NR0B1, and NR0B2.

In some embodiments, this disclosure provides nucleic acid constructs comprising a human GPCR gene operably linked to a native promoter of the human GPCR gene ortholog of a non-human animal, optionally wherein the human GPCR gene is HTR1A or HTR7, as well as animals comprising one or more of such nucleic acid constructs incorporated into its genome. In some embodiments, this disclosure provides methods of screening human therapeutic agents that target one or more G protein-coupled receptors (GPCR), the method comprising: inserting a nucleic acid construct of any one of claims 19-21 into the genome of the non-human animal; randomly mutagenizing the native promoter to create downward attenuated expression of the human GPCR gene and a corresponding loss-of-function (LOF) phenotype in the non-human animal; and, treating the non-human animal with one or more GPCR agonists to identify a therapeutic agent that restores the LOF phenotype.

In some embodiments, this disclosure provides nucleic acid constructs comprising a promoter regulatory sequence for ubiquitous expression in C. elegans neurons or in zebrafish, a human cannabinoid receptor optimized for expression in C. elegans, and a 3′ untranslated region (UTR), optionally wherein the human cannabinoid receptor is selected from the group consisting of CNR1, SEQ ID NO: 12, CNR2, and SEQ ID NO: 14, as well as a C. elegans animal or zebrafish comprising the nucleic acid construct of claim 23 or 24 incorporated into its genome. In some embodiments, the C. elegans animal or zebrafish does not express at least one native endocannabinoid synthesis gene product, is depleted in or over-expresses the fatty acid amide hydrolase (faah-2) or ortholog thereof, and/or expresses at least one human clinical variant. In some embodiments, this disclosure provides methods of identifying a therapeutic agent for cannabinoid treatment by exposing such a C. elegans animal or zebrafish to a potential therapeutic agent and detecting phenotypic changes in the C. elegans animal or zebrafish.

In some embodiments, this disclosure provides a C. elegans animal comprising a nucleic acid sequence encoding the coding sequence for human gamma-aminobutyric acid transaminase (hGABAT) in its genome, optionally wherein the nucleic acid sequence is SEQ ID NO: 16 or encodes a gene provided in Table 6. In some embodiments, the C. elegans animal further comprises within its genome at least one additional nucleic acid sequence encoding at least one additional human gene, optionally wherein the at least one additional human gene is hSTXBP1. In some embodiments, this disclosure provides methods of identifying a therapeutic agent for cannabinoid treatment by exposing such a C. elegans animal to a potential therapeutic agent and detecting phenotypic changes in the C. elegans animal.

In some embodiments, this disclosure provides a C. elegans animal or zebrafish comprising a nucleic acid sequence encoding the coding sequence for a genetic variant associated with Alzheimer’s Disease in its genome, optionally wherein the nucleic acid sequence comprises a coding sequence of a gene selected from the group consisting of MAPT, GRN, TARDBP, APP, PSEN1, and PSEN2. In some such embodiments, the nucleic acid sequence is operably linked to at least one reporter sequence, optionally wherein the reporter is operably linked to a tissue-specific promoter that in some embodiments is specific for neurons. In some embodiments, the reporter sequence is a fluorescent protein or a genetically encoded calcium indicators (GECI). In some embodiments, this disclosure provides methods for identifying a therapeutic agent for Alzheimer’s Disease, the method comprising exposing such a C. elegans animal or zebrafish to a potential therapeutic agent for Alzheimer’s Disease and detecting phenotypic changes in the C. elegans animal or zebrafish.

In some embodiments, this disclosure provides a C. elegans animal or zebrafish comprising a nucleic acid sequence encoding a human viral receptor in its genome, optionally wherein the nucleic acid sequence comprises a coding sequence of ACE2, optionally wherein the nucleic acid is optimized for expression therein. In some such embodiments, the nucleic acid sequence is operably linked to at least one reporter sequence, optionally wherein the reporter is a fluorescent reporter. In some embodiments, this disclosure provides methods of identifying a therapeutic agent for an infectious disease, the method comprising exposing such a C. elegans animal or zebrafish to a potential therapeutic agent for the infectious disease and detecting phenotypic changes in the C. elegans animal or zebrafish.

Other embodiments are also contemplated herein as will be understood by those of ordinary skill in the art from this disclosure.

All references cited within this disclosure are hereby incorporated by reference in their entirety. Certain embodiments are further described in the following examples. These embodiments are provided as examples only and are not intended to limit the scope of the claims in any way.

### EXAMPLES

The following examples are put forth so as to provide those of ordinary skill in the art with a complete disclosure and description of how to use the embodiments provided herein and are not intended to limit the scope of the disclosure nor are they intended to represent that the Examples below are all of the experiments or the only experiments performed. Efforts have been made to ensure accuracy with respect to numbers used (e.g. amounts, temperature, etc.) but some experimental errors and deviations should be accounted for. Unless indicated otherwise, parts are parts by volume, and temperature is in degrees Centigrade. It should be understood that variations in the methods as described can be made without changing the fundamental aspects that the Examples are meant to illustrate.

### Example 1: Transgenic C. Elegans Lines for SLC6A4-Association Conditions

Provided herein are transgenic nematodes and methods of preparing same for expression, as well as methods for using the same drug discovery for human therapeutic targets, such as that illustrated in FIG. 1 and described in more detail herein, wherein the therapeutic target gene is first optimized for expression in a host nematode and then incorporated into the genome of the nematode. Human Solute Carrier Family 6 Member 4 (SLC6A4) is known to transport serotonin out of the synaptic space, thus terminating the action of serotonin. By way of example to demonstrate the general principle of expressing therapeutic targets in the nematode (C. elegans), the human coding sequence SLC6A4 was inserted into the C. elegans genome. Inhibitors of SLC6A4, selective serotonin reuptake inhibitors (SSRls), are commonly used to treat major depressive disorder and anxiety but are also known to cause many unpleasant side-effects. New SSRls can be discovered using the SLC6A4 overexpressing C. elegans transgenic nematodes described in this illustrative example. As shown below, C. elegans transgenic animals overexpressing human SLC6A4 in the pharyngeal muscle show a decrease in pumping frequency when stimulated to pump by food (a mild stimulus) as compared to the wildtype. These animals can be screened to identify novel SSRls. See FIG. 2C. For instance, on the first day adult C. elegans transgenic animals can be soaked in drug and food stimulus for 30 minutes, and then assayed for pumping frequency using the ScreenChip™ System. Each EPG recording is two minutes in duration and the experiment is replicated in triplicate on different days. An increase in pumping frequency in the SLC6A4 transgenic animals indicates that the drug is acting as an inhibitor to serotonin uptake.

The human coding sequence SLC6A4 was optimized for expression in C. elegans. Artificial C. elegans introns added and aberrant splice sites removed, and then inserted into the C. elegans genome to produce transgenic SLC6A4 C. elegans animals. The modified SLC6A4 sequence, including artificial nematode introns as lower-case letters (and shown below as SEQ ID NOS. 2-4), is shown below:

ATGgctagcAGCGCTGAGCTACATCAAGGGGAACGTGAAACATGGGGAAA
GAAAGTCGACTTCCTCCTCTCCGTCATCGGATACGCCGTCGACCTCGGAA
ACGTCTGGCGTTTCCCATACATCTGCTACCAAAACGGAGGAGGAGCCTTC
CTCCTCCCATACACCATCATGGCCATCTTCGGAGGAATCCCACTCTTCTA
CATGGAGCTCGCCCTCGGACAATACCACCGTAACGGATGCATCTCCATCT
GGCGTAAGATCTGCCCAATCTTCAAGGGAATCGGATACGCCATCTGCATC
ATCGCCTTCTACATCGCCTCTTACTACAACACCATCATGGCCTGGGCCCT
CTACTACCTCATCTCCTCCTTCACCGACCAACTCCCATGGACCTCCTGCA
AGgtacttgagatccttaaacgcagtcgaaaattggtaattttacagAAC
TCCTGGAACACCGGAAACTGCACCAACTACTTCTCCGAGGACAACATCAC
CTGGACCCTCCACTCCACCTCCCCAGCCGAGGAGTTCTACACCCGTCACG
TCCTCCAAATCCACCGTTCCAAGGGACTCCAAGACCTCGGAGGAATCTCC
TGGCAACTCGCCCTCTGCATCATGCTCATCTTCACCGTCATCTACTTCTC
CATCTGGAAGGGAGTCAAGACCTCCGGAAAGGTCGTCTGGGTCACCGCCA
CCTTCCCATACATCATCCTCTCCGTCCTCCTCGTCCGTGGAGCCACCCTC
CCAGGAGCCTGGCGTGGAGTCCTCTTCTACCTCAAGCCAAACTGGCAAAA
GCTCCTCGAGACCGGAGTCTGGATCGACGCCGCCGCCCAAACTTCTTCTC
CCTCGGACCAGGATTCGGAGTCCTCCTCGCCTTCGCCTCCTACAACAAGT
TCAACAACAACTGCTACCAAGACGCCCTCGTCACCTCCGTCGTCAACTGC
ATGACCTCCTTCGTCTCCGGATTCGTCATCTTCACCGTCCTCGGATACAT
GGCCGAGATGCGTAACGAGGACGTCTCCGAGGTCGCCAAGgtaagttcct
ccactagaaatatcaggtgctataattgtgttcagGACGCCGGACCATCC
CTCCTCTTCATCACCTACGCCGAGGCCATCGCCAACATGCCAGCCTCCAC
CTTTTTTGCCATCATTTTCTTCCTCATGCTTATAACCCTCGGACTCGACT
CCACCTTCGCCGGACTCGAGGGAGTCATCACTGCCGTCCTCGACGAGTTC
CCACACGTCTGGGCCAAGCGTCGTGAGCGTTTCGTCCTCGCCGTCGTCAT
CACCTGCTTCTTCGGATCCCTCGTCACCCTCACCTTCGGAGGAGCCTACG
TCGTCAAGCTCCTCGAGGAGTACGCCACCGGACCAGCCGTCCTCACCGTC
GCCCTCATCGAGGCCGTCGCCGTCTCCTGGTTCTACGGAATCACCCAATT
CTGCCGTGACGTCAAGgtgagttattataatttttttgatcacaacgatt
attttaattttcagGAGATGCTCGGATTCTCCCCAGGATGGTTCTGGCGT
ATCTGCTGGGTCGCCATCTCCCCACTCTTCCTCCTCTTCATCATCTGCTC
CTTCCTCATGTCCCCACCACAACTCCGTCTCTTCCAATACAACTACCCAT
ACTGGTCCATCATCCTCGGATACTGCATCGGAACCTCCTCCTTCATCTGC
ATCCCAACCTACATCGCCTACCGTCTCATCATCACCCCAGGAACCTTCAA
GGAGCGTATCATCAAGTCCATCACCCCAGAGACCCCAACCGAGATCCCAT
GCGGAGACATCCGTCTCAACGCCGTCTAA (SEQ ID NO: 1).

The artificial introns were designed based on small introns in highly expressed native C. elegans genes. The intron sequences maintain the coding frame and, if they were translated, the amino acid sequence would not contain stop codons and would have a low hydropathy index. In the SLC6A4 optimized construct (SEQ ID NO: 1), the sequences of the artificial introns are shown below:

1) gtacttgagatccttaaacgcagtcgaaaattggtaattttacag (
SEQ ID NO: 2);

2) gtaagttcctccactagaaatatcaggtgctataattgtgttcag (
SEQ ID NO: 3); and,

3) gtgagttattataatttttttgatcacaacgattattttaattttca
g (SEQ ID NO: 4).

The optimized SLC6A4 sequence was obtained as a gene block from IDTDNA, Inc, and cloned into a donor homology plasmid (pNU1313) using standard techniques (Gibson et al. Nat. Methods, 6(5):343-5 (2009)). The donor homology plasmid pNU1313 also contained the C. elegans myo-2 promoter to induce expression of the SLC6A4 in the pharynx muscle cells. Additionally, the tbb-2 3′UTR was added to complete the expression system. The plasmid backbone also contained the homology arms for insertion into the ttTi14024 Mos1 insertion site and the unc-119 rescue cassette (Frokjaer-Jensen et al. Nat Methods. 9(2):117-8 (2012)). After Gibson assembly, the plasmid sequence was confirmed by sequencing. A transgenesis mixture containing pNU1309 along with standard MosSCI injection components was injected into gonads of the host C. elegans strain EG6705 using standard microinjection techniques (Frøkjaer-Jensen, et al. Nat Genet. 40(11):1375-83 (2008); Evans TC. Transformation and microinjection (Apr. 6, 2006). In (The C. elegans Research Community, ed) WormBook. doi/10.1895/wormbook.1.108.1). Crawling animals that lacked the red fluorescent array markers and that survived heatshock were confirmed by PCR to contain the insertion at the ttTi14024 locus.

The SLC6A4 transgenic C. elegans line was designed to give high levels of gene expression in the pharynx and lead to abundant levels of SLC6A4 at the plasma membrane of the pharynx muscle cells in order to decrease pumping frequency in the presence of mild pumping stimulants. The ScreenChip™ (NemaMetrix, Inc.) was used to collect electrophysiological data for pharyngeal pump frequency, pump duration and pump interval. First-day adults were incubated in OP50 E. coli food or 10mM serotonin for 20 minutes prior to commencing EPG recordings. The expression of SLC6A4 at the pharynx causes a pronounced defect in pharynx pumping rate in the presence of mild stimulation with food (FIG. 2A). The WT pumping frequency of 2.26 Hz (n=10) dropped to a rate of 0.03 Hz (n=6) for pharynx::SLC6A4. The most likely cause for the lower pumping rate in the SLC6A4 line is that the overexpression of exogenous transporter removes trace levels of serotonin from the synaptic cleft which in turn renders pumping stimulation to remain at low activation after exposure to food. Only when exogenous serotonin was added to the media at high concentration did the rate of pumping become restored (FIG. 2B). The level of restoration is quite significant — starting from a 50-fold difference with food stimulation, the WT and SLC6A4 construct become indistinguishable after 10 mM serotonin (also referred to as “5HT” or 5-Hydroxytryptamine) exposure. These transgenic C. elegans animals overexpressing SLC6A4 can be used for SSRI discovery. See FIG. 2C.

In some embodiments, and by way of example to demonstrate the general principle of modeling common genetic variants for pharmacogenetic studies, the humanized SLC6A4 C. elegans transgenic animals described above is injected with CRISPR/Cas9 components to create animals expressing SLC6A4 variants. Two common SLC6A4 variants, Gly56Ala and Lys605Asn, are each incorporated into the genome of a cell and/or animal using CRISPR/Cas9 for precise genome editing. Antagonists are also screened using these variant containing animals and the effect size is measured. As described above, the Gly56Ala and Lys605Asn lines are treated with compound and pharyngeal pumping is tested with the ScreenChip™ to associate a phenotypic change with that compound.

### Example 2. Transgenic C. Elegans for KCNQ2-Association Conditions

KCNQ2 is an important human disease-associated gene associated with, for example, early infantile epileptic encephalopathy 7; Myokymia; and benign neonatal seizures. The retigabine agonist of KCNQ2 is used to treat loss-of-function (LOF) variant activity in epilepsy patients (Gunthorpe et al. Epilepsia. 53(3):412-24 (2012)). Conversely, gain-of-function (GOF) in KCNQ2 is also associated with epilepsy (Niday and Tzingounis. Neuroscientist. 24(4):368-380 (2018)). As a result, either an agonist or an antagonist of KCNQ2 are needed for treatment of KCNQ2-associated epilepsies. The KCNQ2 gene was gene-swapped into the kqt-1 locus of C. elegans animals, but deviations in electrical activity were absent in gene knock-out of the locus when measured by electropharyngialgram (EPG). Consistent with published RNA-seq data, the absence of phenotype in transgenic adult stage C. elegans is best explained by lack of gene expression. To generate functional consequence in adult C. elegans animals, ectopic overexpression was deployed. A KCNQ2 sequence optimized for expression in C. elegans was designed and is shown below as SEQ ID NO: 5:

ATGCTAACAACCGGTGGATCGGGTGGATCGATGGTACAAAAGTCCAGAAA
TGGTGGAGTTTACCCGGGTCCATCTGGTGAAAAAAAATTGAAAGTAGGAT
TTGTCGGCCTCGACCCTGGAGCGCCGGACAGTACCAGAGATGGCGCGCTG
TTGATCGCTGGTTCGGAGGCACCGAAACGAGGAAGTATTCTCAGTAAGCC
TCGTGCGGGAGGTGCCGGCGCTGGAAAACCGCCTAAAAGAAATGCCTTTT
ACAGAAAGCTGCAGAACTTCTTGTATAATGTGCTGGAACGACCGAGAGGC
TGGGCATTTATTTATCACGCCTACGTTTTCTTGCTTGTTTTCTCCTGCCT
TGTGTTGAGTGTTTTCTCCACCATAAAAGAATACGAAAAAAGTTCCGAGG
GTGCTCTTTACATCCTCGAAATTGTCACCATCGTGGTGTTCGGAGTGGAA
TACTTTGTTAGAATTTGGGCCGCTGGCTGCTGCTGCCGATACCGAGGCTG
GCGAGGTCGTCTGAAATTTGCTCGAAAACCGTTCTGTGTCATCGACATTA
TGGTTCTGATCGCAAGTATTGCTGTCTTGGCGGCGGGATCTCAGGGCAAT
GTGTTTGCAACCTCGGCCCTTAGATCCCTCCGATTTTTACAAATCCTCCG
TATGATCCGTATGGACCGACGTGGTGGAACTTGGAAACTTCTTGGATCCG
TCGTCTACGCCCACTCCAAGgtgagtgattttaaacattatctgtactta
aattataaattctctattcagGAACTCGTCACCGCCTGGTACATCGGATT
CTTGTGTCTTATCCTGGCATCGTTTCTTGTTTACTTGGCCGAAAAGGGTG
AAAACGATCACTTTGACACATATGCCGATGCGTTGTGGTGGGGCTTGATC
ACTCTTACGACAATTGGATATGGTGACAAGTATCCGCAGACATGGAATGG
TAGACTTCTTGCTGCCACCTTCACCCTGATCGGTGTCAGTTTCTTCGCCC
TCCCAGCTGGCATCCTGGGCTCAGGTTTTGCGCTGAAGGTCCAAGAGCAG
CACCGACAAAAACACTTTGAAAAGCGACGTAACCCTGCCGCTGGTTTGAT
TCAATCCGCTTGGAGATTCTACGCTACGAACTTGTCTCGTACCGATCTGC
ACTCTACCTGGCAATACTACGAAAGAACGGTAACAGTGCCGATGTATTCG
TCCCAAACTCAAACTTACGGAGCTTCAAGACTGATTCCACCGCTGAACCA
GCTGGAGCTGTTGCGAAACCTTAAATCAAAATCTGGCCTGGCTTTCCGAA
AGGATCCTCCTCCGGAGCCTTCGCCTTCTAAGGGAAGTCCTTGCAGAGGC
CCGCTTTGCGGTTGCTGCCCAGGACGTTCCTCCCAAAAGgtaaataatta
tacattcgatgataaatttatgcgtactatttttcagGTCTCCCTCAAGG
ACCGTGTCTTCTCCTCCCCGAGAGGCGTAGCAGCCAAGGGAAAGGGAAGT
CCACAAGCACAAACTGTTCGAAGATCGCCTTCAGCGGACCAATCATTGGA
AGACTCGCCATCAAAGGTGCCTAAATCCTGGTCCTTTGGTGACCGTTCGA
GAGCAAGACAGGCCTTCCGTATCAAGGGTGCGGCATCTCGACAGAATTCG
GAAGAAGCTTCACTCCCAGGCGAGGACATCGTGGACGACAAATCTTGTCC
GTGTGAATTTGTGACCGAAGACCTCACTCCGGGTTTGAAAGTGTCTATCA
GAGCGGTGTGCGTGATGAGATTCCTCGTCTCCAAGCGTAAATTCAAGGAA
TCCTTGCGACCGTATGACGTTATGGACGTTATCGAACAATACTCAGCTGG
ACATTTGGATATGCTTTCGCGTATCAAGTCCCTCCAAAGTAGAGTGGACC
AAATTGTTGGCAGAGGACCTGCAATCACCGACAAGGACAGAACGAAGGGT
CCTGCGGAAGCCGAGCTGCCTGAGGACCCATCAATGATGGGTAGATTGGG
CAAGGTTGAAAAACAAGTTTTGAGTATGGAGAAGAAACTGGACTTTCTTG
TCAATATCTATATGCAAAGAATGGGAATCCCTCCTACGGAGACCGAGGCC
TACTTCGGAGCCAAGgttaaatgtacaaacaactatttgaaagattttct
cacccgattttttcagGAGCCCGAGCCAGCCCCTCCATACCACTCACCAG
AAGACTCACGTGAACACGTTGACAGACACGGTTGCATTGTGAAAATTGTT
CGTTCTTCGTCCTCGACGGGTCAGAAAAACTTCTCAGCACCACCTGCTGC
CCCTCCTGTCCAATGCCCTCCGTCAACTAGTTGGCAACCGCAAAGTCATC
CGCGTCAGGGCCATGGTACGAGTCCAGTAGGCGATCACGGCTCGTTGGTG
CGAATCCCGCCTCCTCCTGCCCACGAGAGATCATTGTCTGCCTACGGTGG
CGGCAATCGAGCATCTATGGAGTTCCTGAGACAAGAAGACACCCCAGGAT
GCAGACCGCCAGAGGGTAACCTTCGTGACTCTGACACGTCCATTTCAATC
CCTTCAGTTGACCACGAAGAACTCGAGAGATCCTTCAGTGGATTTTCCAT
CTCTCAATCTAAAGAAAATCTGGATGCCCTCAACTCATGTTATGCGGCGG
TCGCACCGTGTGCAAAGGTTCGTCCTTACATCGCGGAGGGAGAGAGTGAC
ACAGACAGTGACCTGTGCACGCCTTGCGGACCGCCGCCACGATCAGCTAC
CGGAGAAGGCCCTTTCGGTGATGTGGGATGGGCAGGCCCTCGAAAATAA
(SEQ ID NO: 5).

In some embodiments, transgenic KCNQ2 C. elegans animals can be used for the detection of general antagonist activity with tissue-specific overexpression. In this embodiment, a snb-1 pan-neuronal promoter sequence is inserted upstream of the humanized KCNQ2 gene start codon resulting in KCNQ2 overexpression in all neuronal cells. The result is a greater sensitivity to transgene activity. The snb-1 promoter region (SEQ ID NO: 7) is inserted at the TTTTGCATCCGAAAAAGCGG (SEQ ID NO: 6) sgRNA site, which occurs adjacent and upstream of the humanized (KCNQ2) coding sequence. The result is a snb-1-enhanced KCNQ2 animal model. The snb-1 promoter region is shown below:

cttatcatttcaatttatttattaatcgatgattgaaagtgaatggatga
cggtcatgaccgattatcgattatcccgaaatagagatgcgcgtaggtca
taatgcccagtacgcaaaatgttttatcggtgtttgcacagatttcgcaa
catctctcattgaatttccattcatcgcttcgtcatctgaccccatttct
tattttttcatccttttccctgttctcatcgttccttactattttcctaa
tttcagaacatcgcgattttataatttcgttaaatattcgtaatcccgtt
atacaaaaatagctaaattttctagtcgttctcgtttttgagagggcact
ttagtccgtcatcgtgtcgcttgtcgtgctcaatttttcatgcataaatg
ggcgtcgccgtcccccctgtcgttttcttcctttacctcactttccagtt
ctgaattccgatacgaatttttaaatttttctaactcgcttcatttcagg
g (SEQ ID NO: 7).

The insertion of snb-1 promoter before the human coding sequence leads to hypermorphic over-expression of the human transgene in C. elegans. The increased activity level leads to severe attenuation of electrical signals due to excessive M-current activity. The result is an animal with severely inhibited (suppressed) pharyngeal pumping activity. Exposure of an antagonist to the animal leads to restoration of near wild type activity. As a result, the snb-1-enhanced KCNQ2 transgenic C. elegans animal can serve as a screening tool for finding KCNQ2 antagonists.

In one embodiment, the snb-1-enhanced KCNQ2 line described above is modified to contain a clinical variant known to cause loss of function (LOF). CRISPR-mediated gene editing is used to install a LOF pathogenic allele (clinical variant). Expression of the LOF variant in the humanized locus creates an animal exhibiting a return towards normal pumping rates. The addition of agonist will lead to a return to suppressed pumping rate. As a result, the system with a LOF variant incorporated into the genome of a cell and/or animal can be used for highly personalized discovery of patient-specific agonist therapeutics.

In one embodiment, the KCNQ2 humanized line is enhanced with a cell-specific ceh-2 promoter. The ceh-2 gene is primarily expressed in the M3 inhibitory neuron, whose activity is necessary for attenuating pharyngeal pump rates (Routhan et al. Dev Biol. 2007 Nov 1;311(1):185-99). To make a ceh-2-enhanced line, the ceh-2 promoter is inserted into the TTTTGCATCCGAAAAAGCGG (SEQ ID NO: 8) sgRNA site. The cell-specific expression of KCNQ2 occurs only in the inhibitory M3 neurons. The overexpression of KCNQ2 is excessive M-current activity, which will cause lower activity from the inhibitory neuron and lead to greater than normal (hyperactive) pumping rate will occur. The effect is M3 neurotransmission is severely attenuated and hyperactive pharyngeal pumping is the result. The detection of hyperactive pumping is detected by EPG. Exposure to inhibitors of potassium channels leads restoration of normal EPG rates. As a result, the ceh-1-enhanced KCNQ2 line has become a screening tool for finding antagonist of KCNQ2. The promoter region for ceh-2 gene (SEQ ID NO: 9) is shown below:

cgattactaacataatatacctatttctgagcccaacgtccgctgctttc
gggcgcacaaaagatgtaatccgagatctgcgatttgtacatatttcgga
tacattttcaatctccatttccaactcatcttctaattcgggacaatgag
catcatcatgagcatgtgtacacaaggtagttcggtcgcccagttgccga
ttacataggcacccggttgccgagtagacgagttgtaaaaacagggcacc
acgagatattgccgatttccacataaactacttattaaagtctggagatt
taattcatatgtcaagaaaaatgttcgtgaaagcaacaatatatgtacta
gaaatccctaagacgacgactatcgctgacgttacattgctccaaaagaa
tgcaatgaattgtgattacaccatgagagttttcaaaactttaaacctca
aattaatatttcaacttattaacctacttcctattcttttcaattcttta
tttgtctatcgtctctttattcccaatgatctctcttctaagagcttcta
ccttatcggctactgtgtccaattacccatcgacgctcctccctcttttg
cgcgaaactgggcggagcctaagatgagattcagtaaaataggttctc
(SEQ ID NO.: 9)

In one embodiment, specific agonist activity is detected for a clinical variant causing gain of function (GOF) activity. A GOF pathogenic allele (clinical variant) that is known to cause epilepsy is incorporated into the genome of a cell and/or animal into the ceh-2-enhanced KCNQ2 line. The cell-specific expression of KCNQ2 GOF variant in the M3 neuron leads to a return towards normal pumping rates, because only partial and insufficient activity is expected from the clinical variant. When exposed to an antagonist, the animal activity returns to hyperactive pumping rates. As a result, the system with a GOF variant incorporated into the genome of a cell and/or animal can be used for highly personalized discovery of patient-specific antagonist therapeutics.

### Example 3: DAF-12 Transgenic C. Elegans (daf-12); Use of C. Elegans Reporter Lines for Agonist and Antagonist Discovery in Nuclear Hormone Receptors

To create a platform specific to human NHR signaling pathway activation, a chimera is made by fusing the DNA binding domain of daf-12 to the ligand binding domain of various drug and cytotoxic agents. Nuclear hormone receptors are common targets for drug discovery as their function can often be modulated by small molecules, accounting for the therapeutic effect of 16% of all small-molecule drugs (Santos et al. Nature reviews: Drug discovery, Jan; 16(1): 19-34 (2017)). Both antagonist and agonist ligands of NHRs are involved in treating cancer (Safe et al. Mol Endocrinol. 2014 Feb;28(2):157-72) and environmental toxicity (Ren and Gao. Environ Sci Process Impacts. 2013 Apr;15(4):702-8).

Important targets for creation of DAF-12 chimeras can include NHRs involved in hormonal signaling (e.g., VDR, RXRA, ESR1, PPARG) and genes involved in biosensing environmental chemicals (e.g., AHR). Other NHR type for drug and toxicity discovery are RARA, RARB, RARG, PPARA, PPARD, NR1D1, NR1D2, RORA, RORB, RORC, NR1H3, NR1H2, NR1H4, NR1H5P, NR1l2, NR1l3, HNF4A, HNF4G, RXRB, RXRG, NR2C1, NR2C2, NR2E1, NR2E3, NR2F1 ,NR2F2, NR2F6, ESR2, ESRRA, ESRRB, ESRRG, NR3C1, NR3C2, PGR, AR, NR4A1, NR4A2, NR4A3, NR5A1, NR5A2, NR6A1, NR0B1, and NR0B2.

Chimeric proteins made with the daf-12 nuclear hormone receptor (NHR) can be used for discovery of agonist and antagonist of human NHRs and detect ligands of human NHRs. In C. elegans, the daf-12 protein activity is a key regulator of development. When daf-12 is activated by dafachronic acid under normal growth conditions, animals will proceed to their reproductive life stage. However, when animals face unfavorable conditions, dafachronic acid is not produced and the dauer program is activated leading animals into a stress-resistant arrested state. The unfavorable conditions can be mimicked by treatment with a DAF-12 antagonist, dafadine. Dafadine treatment overcomes the growth condition signal and animals enter the arrested state. Reporter lines can be used to monitor daf-12 chimera activity. In order to create reporter lines for agonist and antagonist discovery, the transcriptional response to treatment with dafachronic acid or dafadine was determined. Dauer animals were treated with 500 nM dafachronic acid and harvested for RNA preparation after four hours of exposure. RNA samples were processed and sent for RNA sequence analysis. Alternatively, in some embodiments, qPCR of predicted targets can be performed. Candidates for agonist reporters are genes that are strongly upregulated in the treated animals versus were non-treated controls.

In this illustrative example, animals in the first larval stage were treated with 100uM dafadine for four hours and harvested for RNA preparation and analysis. Two reporter lines were identified by observation of fluorescence after drug treatment. A photograph of C. elegans lin-42::GFP, after dafachronic acid exposure, ranging in concentration from 0 µM to 12 µM, provides a correlation between the role of dafachronic acid as an agonist of the chimeric lin-42::GFP promoter fusion (FIG. 3A). A lin-42::GFP reporter as a promoter fusion showed induced fluorescence with dafachronic acid exposure and reported agonist activity of compounds. The lin-42::GFP reporter was titrated with dafachronic acid for its agonist response and an EC50 near 0.49 nM was obtained (FIG. 3D). The animal’s phenotypic response to dafachronic acid was measured and an EC50 of 17nM was obtained (FIG. 3E). The agonist response of lin-42::GFP to dafachronic acid was more sensitive than the phenotype and data indicates that the reporter is at least 40x more sensitive than the phenotypic assay. An mtl-1::GFP reporter as a promoter fusion showed induced fluorescence with dafadine exposure and detection antagonist activity. The mtl-1::GFP reporter gave titratable response to dafadine (FIG. 3A) and gave an EC50 of 5.2 (FIG. 3C). The phenotypic assay gave an EC50 of 48 uM. The reporter gave a response that was 9x more sensitive than the phenotypic assay. These results show the fluorescence reporter is more favorable for drug screening because 10x less compound is needed to enable detection of response.

### Example 4: GPCR Transgenic C. Elegans (HTR1A, HTR7)

To create a platform system for detecting GPCR agonists, the human gene is first inserted into the genome under its gene ortholog’s native promoter. When the gene is observed as capable of rescuing function, the promoter is “bashed” by random mutagenesis to create downward attenuated expression. For instance, existing ChlP-seq data is used to determine promoter binding elements region upstream of transgene start codon. CRISPR-mediated donor homology insertion with ODN templating is used to alter codon composition in the promoter. Alternatively, non-templated CRISPR-mediated error-prone repair can be used. When a promoter-bashed animal starts to exhibit a phenotype similar to the KO allele, a drop in expression is verified by rtPCR. When an animal is found exhibiting a loss-of-function (LOF) phenotype and a significant drop in transgene expression, the promoter-bashed system becomes a platform for discovery of agonists that restore behavior back to normal activity. Tables 3 and 4 above describe exemplary GPCR genes and their corresponding C. elegans orthologs.

By way of demonstration in C. elegans, the HTR1A gene is inserted as gene-replacement of the coding sequence of orthologous ser-4. The HTR1A gene is associated with mood disorders (Garcia-Garcia et al. Psychopharmacology (Berl). 2014 Feb;231(4):623-36) and periodic fever that is menstrual cycle dependent. Rescue of movement defects is used to confirm the human HTR1A transgene functions in a similar manner as the ser-4 ortholog. The ser-4 promoter is bashed via CRISPR-based methods to randomly introduce changes that decrease hHTR1A expression. Concomitant LOF is observed and movement defects return to the animal. rtPCR is used to confirm transgene expression has dropped by 10x. As positive control, the strain is exposed to a set of known FDA-approved agonists (cinitapride, ergoloid, flibanserin, lisuride, naratriptan, sumatriptan, vilazodone, vortioxetine, and zolmitriptan) to see by how much restoration of normal behavior can be achieved. The EC50 for each drug is determined for capacity to restore behavior. As negative control, the strain is exposed to FDA-approved antagonist (acepromazine, alverine, asenapine, clozapine, iloperidone, lurasidone, methysergide, olanzapine, pipotiazine, thioproperazine and trazodone) and inability to rescue function is measured. Next, novel compounds in clinical trials for agonists are exposed to the promoter bashed strain (eltoprazine, mn-305, opc-14523, pardoprunox, piclozotan, prx-00023, psn602, SEP-363856, sumatriptan, vibegron, xaliproden, and zolmitriptan) at the average EC50 concentration of known agonists. Good candidates for drug development are revealed when candidate compounds are observed to cause restored behavior of the animal similar to the native-expressed hHTR1A strain.

To create a GPCR antagonist detection system, the human gene is first inserted into the genome under its gene ortholog’s native promoter. When the gene is observed as capable of rescuing function, heterologous promoter insertions are used to upward attenuate expression. In C. elegans for instance, the eft-3 promoter is inserted to create higher expression of human transgene in all tissues. When this is too toxic to the animal, alternative promoters such as pan-neuronal snb-1 are used to get tissue-specific overexpression. When an overexpressed transgene system is observed to exhibit deviant behavior, the increase in expression is verified by rtPCR. When an animal is found exhibiting a gain of function phenotype and a significant increase in transgene expression, the promoter-bashed system becomes a platform for discovery of antagonists that restore behavior back to normal activity.

By way of demonstration in C. elegans, the HTR7 gene is inserted as gene-replacement of the coding sequence of orthologous ser-7. The HTR7 gene is linked to depression (Hedlund. Psychopharmacology (Berl). 2009 Oct;206(3):345-54). The ser-7 promoter primarily expresses in pharyngeal neurons (Hobson et al. Genetics. 2006 Jan;172(1):159-69). Rescue of pharyngeal movement defects is used to confirm the human HTR7 transgene functions in a similar manner as the ser-7 ortholog. Next, the highly-expressing, pan-neuronal promoter, snb-1p, is inserted upstream of the ser-7 start codon to induce hypermorphic expression in neuronal tissues. Concomitant GOF is observed because movement defects from transgene overexpression occur in the animal. rtPCR is used to confirm transgene expression has increased by close to 10x. As positive control, the strain is exposed to a set of known FDA-approved antagonists (asenapine, iloperidone, lurasidone, methysergide and olanzapine) to see by how much restoration of normal behavior can be achieved. The EC50 for each drug are determined for capacity to restore behavior. As negative control, the strain is exposed to FDA-approved agonist (vortioxetine) and inability to rescue function is measured. Next, a novel compound in clinical trials for antagonist are exposed to the promoter bashed strain (ly2590443) at the average EC50 concentration of known antagonists. Good candidates for drug development are revealed when candidate compounds are observed to cause restored behavior of the animal similar to the native-expressed hHTR7 strain.

### Example 5: Human Cannabinoid Receptor Transgenic C. Elegans

Provided herein are exemplary configurations of the present nucleic acid constructs each comprising a human cannabinoid receptor. The nucleic acid constructs prepared herein comprise a promoter regulatory sequence for ubiquitous expression in C. elegans neurons, a human cannabinoid receptor optimized for expression in C. elegans, and a 3′UTR in a plasmid designed for insertion into the worm genome.

A region of 1461bp immediately preceding the ATG of snb-1 neuronal promoter was amplified from the C. elegans genome using primers

GGGCTACGTAATACGACTCACTTAAGGCCTtaatcccaataaacctgtat
tcctgtgt/gtcgtcaagatggtcttatccgg(SEQ ID NO: 10)

(the uppercase sequence was used for Gibson ligation and the lowercase is specific for amplification).

Human cannabinoid receptor cDNAs (CNR1 (SEQ ID NO: 12) and CNR2 (SEQ ID NO: 14)) were optimized for expression in C. elegans using a four-step process: cDNA sequences were obtained from Ensembl (ensembl.org); cDNA codons were optimized optimal for expression in C. elegans using the C. elegans Codon Adapter (Redemann, et al. Nat Methods. 2011 Mar;8(3):250-23); synthetic introns were added to the cDNA sequence; and, the modified sequences were examined for proper splicing using NetGene2 (Hebsgaard, et al. Nucleic Acids Res., Vol. 24, No. 17, 3439-3452 (1996); Brunak, et al. J. Mol. Biol., 220, 49-65 (1991)). The appropriate Gibson ligation arms were added to the DNA sequence for cloning (Gibson et al. Nat. Methods May;6(5):343-5 (2009)).

The tbb-2 3′utr was used as a 3′ UTR that is permissive for expression. A region of 332 bp immediately following the Stop Codon of tbb-2 was amplified from the C. elegans genome using primer sequence GCCCTCTAAgataaatgcaaaatcctttcaagcattccc (SEQ ID NO: 10) /tgagacttttttcttggcggca (SEQ ID NO: 11). The uppercase sequence was used for Gibson ligation and the lowercase is specific for amplification. The plasmid backbone for this construct contained elements for growth and selection in E. coli the ColE1 origin and Ampicillin resistance. In addition, they contained elements for integration into the worm genome. pNU2006 contains homology arms for integration into Chromosome II at ttTi5605 using the MosSCI method (Frøkjaer-Jensen et al. Nat Genet. 40(11): 1375-1383 (2008)). pNU2007 contains homology arms for integration into Chromosome IV at cxTi10882 using the MosSCI method. Both constructs also contain an unc-119 selection marker from C. briggsae to allow identification of transgenic animals. These parts were assembled together using Gibson ligation and bacterial transformation. Bacterial colonies were observed, and a subset were grown in a liquid culture overnight. The plasmids were purified, and diagnostic restriction digest was performed to see if the plasmid had the expected size digest products. A plasmid showing the expected banding pattern in the diagnostic restriction digest was then sent out for sequencing (Eton Bio). AB1 files were provided and analyzed to determine that no mutations that would affect function were present.

The optimized coding sequence for human CNR1 optimized for expression in C. elegans is shown below:

ATGAAATCGATCTTGGATGGACTTGCAGATACTACGTTCCGTACCATCAC
CACCGACCTCCTCTACGTCGGATCCAACGACATCCAATACGAGGACATCA
AGGGAGACATGGCCTCCAAGCTCGGATACTTCCCACAAAAGTTCCCACTC
ACCTCCTTCCGTGGATCCCCATTCCAAGAGAAGATGACCGCCGGAGACAA
CCCACAACTCGTCCCAGCCGACCAAGTCAACATCACCGAGTTCTATAATA
AGTCCCTCTCCTCCTTCAAGgtaagtttaaacatatatatactaactaac
cctgattatttaaattttcagGAGAATGAGGAGAACATCCAATGCGGAGA
GAACTTCATGGACATCGAGTGCTTCATGGTCCTCAACCCATCCCAACAAC
TCGCCATCGCCGTCCTCTCCCTCACCCTCGGAACCTTCACCGTCCTCGAG
AACCTCCTCGTCCTCTGCGTCATCCTCCACTCCCGTTCCCTCCGTTGCCG
TCCATCCTACCACTTCATCGGATCCCTCGCCGTCGCCGACCTCCTCGGAT
CCGTCATCTTCGTCTACTCCTTCATCGACTTCCACGTCTTCCACAGAAAG
GACTCCCGTAACGTCTTCCTCTTTAAGCTTGGAGGAGTTACTGCCTCCTT
CACCGCTTCCGTCGGATCCCTCTTCCTCACCGCCATCGACCGTTACATCT
CCATCCACCGTCCACTCGCCTACAAGCGTATCGTCACCCGTCCAAAGgta
agtttaaaagttcgtactaactaaccatacatatttaaattttcagGCCG
TCGTCGCCTTCTGCCTCATGTGGACCATCGCCATCGTCATCGCCGTCCTC
CCACTCCTCGGATGGAACTGCGAGAAGCTCCAATCCGTCTGCTCCGACAT
CTTCCCACACATCGACGAGACCTACCTCATGTTCTGGATCGGAGTCACCT
CCGTCCTCCTCCTCTTCATCGTCTACGCCTACATGTACATCCTCTGGAAG
GCCCACTCCCACGCCGTCCGTATGATCCAACGTGGAACCCAAAAGTCCAT
CATCATCCACACCTCCGAGGACGGAAAGGTCCAAGTCACCCGTCCAGACC
AAGCTAGAATGGACATCCGTCTCGCCAAGgtaagtttaacatgattttac
taactaactaatctgatttaaattttcagACCCTCGTCCTCATCCTCGTC
GTCCTCATCATCTGCTGGGGACCACTCCTCGCCATCATGGTCTACGACGT
CTTCGGAAAGATGAACAAGCTCATCAAGACCGTCTTCGCCTTCTGCTCCA
TGCTCTGCCTCCTCAACTCCACCGTCAACCCAATCATCTACGCCCTCCGT
TCCAAGGACCTCCGTCACGCCTTCCGTTCCATGTTCCCATCCTGCGAGGG
AACCGCCCAACCACTCGACAACTCCATGGGAGACTCCGACTGCCTCCACA
AGCACGCCAACAACGCCGCCTCCGTCCACCGTGCCGCCGAGTCCTGCATC
AAGTCCACCGTCAAGATCGCCAAGGTCACCATGTCCGTCTCCACCGACAC
CTCCGCCGAGGCCCTCTAA (SEQ ID NO: 12)

The protein sequence for human CNR1 expressed in C. elegans is shown below:

MKSILDGLADTTFRTITTDLLYVGSNDIQYEDIKGDMASKLGYFPQKFPL
TSFRGSPFQEKMTAGDNPQLVPADQVNITEFYNKSLSSFKENEENIQCGE
NFMDIECFMVLNPSQQLAIAVLSLTLGTFTVLENLLVLCVILHSRSLRCR
PSYHFIGSLAVADLLGSVIFVYSFIDFHVFHRKDSRNVFLFKLGGVTASF
TASVGSLFLTAIDRYISIHRPLAYKRIVTRPKAVVAFCLMWTIAIVIAVL
PLLGWNCEKLQSVCSDIFPHIDETYLMFWIGVTSVLLLFIVYAYMYILWK
AHSHAVRMIQRGTQKSIIIHTSEDGKVQVTRPDQARMDIRLAKTLVLILV
VLIICWGPLLAIMVYDVFGKMNKLIKTVFAFCSMLCLLNSTVNPIIYALR
SKDLRHAFRSMFPSCEGTAQPLDNSMGDSDCLHKHANNAASVHRAAESCI
KSTVKIAKVTMSVSTDTSAEAL (SEQ ID NO: 13)

The optimized coding sequence for human CNR2 for expression in C. elegans is shown below:

ATGGAGGAATGTTGGGTTACAGAGATAGCAAATGGTAGCAAGGACGGACT
GATTCCAACCCTATGAAAGACTACATGATCCTCTCCGGACCACAAAAGAC
CGCCGTTGCCGTTCTCTGCACCCTCCTCGGACTCCTCTCTGCTCTTGAGA
ACGTCGCCGTCCTCTACCTCATCCTCTCCTCCCACCAACTCCGTCGTAAG
CCATCCTACCTCTTCATCGGATCCCTCGCCGGAGCCGACTTCCTCGCCTC
CGTCGTCTTCGCCTGCTCCTTCGTCAACTTCCACGTCTTCCACGGAGTCG
ACTCCAAGgtaagtttaaacatatatatactaactaaccctgattataaa
ttttcagGCCGTCTTCCTCCTCAAGATCGGATCCGTCACCATGACCTTCA
CCGCCTCCGTCGGATCCCTCCTCCTCACCGCCATCGACCGTTACCTCTGC
CTCCGTTACCCACCATCCTACAAGGCCCTCCTCACCCGTGGACGTGCCCT
CGTCACCCTCGGAATCATGTGGGTCCTCTCCGCCCTCGTCTCCTACCTCC
CACTCATGGGATGGACCTGCTGCCCACGTCCATGCTCCGAGCTCTTCCCA
CTCATCCCAAACGACTACCTCCTCTCCTGGCTCCTCTTCATCGCCTTCCT
CTTCTCCGGAATCATCTACACCTACGGACACGTCCTCTGGAAGgtaagtt
cctccactagaaatatcaggtgctataattgtgttcagGCCCACCAACAC
GTCGCCTCCCTCTCCGGACACCAAGACCGTCAAGTCCCAGGAATGGCCCG
TATGCGTCTCGACGTCCGTCTCGCCAAGACCCTCGGACTCGTCCTCGCCG
TCCTCCTCATCTGCTGGTTCCCAGTCCTCGCCCTCATGGCCCACTCCCTC
GCCACCACCCTCTCCGACCAAGTCAAGgtaagtttaacatgattttacta
actaactaatctgatttaaattttcagAAGGCCTTCGCCTTCTGCTCCAT
GCTCTGCCTCATCAACTCCATGGTCAACCCAGTCATCTACGCCCTCCGTT
CCGGAGAGATCCGTTCCTCCGCCCACCACTGCCTCGCCCACTGGAAGAAG
TGCGTCCGTGGACTCGGATCCGAGGCCAAGGAGGAGGCCCCACGTTCCTC
CGTCACCGAGACCGAGGCCGACGGAAAGATCACCCCATGGCCAGACTCCC
GTGACCTCGACCTCTCCGACTGCTAA (SEQ IDNO: 14)

The protein sequence for human CNR2 expressed in C. elegans is shown below:

MEECWVTEIANGSKDGLDSNPMKDYMILSGPQKTAVAVLCTLLGLLSALE
NVAVLYLILSSHQLRRKPSYLFIGSLAGADFLASVVFACSFVNFHVFHGV
DSKAVFLLKIGSVTMTFTASVGSLLLTAIDRYLCLRYPPSYKALLTRGRA
LVTLGIMWVLSALVSYLPLMGWTCCPRPCSELFPLIPNDYLLSWLLFIAF
LFSGIIYTYGHVLWKAHQHVASLSGHQDRQVPGMARMRLDVRLAKTLGLV
LAVLLICWFPVLALMAHSLATTLSDQVKKAFAFCSMLCLINSMVNPVIYA
LRSGEIRSSAHHCLAHWKKCVRGLGSEAKEEAPRSSVTETEADGKITPWP
DSRDLDLSDC (SEQ IDNO: 15)

Transgenic nematodes comprising CNR1 (SEQ ID NO. 12) or CNR2 (SEQ ID NO. 14) and expressing SEQ ID NO. 13 or SEQ ID NO. 15, respectively, can be crossed together to generate a new transgenic nematode comprising both SEQ ID NO. 12 (CNR1) and SEQ ID NO. 14 (CNR2). Males are generated from one of the strains and crossed with hermaphrodites from the other strain. The F1 generation is heterozygous for nematodes comprising coding sequences for both SEQ ID NO. 12 (CNR1) and SEQ ID NO. 14 (CNR2). F2 animals are selected and screened by PCR to identify animals homozygous for both SEQ ID NO. 12 (CNR1) and SEQ ID NO. 14 (CNR2).

An alternative method would be to create the lines sequentially, by generating one line and then genome editing in that line to insert the second sequence. Another possibility is to create an extrachromosomal array containing both SEQ ID NO: 12 (or encoding SEQ ID NO: 13) (CNR1) and SEQ ID NO. 14 (or encoding SEQ ID NO: 15) (CNR2).

### Example 6: Combination of C. Elegans Lines Comprising CNR1 and/or CNR2 with Other Transgenes

In some embodiments, the CNR1 and/or CNR2 transgenic C. elegans lines prepared as described herein can be genetically combined with other C. elegans strains (e.g., transgenic strains) to deplete the native endocannabinoid signaling pathway. These include strains lacking the endocannabinoid synthesis gene products such as phospholipase C beta (egl-8), diacylglycerol lipase (dagl-2), and N-acyl phosphatidylethanolamine-specific phospholipase-D (nape-1 and nape-2) (Harrison et al. PLoS One. 2014 Nov 25;9(11):e113007), and/or with strains lacking the native endocannabinoid receptors such as npr-19 (Oakes et al. J Neurosci. 37(11): 2859-2869 (2017)). Additionally, strains with altered ability to impair endocannabinoids are used such as those depleted in or over-expressing the fatty acid amide hydrolase (faah-2), which degrades and inactivates endocannabinoids (Harrison et al. PLoS One. 9(11):e113007 (2014)). Depletion of the native endocannabinoid signaling pathway components could provide increased sensitivity to exogenous cannabinoid signaling through the humanized CNR1 and CNR2.

In some embodiments, the CNR1 and/or CNR2 transgenic C. elegans lines prepared as described herein can be genetically combined with C. elegans strains comprising and expressing additional humanized proteins to identify cannabinoid treatment options for certain diseases. In one embodiment, human alleles are incorporated into the genome of a cell and/or animal in the nematode genome replacing a native allele for a gene with high homology, or in a different site of the genome, following standard methodologies (e.g., as described herein). In another instance, clinical variants of those human alleles are incorporated into the genome of a cell and/or animal into the human allele (e.g., STXBP1, KCNQ2, CACNB4, etc.) expressed in C. elegans, e.g., using genetic crosses of two different transgenic nematodes, and/or genome engineering. These multi-component humanized lines can be used in drug screening for cannabinoid effects on ameliorating phenotypes of the pathogenic disease genes. Drug activity on the humanized lines are compared to wildtype, null mutants, and the humanized controls with and without the CNR1 and CNR2 to determine if possible therapeutic effects are occurring. In some embodiments, the CNR1 and/or CNR2 transgenic C. elegans lines prepared as described herein can be genetically combined with C. elegans strains expressing the STXBP1 human protein variants, optimized for expression in the C. elegans, to identify cannabinoid treatment options for diseases such as epilepsy.

Pathogenic variants, S42P, R406H, R292H, and R388X, were introduced into the humanized STXBP1 line using a CRISPR/Cas9 system. These STXBP1 variants, before combination with a line comprising SEQ ID NO. 12 (CNR1) and SEQ ID NO. 14 (CNR2), result in phenotypic differences when compared to the wildtype human STXBP1 expressed in C. elegans in movement and morphology assessments. Data not shown.

### Example 7: Preparation of Present Nucleic Acid Constructs for Use in Zebrafish

Provided herein are exemplary configurations of the present nucleic acid constructs each comprising a human cannabinoid receptor, CNR1 and/or CNR2, for expression in zebrafish. The nucleic acid constructs comprise a promoter regulatory sequence for ubiquitous expression in Zebrafish neurons, human cannabinoid receptors optimized for expression in zebrafish, and a 3′UTR in a plasmid designed for insertion into the zebrafish genome. In some embodiments, the zebrafish neuronal promoter can be neurod1. In some embodiments, donor homology constructs can be designed using standard techniques such as by using Tol2 (Kawakami, et al. PNAS USA, 97, pp. 11403-11408 (2000)) for random insertion in the genome, and/or the CRISPR/Cas9 system for targeted insertion (Kimura, et al. Sci. Rep., 4: 1-7 (2014)). Targeted insertion can be directed to a “safe-harbor” site or to the native Zebrafish cnrl gene. An injection mix can be created with multiple nucleic acid and protein components, including the donor homology DNA and double-stranded break inducing component. Zebrafish embryos less than 4 hours post fertilization are typically injected, incubated to adulthood, and tested for germline transmission of the transgene. Animals homozygous for the genome edit are created by crossing using similar methodology using standard techniques (e.g., as described herein). Alternatively, point mutations modeling human variants may be incorporated into the genome of a cell and/or animal in the native Zebrafish cnrl or cnr2 genes. The resultant transgenic zebrafish can be used to screen for the effects of cannabinoids via phenotype read-out assays. Cannabinoid libraries can be generated de novo or purchased from commercial suppliers. Cannabinoids can be screened for synergistic or antagonistic activity toward alteration of normal phenotype using one or many phenotypic assays. In some embodiments, titrations at three (3)-fold doses (e.g., ranging from 1 mM to 128 mM) can be performed for each compound. After two hours of exposure, cohorts can be tested in one or more phenotypic assays, response curves plotted, and EC₅₀ values calculated. In some embodiments, such as for determining if antagonistic or synergistic activities are present between anti-epileptic drugs (AEDs) and cannabinoids, pairwise combinations can made followed by exposure of the humanized transgenic lines to such combinations. In mouse model assays (Smith, MD, Wilcox, KS, White, S. Analysis of Cannabidiol Interactions with Antiseizure Drugs. Annual Meeting American Epilepsy Society, 2015), carbamazepine (CBZ) was observed to antagonize a CBD, while levetiracetam (LEV) acts synergistically with CBD, and similar combinations can be tested in the transgenic zebrafish. In some embodiments, to observe antagonistic and synergistic effects, the concentration of CBD can be held at its observed EC₅₀ value in combination with titrations near the EC₅₀ of the AED drugs. In some embodiments, the phenotypic effect of CBZ can be suppressed while that of LEV enhanced. In other embodiments, other AED effects, if any, can be enhanced, suppressed, or remain unaltered.

### Example 8. Creation and Use of Transgenic C. Elegans Lines Expressing Therapeutic Targets and Disease Genes

In some embodiments, this disclosure provides transgenic animals (e.g., nematodes such as C. elegans, or zebrafish) and methods of preparing and using same for expression and drug discovery for therapeutic targets, wherein the therapeutic target gene and disease gene are first optimized for expression in a host animal and then incorporated into the genome of the animal. By way of example to demonstrate the general principle of expressing therapeutic targets in combination with disease genes in the nematode, the human coding sequence for gamma-aminobutyric acid transaminase (hGABAT) is inserted into the C. elegans genome. hGABAT is an important target for anti-epilepsy drugs such as valproic acid and vigabatrin even when a variant in hGABAT is not present in the treated patient. Literature evidence can be found of the effectiveness of vigabatrin for patients with hSTXBP1 variants (Romaniello et al. 29(2): 249-253 (2013)), hKCNQ2 variants (Lee, et al. Pediatr. Neurol. 40(5):387-91 (2009)), and hCDKL5 variants (Melikishvili G, et al. Epilepsy Behav. 94:308-311 (2019)), among others. In one embodiment, the human hGABAT coding sequence is optimized for expression in C. elegans. Artificial C. elegans introns are added and aberrant splice sites are removed. For hGABAT, the following expression-optimized cDNA sequence with introns inserted (lower case) is used:

ATGGCCTCCATGCTCCTCGCCCAACGTCTCGCCTGCTCCTTCCAACACTC
CTACCGTCTCCTCGTCCCAGGATCCCGTCACATCTCCCAAGCCGCCGCCA
AGGTCGACGTCGAGTTCGACTACGACGGACCACTCATGAAGACCGAGGTC
CCAGGACCACGTTCCCAAGAGCTCATGAAGCAACTCAACATCATCCAAAA
CGCCGAGGCCGTCCACTTCTTCTGCAACTACGAGGAGTCCCGTGGAAACT
ACCTCGTCGACGTCGACGGAAACCGTATGCTCGACCTCTACTCCCAAATC
TCCTCCGTCCCAATCGGATACTCCCACCCAGCCCTCCTCAAGCTCATCCA
ACAACCACAAAACGCCTCCATGTTCGTCAACCGTCCAGCCCTCGGAATCC
TCCCACCAGAGAACTTCGTCGAGAAGCTCCGTCAATCCCTCCTCTCCGTC
GCCCCAAAGgtacttgagatccttaaacgcagtcgaaaattggtaatttt
acagGGAATGTCCCAACTCATCACCATGGCCTGCGGATCCTGCTCCAACG
AGAACGCCCTCAAGACCATCTTCATGTGGTACCGTTCCAAGGAGCGTGGA
CAACGTGGATTCTCCCAAGAGGAGCTCGAGACCTGCATGATCAACCAAGC
CCCAGGATGCCCAGACTACTCCATCCTCTCCTTCATGGGAGCCTTCCACG
GACGTACCATGGGATGCCTCGCCACCACCCACTCCAAGGCCATCCACAAG
ATCGACATCCCATCCTTCGACTGGCCAATCGCCCCATTCCCACGTCTCAA
GTACCCACTCGAGGAGTTCGTCAAGgtaagttcctccactagaaatatca
ggtgctataattgtgttcagGAGAACCAACAAGAGGAGGCCCGTTGCCTC
GAGGAGGTCGAGGACCTCATCGTCAAGTACCGTAAGAAGAAGAAGACCGT
CGCCGGAATCATCGTCGAGCCAATCCAATCCGAGGGAGGAGACAACCACG
CCTCCGACGACTTCTTCCGTAAGCTCCGTGACATCGCCCGTAAGCACGGA
TGCGCCTTCCTCGTCGACGAGGTCCAAACCGGAGGAGGATGCACCGGAAA
GTTCTGGGCCCACGAGCACTGGGGACTCGACGACCCAGCCGACGTCATGA
CCTTCTCCAAGAAGATGATGACCGGAGGATTCTTCCACAAGgtgagttat
tataatttttttgatcacaacgattattttaattttcagGAGGAGTTCCG
TCCAAACGCCCCATACCGTATCTTCAACACCTGGCTCGGAGACCCATCCA
AGAACCTCCTCCTCGCCGAGGTCATCAACATCATCAAGCGTGAGGACCTC
CTCAACAACGCCGCCCACGCCGGAAAGGCCCTCCTCACCGGACTCCTCGA
CCTCCAAGCCCGTTACCCACAATTCATCTCCCGTGTCCGTGGACGTGGAA
CCTTCTGCTCCTTCGACACCCCAGACGACTCCATCCGTAACAAGCTCATC
CTCATCGCCCGTAACAAGGGAGTCGTCCTCGGAGGATGCGGAGACAAGTC
CATCCGTTTCCGTCCAACCCTCGTCTTCCGTGACCACCACGCCCACCTCT
TCCTCAACATCTTCTCCGACATCCTCGCCGACTTCAAGTAA (SEQ ID
NO: 16)

The hGABAT sequence is inserted into the C. elegans genome at the orthologous gene locus, gta-1, using CRISPR/Cas9. The hSTXBP1 sequence is inserted into the C. elegans genome at the orthologous gene locus, unc-18, using CRISPR/Cas9 using methods described in WO 2019/165128. The C. elegans genes gta-1 and unc-18 have overlapping tissue expression so native orthologous expression can be used, but in cases where the expression is not overlapping alternative promoters and transgenic methods are used. The insertion of hGABAT is combined with the insertion of hSTXBP1 to create the double humanized model. Pathogenic variants are modeled in hSTXBP1 using CRISPR/Cas9 to create double humanized patient allele models. Movement, morphology, electrophysiology, growth rate, and other phenotypic measures are used to characterize the double humanized patient allele models. Double humanized patient allele models are treated with compounds targeting hGABAT and are characterized by phenotyping. Compounds that bring the phenotype of the double humanized patient allele models closer to the wild-type double humanized model are potential hits. Those that do not improve the hSTXBP1 animal that does not contain the hGABAT or that has the native gta-1 also knocked out are candidates for directly targeting hGABAT. In some embodiments, a double humanized patient allele model can be developed by selecting one of the combinations of Therapeutic Target Gene and a different Human Disease Gene shown above in Table 6.

### Example 9: Methods for In-Silico Compound Screening

This example describes methods for using a humanized C. elegans animal model combined with patient human induced pluripotent stem cells (hIPSC)-derived in a neuronal assay for rapid validation of in-silico drug screening data to identify candidate compounds with therapeutic potential. In this example, the pathogenic R406H variant of STXBP1 (STXBP1(R406H)) was studied. Syntaxin binding protein 1 (STXBP1) plays an important role in presynaptic vesicle docking and fusion, and thus an essential component in the neurotransmitter secretion mechanism (Toonen, et al. Trends Neurosci. 30: 564-72 (2007)). STXBP1 related disorders are collectively termed STXBP1-encephalopathy (STXBP1-E), these are neurodevelopmental disorders displaying diverse clinical features including epilepsy (~95% of patients), different movement disorders, intellectual disabilities (ID) and autistic features (Stamberger, et al. Neurology 86: 954-62 (2016)). Pathophysiology is caused by variations in STXBP1 gene, such variations including missense, nonsense, frameshift, and splice site changes, and/or whole gene deletions. Current treatment for STXBP1-E patients is limited to amelioration of symptoms (e.g., using physiotherapy, speech therapy, and/or occupational therapy) and seizure control, which is not fully effective for all patients and almost one in three remain therapy-resistant. To identify therapeutics that act directly to reverse the deficiencies caused by a pathogenic variation in STXBP1, the humanized C. elegans animal model has been used in this example. Briefly, a nucleic acid encoding STXBP1(R406H) (i.e., the STXBP1(R406H) transgene) was introduced into the C. elegans genome. When pathogenic behavior was detected in the humanized C. elegans, and a library of compounds screened to identify therapeutic candidates capable of restoring non-pathogenic (i.e., normal, wild-type) behavior. Assays that can be used in these comparisons can include but are not limited to the worm locomotion assay (Brenner, S., et al. Genetics 77: 71-94 (1974)) and the aldicarb sensitivity assay (Martin, et al. Curr. Biol. 21: 97-105 (2011)), among others. The aldicarb assay, for example, can be used to directly monitor the capacity of a test compound to restore evoked release of acetylcholine from presynaptic termini. Knock-out unc-18 C. elegans animals lacking their ortholog version of the STXBP1 gene are highly resistant to aldicarb-induced paralysis. Aldicarb acts by blocking acetylcholinesterase which leads to an overstimulation of the post-synaptic terminus. The loss of unc-18 prevents this over-stimulus because synaptic vesicle release is greatly reduced in the unc-18 null animals. The result is unc-18 animals are highly resistant to aldicarb overstimulation (i.e., stabilizing STXBP1). It has been shown that when trehalose was exposed to a strain containing the R406H variant, the levels of paralysis was observed to increase to wild-type levels (Guiberson, et al. Nat. Commun. 9: 3986 (2018)). Briefly, these exemplary methods can include performing in-vivo testing in STXBP1(R406H) humanized animal for activity on in silico derived hits of FDA approved compounds; determining if activity is conserved by screening the in-vivo validated and non-validated in silico hits for ability to improve neuronal function of iPSC-derived neurons from a STXBP1(R406H) patient; and, using in-vivo screening results to refine the in-silico approach and select more drug candidates for activity on R406H (i.e., a repetitive or iterative process) (illustrated in FIG. 1). The exemplary in-vivo screening steps disclosed herein can be used refine in-silico approach and select more drug candidates for activity on R406H for testing back in-vivo. Hit expansion can be carried out in order to explore a wider chemical space, and the biological results used to identify key features of the potential hits, enabling us to identify molecules with higher probability to have the desired therapeutic effect. Multiple rounds of discovery and refinement can be deployed to explore pharmacophore optimization. The potential validation of the in-silico/ in vivo/human-IPSC modeling pipeline for personalized drug repurposing proposed here for mutation R406H could be further used as a personalized approach that is cost-effective and mutation specific for other STXBP1 variants, thereby dramatically shortening the period between experimentally identifying a potential candidate drug and testing it clinically on STXBP1 patients.

In-silico Prediction. Computer-aided drug design is a fast and efficient approach to drug discovery (Macalino, et al. Arch. Pharm. Res. 38: 1686-1701 (2015)). Molecular dynamics (MD) simulations were applied in order to predict the effect of the R406H mutation on structure and function of STXBP1. The high-resolution X-ray crystal structure (PDB: 4JEH) www.rcsb.org/structure/4JEH was used as a starting point of the MD simulation. Analyzing the MD extracted trajectories, influence of the mutation on protein conformation and function is similar to that observed by Bar-On and colleagues (Bar-On, et al. PLoS Comput. Biol. 7: e1001097 (2011)). A cavity at the vicinity of residue 406 that is formed as a result of the mutation due to a missing hydrogen-bond was observed. It was hypothesized that binding a small molecule at that cavity may fill the gap created and rescue the protein function. Representative conformations were retrieved from the MD simulations trajectories, and these structures used for structure-based virtual screening (i.e., docking studies) of comprehensive libraries of approved drugs, natural products, and drugs that reached various phases of clinical development (identified in the Spectrum Collection and Broad Institute repurposing databases). These studies identified approximately 100 potential hits (i.e., that virtually bind to the mutated site and improve the protein structure stability). An example of a small molecule docked to STXBP1-R406H is shown in FIG. 4. A subset including 20 of the approximately 100 candidates in silico-predicted drugs/natural products are phenotypically tested on the R406H STXBP1 variant strain for capacity to restore normal behavior in the humanized C. elegans animals.

Humanization Process. The process for making the humanized C. elegans animal model for the R406H STXBP1 variant strain includes three steps: 1) the native gene (unc-18) can be removed (i.e., deleted) from the animal genome or amino acid(s) can be changed at conserved positions in the nematodes’s unc-18 ortholog version of the STXBP1 gene (the “native locus”), resulting in a severely uncoordinated animal (i.e., pathogenic behavior, pathophysiology); 2) the removed DNA sequence is replaced with the human STXBP1 gene sequence, which results in almost complete rescue-of-function; and, 3) the R406H variation is inserted into the STXBP1-humanized locus after which the animal is characterized to identify any pathogenic behavior (e.g., a severely uncoordinated animal). Functional tests used are typically high-dimensional phenotypic studies (e.g., electrophysiology, motion, morphology and gene expression) that allow quantitation of a variety of phenotypic deficiencies in transgenic animals. The functional testing of R406H in the STXBP-humanized locus shows clear correlation to the human disease phenotype. Use of human sequences as a backbone for variant installations appears to sensitize the animal for detection of known pathogenic missense variants (STXBP1(R292H) and STXBP1(R406H)). Humanization can be accomplished by introducing amino acid at conserved positions in the nematodes’s unc-18 ortholog version of the STXBP1 gene (“Native locus”); or, expressing the entire human STXBP1 gene as a replacement of the C. elegans native unc-18 coding sequence.

Aldicarb Sensitivity Assay. In some embodiments, a liquid aldicarb sensitivity assay can be performed by introducing transgenic animals to one or more compounds in 96-well format, followed by monitoring over time for aldicarb induced loss of motion by use of a WMmicrotracker Apparatus (Nemametrix, Inc). In some embodiments, three-fold titrations of compound starting at 100 mg/ml for 12 wells can be used to test a five (5) order-of-magnitude concentration range. Suspensions of animals can be introduced in parallel to the serial dilutions and the activity of the animals monitored across 20-minute intervals for two (2) hrs. Plots of sensitivity to aldicarb-induced paralysis can indicate which compounds are candidates for further analysis. Compounds of potential interest are those that re-sensitize the animals to aldicarb exposure. To filter out non-specific paralysis, hits found on the STXBP1(R406H) strain can then be rescreened on the STXBP1(WT) strain. Hits with strong ratio of R406H/WT activity can then advance to human iPSC cell activity detection.

iPSC Cells and Assays. As mentioned above, induced pluripotent stem cell (iPSC) can be used to reprogram patient-derived somatic cells into an embryonic stem cell-like state followed by a differentiation to the disease-relevant cell type. This technology has proven to be a powerful tool for modeling diseases and for drug screening (Takahashi, et al. Cell 131: 861-872 (2007); Lee, et al. Nat. Biotechnol. 30: 1244-8 (2012)) and was previously been implemented in disorders similar to STXBP1 (e.g., Rett syndrome and Dravet syndrome (Marchetto, Cell, 143(4), 527-539 (2010); Schuster, et al. Neurobiology of Disease, 132, 104583 (2019)). The use of iPSC-derived neurons from R406H patient allows a unique opportunity for testing either the in silico drug hits directly on the cells or after their previous validation by the worm in vivo. In addition to testing for functional improvement of the iPSC-derived neurons, this tool will provide for exploring the potential rescue mechanism of positive hits and gaining a more thorough understanding of pathological mechanisms underlying the patient symptoms and improve patient treatment’s precision. Thus, producing viable and functional neurons from STXBP1(R406H) patient-derived IPSC will provide for screening of in silico-derived and/or worm in-vivo validated hits and test the ability of compounds identified thereby to improve neuronal function/phenotype in the human cell model. In some embodiments, primary fibroblasts can be isolated from a skin biopsy of a human patient. Viable iPSC and isolated stem cell colonies will be selected based on genomic stability and pluripotential capacity. The specific capacity to produce functional cortical neurons can then be tested. Those cells identified as having such capacity can be used in functional neuronal assays to test the drugs/natural products by their potential to improve in vitro electrophysiological activities and neurotransmitters release, in a similar manner as previously used for phenotyping STXBP1 variants containing neurons (Yamashita, et al. Epilepsia 57: e81-e86 (2016); Kovačevic, et al. Brain (2018)).

Exemplary Results. An 8000-compound library was studied by in silico methods to derive a rank of compounds for likeliness to improve the stability of R406H. Twenty compounds are examined for capacity to restore function in a humanized STXBP1 animal harboring the R406H variation.

### Example 10. Creation of Drug Screen Platform for Alzheimer Related Dementias

This example describes reagents and methods for identifying therapeutic compounds for reversing pathogenic behavior in genetic variants associated with Alzheimer Dementia Related Disorders (ADRD), which affects more than five million people in the US (1.5% of population). A variety of genes are involved in the defects that lead to neurodegeneration and shortened lifespan including but not limited to MAPT, GRN, TARDBP, APP, PSEN1, and PSEN2. Orthologs to these genes exist in C. elegans (ptl-1, pgrn-1, tdp-1, apl-1, sel-12, hop-1, respectively) and a variety of prior studies indicate defects in these genes influence neuronal integrity, elevate calcium signaling, and reduce lifespan in C. elegans (Chew et al., J Cell Sci, 2013; Salazar et al., J Neurosci, 2015; Ewald et al., Aging Cell, 2016; Sarasija and Norman, Genetics, 2015; Caldwell; Dis Model Mech; 2020). Reporter systems exist in C. elegans for sensing neuronal integrity (Martinez et al., J Neurosci, 2017), calcium signaling (Sarasija and Norman, Genetics, 2015)) and reduced lifespan (Mendenhall et al., J Gerentol A Biol Sci Med Sc, 2017), but are confounded by high background interference and a need for attenuation with specific genetic backgrounds. To circumvent these problems and enable easier automation of activity assessment, the methods described in this example combine multiple genetic expression outputs to yield activity assays that are more amenable to high-throughput screening.

In some embodiments, split green fluorescent protein (GFP) is used for precision tissue labeling (“split fluor technique”) as it reduces background signal. For instance, in some embodiments, a tissue specific promoter is used to express GFP1-10 in one or more particular tissue(s) for which the tissue specific promoter (TSP) drives gene expression. GFP1-10 is non-fluorescent due to a lack of the 11th beta sheet that is needed for hydrogen bond stabilization of the fluorescent chromophore. To create bimolecular fluorescence complementation, a GFP11 peptide is co-expressed with GFP1-10 to enable a quenched chromophore environment and enable fluorescence emission upon exposure to the appropriate excitation wavelength. GFP1-10 protein is expressed in the tissues in which the tissue-specific promoter is active (i.e., drives gene expression). A different or non- tissue-specific promoter can be used to drive GFP11 in a different subset of tissues (e.g., all tissues of the organism). In the tissues where expression of GFP1-10 and GFP11 overlap, co-expression of GFP1-10 and GFP11 will occur leading to fluorescent labeling of those tissues. Similar approaches can be done with other split GFP systems such as sfCherry. For instance, Phsp-6::sfCherry is a biomarker reporter construct indicative of the mitochondrial stress response and is expressed in target neurons as well as in the pharynx and gut. To achieve neuron-specific expression, Phsp-6::sfCherry1-10 (driving expression of GFP1-10 in dopaminergic neurons) is combined with a Pdat-1::sfCherry11 (driving expression of GFP11), where overlapping activity the Phsp-6 and Pdat-1 promoters occurs exclusively in dopaminergic neurons such that both GFP1-10 and GFP11 are both expressed in those cells (FIG. 5A). Alternatively, in some embodiments, tissue-specific expression of a stress reporter is targeted to all neurons via genetic cross of a C. elegans strain Phsp-6::sfCherry1-10 strain into a C. elegans strain harboring Psnb-1::sfCherry11. CRISPR-based gene editing is used to introduce clinical variants of one or more ADRD gene(s) into either native or gene-humanized C. elegans loci.

In one embodiment, the human sequence for human TARDBP human TARDBP is inserted into the genome as a transgene at a safe-harbor locus and its expression was driven by a heterologous neuronal promoter (Psnb-1::hTARDBP::eft-3u). In another embodiment the human TARDB is inserted into the native locus with the same sequence (Psnb-1::hTARDBP::eft-3u). In another embodiment, the TARDBP was inserted into the native locus without the heterologous neuronal promoter and terminator sequence. Three clinically-observed pathogenic variants of TARDBP (G294A, G295S and/or G298S) are installed in the humanized TARDBP locus. Functional testing on the wild-type and pathogenic variants indicated loss of cholinergic signaling was observed in G294A and G295S but not G298S by assessment of paraquat hypersensitivity in a dye-filling assay of amphid neurons (FIG. 5B). As a complement, neuronal degradation can also be monitored via a thrashing assay to detect locomotion defects (Wang et al. PLoS Genetics, 2009). For drug screening, test compounds can be tested in these tissue-specific expression lines for their capacity to suppress the Phsp-6 stress reporter response and the resulting hits are validated by testing thrashing (phenotype) assays for restoration of normal activity.

In a similar manner, in some embodiments, pathogenic variants of MAPT (e.g., G272V and P301L) in a humanized C. elegans MAPT line are bred into the reporter background (i.e., C. elegans organisms expressing GFP11 in all tissues) and monitored for changes in stress reporter responses in the presence or absence of one or more test compounds.

In some embodiments, genetically encoded calcium indicators (GECls) are used to uncover altered calcium signaling. For instance, in some embodiments, a Psnb-1 promoter is fused to a GCaMP7 (or similar GECI) to create a Psnb-1::GCaMP7 construct. The Psnb-1::GCaMP7 construct is integrated into the genome of C. elegans using CRISPR techniques. Fluorescence monitoring of the Psnb-1::GCaMP7 strain is used to derive a baseline response for wild-type. The strain is then genetically crossed into an appropriate C. elegans strain such as hTARDBP-wt and hTARDBP-G295S (or hTARDBP-G294A or hTARDBP-G298S) followed by monitoring for altered calcium signaling.

In some embodiments, a hTARDBP-wt is incorporated into the zebrafish genome to replace the zebrafish homologs tardbp or tardbpl locus, and clinical variants (e.g., hTARDBP-G295S, hTARDBP-G294A, or hTARDBP-G298S) are installed into this humanized locus (hTARDBP-wt). Concomitantly, a neuronal (e.g., elavl3) or glial (e.g., gfap) tissue-specific promoter is operably linked to a GECI (e.g., GCaMP6) sequence and inserted into the zebrafish genome by either random integration (tol2 integrase) or safe-harbor site (phC31 integrase) to provide tissue-specific GECI reporters. Zebrafish containing the transgene (elavl3:GCaMP6 or gfap:GCaMP6). The hTARDBP strains are bred into the GECI reporter strains (or vica versa) which can then be monitored for altered calcium signaling due to defects in the TARDBP clinical variants. For drug screening, compounds can be tested in these tissue specific reporter lines for their capacity to restore normal calcium signaling (i.e., correct for the TARDBP clinical variant defect(s)).

In some embodiments, MAPT variants (G272V and P301L) in a humanized MAPT line are brought into the reporter background (strain expressing GFP11) and monitored for changes in calcium reporter responses in the presence or absence of the test compound(s).

In some embodiments, shortened lifespan due to neurodegeneration is monitored via automated lifespan analysis. A life-span/health-span instrument was adapted as a modified document scanner to enable repetitive imaging of a plate series throughout the lifespan of the transgenic C. elegans animal models. By plotting the series of images as a function of time to loss of activity for each animal can be determined. In some embodiments, techniques are applied to images can uncover movement rates and behaviors that correlate with quality of health during lifespan (“healthspan”). Applied to both the C. elegans humanized hTARDBP and hMAPT strains described above, the behavioral activity throughout the life of the organisms can be used to profile differences in variant activity which then can be used as biomarkers for drug studies on finding compounds that reverse anomalous lifespan behavior back to normal.

In some embodiments, expression profiling using transcriptomics and proteomics is used to detect shared mechanisms of dysfunction in gene variants involved in Alzheimer’s-related dementias. For instance, in some embodiments, RNAseq is used to assay the transgenic TARDBP and MAPT C. elegans to create a profile of the changes in transcription and translation protein interactions. The profile changes are mapped to nematode-customized ontologies that provide nodal foci of signaling pathway intersections. Disruption of signaling nodes in the nematode ontologies that have been cross referenced to human ontologies can provide an indication of mechanism of action in humans. Drug compound responses that lead to normal signaling strength in data flow through the ontologies. Restoration of normal ontology vector strength provides evidence for return to normal mechanistic function in the humanized variants harboring genetic lesions.

### Example 11. Reagents and Methods for Studying Infectious Disease

This example describes reagents and methods for studying viral receptor engagement, viral entry into host tissues, and viral transcriptional responses in living organisms such as C. elegans and zebrafish. In some embodiments, the human ACE2 receptor and the TMPRSS2 cofactor are inserted into the C. elegans genome as a single copy, under the control of a tissue-specific promoters such as vha-6 for expression in the intestinal tissues using the MosSCI (Mos1-mediated Single Copy Insertion) method of transgenesis (Christian Frøkjaer-Jensen, et al., Single copy insertion of transgenes in C. elegans, Nat Genet. 2008 Nov; 40(11): 1375-1383. MosSCI transgenesis method inserts genetic cargo at defined locations in the C. elegans genome using unc-119 rescue cassette insertion to bring genetic cargo (e.g., human homolog and/or clinical variant thereof) into a target locus (e.g., a C. elegans or zebrafish homologue of the clinical variant), creating a rescue of function on the unc-119(ed3) III mutant allele. Targeting of cargo insertion occurs at select Mos1 loci. Each Mos1 locus used has been selected for position neutral effects and avoids the intragenic region of gene coding, introns, and transcription factor binding sites. The transgenic organisms (e.g., nematodes) created have the genotype [vha-6::TMPRSS2::GFP::tbb-2utr, unc-119(+))] II , unc-119(ed3) III] or [vha-6p::hACE2::mCherry::tbb-2utr, unc-119(+))] II, unc-119(ed3) III]. Validation using any suitable technique (e.g., PCR) is then performed to confirm the presence of the transgene in the organism (e.g., C. elegans or zebrafish), and fluorescent images confirming the protein-fluorophore fusion expression are obtained (FIG. 6A). The transgenic strains produced as described herein are used as created or crossed to produce worms expressing both the human viral receptors and any necessary co-factors for entry or replication. The resultant animals are used in combination with exogenous biologic compounds such as live virus, pseudovirus, chimeric virus, viral fragments, and/or mRNA coding viral fragments to determine whether these exogenous biologics engage with the human viral receptors, and whether they are endocytosed and replicate in the C. elegans tissue. Because RNAi limits viral replication, the viral receptor and co-factor strains may be crossed with the rde-1 strain of C. elegans wherein RNAi machinery is defective to allow efficient replication. The resultant system is used to determine whether candidate antigens interact with the viral receptors and elicit a cellular response. The system is a format in which the receptors, receptor variants and/or the antigens can be modified to provide a robust readout of receptor engagement and entry.

The resultant system comprising (a) animal models expressing viral-interacting proteins and biologics containing viral elements is then used to screen for compounds that may inhibit viral engagement, endocytosis, replication. RNAseq can also be used to determine a transcriptional map of pro-viral and anti-viral response genes. Compounds can then be screened for anti-correlated transcriptional responses, indicating that compounds are effective in attenuating viral infection by increasing immune responses.

In some embodiments, ACE2-humanized transgenic zebrafish are created as described herein for use as a high-throughput infection model for studying COVID-19 disease. The method for transgene insertion can use a combination of CRISPR and phiC31 integrase activity. The phiC31 integrase technology can provide advantages because, unlike CRISPR based methods, the phiC31 integrase has a proven capacity to insert large segments of DNA content (~8kb) and at the high germline efficiency of up to 10% of an injected clutch of embryos. A two-step process is used in creation of a humanized line. First, a germline knock-out is made by introduction of an attP-stop in the first exon of ACE2 (the “attP-stop strain”). Next, the attP-stop strain is used as substrate for knock-in insertion of human ACE2 cDNA (the “attP-stop ACE2 strain”). The germline knock-out (“attP-stop KO”), a small donor homology ODN brings in the 50 bp attP sequence and its inherent stop codon. Integration of this attP-stop site requires a pair of sgRNA/Cas9 nuclease sites arranged in a PAM-out configuration such that a 74 bp region is removed from the first exon of ace2. Cellular double-strand break repair machinery is used to repair the region with either Non-Homologous End Joining (NHEJ) or Homology-Directed Repair (HDR). Allele-specific PCR (ASPCR) is used to detect if the desired HDR editing event has occurred. F0 animals with highest attP-stop signal in soma will be crossed with wild-type zebrafish. F1 embryos positive by the ZEG assay for attP-stop will be grown to adulthood and crossed again into wild-type to produce the F2 progeny (+/- heterozygote) that will be used in the construction of the humanized ACE2 strain. The creation of ACE2 knock-in (“hACE2 KI”), mRNA for phiC31-nanos1-3′UTR is used to provide transient expression of the integrase. Co-injection of the integrase with a plasmid containing a 50 bp attB sequence (CGGTGCGGGTGCCAGGGCGTGCCCTTGGGCTCCCCGGGCGCGTACTCCAC (SEQ ID NO: 103)) targets gene insertion to occur with the reading frame of the gene remaining intact. A P2A self-cleaving peptide is co-introduced between native and introduced cDNA sequence, which results in ACE2 cDNA being expressed with the addition of only one proline at the N terminus. ZEG (zebrafish embryo genotyper) assay with ASPCR will be used to find injected embryos with high levels of desired gene insertion. Animals with the highest ASPCR signals are grown to adulthood and crossed into wild-type zebrafish. The resulting F1 cross progeny are screened by ZEG and progeny positive for ACE2 gene insertion (+/- heterozygote) by ASPCR become founder animals for further studies. FIG. 6A provides genomic map for the human ACE2 insertion construct. The homology arms are in tan. The unc-119 promoter is in grey. The unc-119 coding sequence is in green. The unc-119 3′ UTR is in dark blue. The vha-6 promoter is in light blue. The human ACE2 sequence is in purple. The mCherry is in red. The tbb-2 UTR is in dark blue. FIG. 6B provides PCR data confirming the presence of the desired edit in candidate C. elegans strains. FIG. 6C provides fluorescent image confirming the presence of the hACE2::mCherry fusion in the intestinal tissues of the C. elegans organism.

Other uses for such methods are also contemplated herein as would be understood by those of ordinary skill in the art.

While certain embodiments have been described in terms of the preferred embodiments, it is understood that variations and modifications will occur to those skilled in the art. Therefore, it is intended that the appended claims cover all such equivalent variations that come within the scope of the following claims.

We claim:

1. A non-human transgenic organism for assessing the interaction of a human therapeutic agent and a therapeutic target,
wherein the non-human transgenic host organism comprises a human heterologous gene encoding a protein sequence for the therapeutic target operably linked to a heterologous promoter selected for expression in the host organism cells,
wherein:
the human heterologous gene is inserted into a non-native locus of the host organism’s genome, and
expression of the human heterologous gene is expressed in non-orthologous time and/or non-orthologous tissue.

2. The transgenic organism of claim 1, wherein the organism is a nematode or zebrafish.

3. The transgenic organism of claim 1 or 2, wherein the therapeutic target is over expressed in non-orthologous tissue.

4. The transgenic organism of any preceding claim, wherein the therapeutic target comprises a clinical sequence variant, is a receptor, is a viral receptor, or is a G-protein coupled receptor associated with disease in humans.

5. The transgenic organism of any preceding claim, wherein the therapeutic agent comprises a compound, small molecule, and/or biologic component.

6. The transgenic organism of claim 5, wherein the biologic component is mRNA.

7. The transgenic organism of claim 1 wherein the human heterologous gene is a chimeric sequence comprising heterologous exon coding sequences interspersed with artificial host organism intron sequences optimized for expression in the host nematode.

8. A method of generating and/or assessing a non-human transgenic organism for assessing the interaction between a human therapeutic agent and a therapeutic target, wherein the transgenic organism has an increased sensitivity to the human therapeutic agent, the method comprising:
a) selecting a target sequence comprising therapeutic target protein coding sequence;
b) selecting a tissue-specific and/or time-specific regulatory sequence as a combination of a promoter sequence and a downstream untranslated region;
c) combining the sequences by fusing the regulatory sequence to the target sequence;
d) creating a non-human transgenic organism by inserting the combined sequence into a non-native locus of the genome of the non-human transgenic organism; and,
e) optionally contacting the transgenic organism to the therapeutic agent and observing an elevated phenotypic response due to the activity of the transgene.

9. A method for assessing the interaction between a human therapeutic agent and a therapeutic target over-expressed in a non-human transgenic organism for increased sensitivity of the host organism to the human therapeutic agent, the method comprising:
a) providing a non-human transgenic organism of claim 1 comprising at least one of a human heterologous sequence expressing a therapeutic target providing a modified phenotype to the organism that is distinguished from a non-genetically modified host organism phenotype in at least one statistically significant measurable difference;
b) contacting the genetically modified host organism of step a) with one or more human therapeutic agent(s) during an incubation period;
c) performing one or more phenotype assay(s), during or after the incubation period, to assess interaction of the human therapeutic agent and overexpressed therapeutic target; and,
d) recording a change in the modified phenotype following the phenotype assay, whereby, human therapeutic agents are assessed and selected based on their change in the modified phenotype.

10. The method of claim 8 or 9 wherein expression of the therapeutic target in the non-human transgenic organism has a quantifiable phenotype that differs from the wildtype non-human organism.

11. The method of any one of claims 8-10wherein the therapeutic target is one previously identified via in-silico modeling, biochemical assay, or systems-level transcriptomic assay.

12. The method of any one of claims 8-11, wherein the phenotype is measured as fluorescence of an exogenous reporting molecule, mRNA expression optionally assayed via RNAseq and/or microarray, the lifespan of the organism, or protein expression.

13. The method of any one of claims 8-12wherein the phenotype is revealed via response to exposure to an exogenously applied agent comprised of chemical enhancer or repressor, virus, bacterium, or pseudo- or chimeric virus.

14. The method of any one of claims 8-13wherein exposure of the non-human transgenic animal to the therapeutic agent results in an anti-correlated phenotype to the non-human organism expressing the therapeutic target, with or without exposure to the phenotype-revealing agent.

15. A nucleic acid construct comprising a chimera of a C. elegans daf-12 DNA binding domain and a ligand binding domain of a nuclear hormone receptor operably linked to the coding sequence of a fluorescent reporter molecule.

16. A C. elegans animal comprising the nucleic acid construct of claim 15 incorporated into its genome.

17. A method of screening human therapeutic agents that target one or more nuclear hormone receptors, the method comprising:
a) treating a transgenic C. elegans animal of claim 16 with a potential test compound; and,
b) observing the phenotypic response of the transgenic C. elegans animal; wherein the phenotypic response indicates whether the test compound is an agonist or antagonist of the nuclear hormone receptor.

18. The method of claim 17 wherein the nuclear hormone receptor is selected from the group consisting of VDR, RXRA, ESR1, PPARG, AHR, RARA, RARB, RARG, PPARA, PPARD, NR1D1, NR1D2, RORA, RORB, RORC, NR1H3, NR1H2, NR1H4, NR1H5P, NR1l2, NR1l3, HNF4A, HNF4G, RXRB, RXRG, NR2C1, NR2C2, NR2E1, NR2E3, NR2F1,NR2F2, NR2F6, ESR2, ESRRA, ESRRB, ESRRG, NR3C1, NR3C2, PGR, AR, NR4A1, NR4A2, NR4A3, NR5A1, NR5A2, NR6A1, NR0B1, and NR0B2.

19. A nucleic acid construct comprising a human GPCR gene operably linked to a native promoter of the human GPCR gene ortholog of a non-human animal.

20. The nucleic acid construct of claim 19 wherein the human GPCR gene is HTR1A or HTR7.

21. A non-human animal comprising the nucleic acid construct of claim 19 or 20 incorporated into its genome.

22. A method for screening human therapeutic agents that target one or more G protein-coupled receptors (GPCR), the method comprising:
a) inserting a nucleic acid construct of any one of claims 19-21into the genome of the non-human animal;
b) randomly mutagenizing the native promoter to create downward attenuated expression of the human GPCR gene and a corresponding loss-of-function (LOF) phenotype in the non-human animal; and,
c) treating the non-human animal with one or more GPCR agonists to identify a therapeutic agent that restores the LOF phenotype.

23. A nucleic acid construct comprising a promoter regulatory sequence for ubiquitous expression in C. elegans neurons or in zebrafish, a human cannabinoid receptor optimized for expression in C. elegans, and a 3′ untranslated region (UTR).

24. The nucleic acid construct of claim 23 wherein the human cannabinoid receptor is selected from the group consisting of CNR1, SEQ ID NO: 12, CNR2, and SEQ ID NO: 14.

25. A C. elegans animal or zebrafish comprising the nucleic acid construct of claim 23 or 24 incorporated into its genome.

26. The C. elegans animal or zebrafish of claim 25, wherein the C. elegans animal or zebrafish does not express at least one native endocannabinoid synthesis gene product, is depleted in or over-expresses the fatty acid amide hydrolase (faah-2) or ortholog thereof, and/or expresses at least one human clinical variant.

27. A method for identifying a therapeutic agent for cannabinoid treatment, the method comprising exposing a C. elegans animal or zebrafish of claim 25 or 26 to a potential therapeutic agent and detecting phenotypic changes in the C. elegans animal or zebrafish.

28. A C. elegans animal comprising a nucleic acid sequence encoding the coding sequence for human gamma-aminobutyric acid transaminase (hGABAT) in its genome.

29. The C. elegans animal of claim 28 wherein the nucleic acid sequence is SEQ ID NO: 16 or encodes a gene provided in Table 6.

30. The C. elegans animal of claim 28 or 29, further comprising within its genome at least one additional nucleic acid sequence encoding at least one additional human gene.

31. The C. elegans animal of claim 30 wherein the at least one additional human gene is hSTXBP1.

32. A method for identifying a therapeutic agent for cannabinoid treatment, the method comprising exposing a C. elegans animal of any one of claims 28-31to a potential therapeutic agent and detecting phenotypic changes in the C. elegans animal.

33. A C. elegans animal or zebrafish comprising a nucleic acid sequence encoding the coding sequence for a genetic variant associated with Alzheimer’s Disease in its genome.

34. The C. elegans animal or zebrafish of claim 33 wherein the nucleic acid sequence comprises a coding sequence of a gene selected from the group consisting of MAPT, GRN, TARDBP, APP, PSEN1, and PSEN2.

35. The C. elegans animal or zebrafish of claim 33 or 34 wherein the nucleic acid sequence is operably linked to at least one reporter sequence, optionally wherein the reporter is operably linked to a tissue-specific promoter.

36. The C. elegans animal or zebrafish of claim 35 wherein the tissue-specific promoter is specific for neurons.

37. The C. elegans animal or zebrafish of claim 35 or 36 wherein the reporter sequence is a fluorescent protein or a genetically encoded calcium indicators (GECI).

38. A method for identifying a therapeutic agent for Alzheimer’s Disease, the method comprising exposing a C. elegans animal or zebrafish of any one of claims 33-37to a potential therapeutic agent for Alzheimer’s Disease and detecting phenotypic changes in the C. elegans animal or zebrafish.

39. A C. elegans animal or zebrafish comprising a nucleic acid sequence encoding a human viral receptor in its genome.

40. The C. elegans animal or zebrafish of claim 39 wherein the nucleic acid sequence comprises a coding sequence of ACE2, optionally wherein the nucleic acid is optimized for expression therein.

41. The C. elegans animal or zebrafish of claim 39 or 40 wherein the nucleic acid sequence is operably linked to at least one reporter sequence, optionally wherein the reporter is a fluorescent reporter.

42. A method for identifying a therapeutic agent for an infectious disease, the method comprising exposing a C. elegans animal or zebrafish of any one of claims 39-41to a potential therapeutic agent for the infectious disease and detecting phenotypic changes in the C. elegans animal or zebrafish.


SEQUENCE LISTING (19 sequences)

SEQ ID NO: 1 - DNA, 1830 bp, C. elegans
atggctagcagcgctgagctacatcaaggggaacgtgaaacatggggaaagaaagtcgacttcctcctctccgtcatcggatacgccgtcgacctcggaaacgtctggcgtttcccatacatctgctaccaaaacggaggaggagccttcctcctcccatacaccatcatggccatcttcggaggaatcccactcttctacatggagctcgccctcggacaataccaccgtaacggatgcatctccatctggcgtaagatctgcccaatcttcaagggaatcggatacgccatctgcatcatcgccttctacatcgcctcttactacaacaccatcatggcctgggccctctactacctcatctcctccttcaccgaccaactcccatggacctcctgcaaggtacttgagatccttaaacgcagtcgaaaattggtaattttacagaactcctggaacaccggaaactgcaccaactacttctccgaggacaacatcacctggaccctccactccacctccccagccgaggagttctacacccgtcacgtcctccaaatccaccgttccaagggactccaagacctcggaggaatctcctggcaactcgccctctgcatcatgctcatcttcaccgtcatctacttctccatctggaagggagtcaagacctccggaaaggtcgtctgggtcaccgccaccttcccatacatcatcctctccgtcctcctcgtccgtggagccaccctcccaggagcctggcgtggagtcctcttctacctcaagccaaactggcaaaagctcctcgagaccggagtctggatcgacgccgccgcccaaatcttcttctccctcggaccaggattcggagtcctcctcgccttcgcctcctacaacaagttcaacaacaactgctaccaagacgccctcgtcacctccgtcgtcaactgcatgacctccttcgtctccggattcgtcatcttcaccgtcctcggatacatggccgagatgcgtaacgaggacgtctccgaggtcgccaaggtaagttcctccactagaaatatcaggtgctataattgtgttcaggacgccggaccatccctcctcttcatcacctacgccgaggccatcgccaacatgccagcctccaccttttttgccatcattttcttcctcatgcttataaccctcggactcgactccaccttcgccggactcgagggagtcatcactgccgtcctcgacgagttcccacacgtctgggccaagcgtcgtgagcgtttcgtcctcgccgtcgtcatcacctgcttcttcggatccctcgtcaccctcaccttcggaggagcctacgtcgtcaagctcctcgaggagtacgccaccggaccagccgtcctcaccgtcgccctcatcgaggccgtcgccgtctcctggttctacggaatcacccaattctgccgtgacgtcaaggtgagttattataatttttttgatcacaacgattattttaattttcaggagatgctcggattctccccaggatggttctggcgtatctgctgggtcgccatctccccactcttcctcctcttcatcatctgctccttcctcatgtccccaccacaactccgtctcttccaatacaactacccatactggtccatcatcctcggatactgcatcggaacctcctccttcatctgcatcccaacctacatcgcctaccgtctcatcatcaccccaggaaccttcaaggagcgtatcatcaagtccatcaccccagagaccccaaccgagatcccatgcggagacatccgtctcaacgccgtctaa

SEQ ID NO: 2 - DNA, 45 bp, C. elegans
gtacttgagatccttaaacgcagtcgaaaattggtaattttacag

SEQ ID NO: 3 - DNA, 45 bp, C. elegans
gtaagttcctccactagaaatatcaggtgctataattgtgttcag

SEQ ID NO: 4 - DNA, 48 bp, C. elegans
gtgagttattataatttttttgatcacaacgattattttaattttcag

SEQ ID NO: 5 - DNA, 2799 bp, C. elegans
atgctaacaaccggtggatcgggtggatcgatggtacaaaagtccagaaatggtggagtttacccgggtccatctggtgaaaaaaaattgaaagtaggatttgtcggcctcgaccctggagcgccggacagtaccagagatggcgcgctgttgatcgctggttcggaggcaccgaaacgaggaagtattctcagtaagcctcgtgcgggaggtgccggcgctggaaaaccgcctaaaagaaatgccttttacagaaagctgcagaacttcttgtataatgtgctggaacgaccgagaggctgggcatttatttatcacgcctacgttttcttgcttgttttctcctgccttgtgttgagtgttttctccaccataaaagaatacgaaaaaagttccgagggtgctctttacatcctcgaaattgtcaccatcgtggtgttcggagtggaatactttgttagaatttgggccgctggctgctgctgccgataccgaggctggcgaggtcgtctgaaatttgctcgaaaaccgttctgtgtcatcgacattatggttctgatcgcaagtattgctgtcttggcggcgggatctcagggcaatgtgtttgcaacctcggcccttagatccctccgatttttacaaatcctccgtatgatccgtatggaccgacgtggtggaacttggaaacttcttggatccgtcgtctacgcccactccaaggtgagtgattttaaacattatctgtacttaaattataaattctctattcaggaactcgtcaccgcctggtacatcggattcttgtgtcttatcctggcatcgtttcttgtttacttggccgaaaagggtgaaaacgatcactttgacacatatgccgatgcgttgtggtggggcttgatcactcttacgacaattggatatggtgacaagtatccgcagacatggaatggtagacttcttgctgccaccttcaccctgatcggtgtcagtttcttcgccctcccagctggcatcctgggctcaggttttgcgctgaaggtccaagagcagcaccgacaaaaacactttgaaaagcgacgtaaccctgccgctggtttgattcaatccgcttggagattctacgctacgaacttgtctcgtaccgatctgcactctacctggcaatactacgaaagaacggtaacagtgccgatgtattcgtcccaaactcaaacttacggagcttcaagactgattccaccgctgaaccagctggagctgttgcgaaaccttaaatcaaaatctggcctggctttccgaaaggatcctcctccggagccttcgccttctaagggaagtccttgcagaggcccgctttgcggttgctgcccaggacgttcctcccaaaaggtaaataattatacattcgatgataaatttatgcgtactatttttcaggtctccctcaaggaccgtgtcttctcctccccgagaggcgtagcagccaagggaaagggaagtccacaagcacaaactgttcgaagatcgccttcagcggaccaatcattggaagactcgccatcaaaggtgcctaaatcctggtcctttggtgaccgttcgagagcaagacaggccttccgtatcaagggtgcggcatctcgacagaattcggaagaagcttcactcccaggcgaggacatcgtggacgacaaatcttgtccgtgtgaatttgtgaccgaagacctcactccgggtttgaaagtgtctatcagagcggtgtgcgtgatgagattcctcgtctccaagcgtaaattcaaggaatccttgcgaccgtatgacgttatggacgttatcgaacaatactcagctggacatttggatatgctttcgcgtatcaagtccctccaaagtagagtggaccaaattgttggcagaggacctgcaatcaccgacaaggacagaacgaagggtcctgcggaagccgagctgcctgaggacccatcaatgatgggtagattgggcaaggttgaaaaacaagttttgagtatggagaagaaactggactttcttgtcaatatctatatgcaaagaatgggaatccctcctacggagaccgaggcctacttcggagccaaggttaaatgtacaaacaactatttgaaagattttctcacccgattttttcaggagcccgagccagcccctccataccactcaccagaagactcacgtgaacacgttgacagacacggttgcattgtgaaaattgttcgttcttcgtcctcgacgggtcagaaaaacttctcagcaccacctgctgcccctcctgtccaatgccctccgtcaactagttggcaaccgcaaagtcatccgcgtcagggccatggtacgagtccagtaggcgatcacggctcgttggtgcgaatcccgcctcctcctgcccacgagagatcattgtctgcctacggtggcggcaatcgagcatctatggagttcctgagacaagaagacaccccaggatgcagaccgccagagggtaaccttcgtgactctgacacgtccatttcaatcccttcagttgaccacgaagaactcgagagatccttcagtggattttccatctctcaatctaaagaaaatctggatgccctcaactcatgttatgcggcggtcgcaccgtgtgcaaaggttcgtccttacatcgcggagggagagagtgacacagacagtgacctgtgcacgccttgcggaccgccgccacgatcagctaccggagaaggccctttcggtgatgtgggatgggcaggccctcgaaaataa

SEQ ID NO: 6 - DNA, 20 bp, C. elegans
ttttgcatccgaaaaagcgg

SEQ ID NO: 7 - DNA, 501 bp, C. elegans
cttatcatttcaatttatttattaatcgatgattgaaagtgaatggatgacggtcatgaccgattatcgattatcccgaaatagagatgcgcgtaggtcataatgcccagtacgcaaaatgttttatcggtgtttgcacagatttcgcaacatctctcattgaatttccattcatcgcttcgtcatctgaccccatttcttattttttcatccttttccctgttctcatcgttccttactattttcctaatttcagaacatcgcgattttataatttcgttaaatattcgtaatcccgttatacaaaaatagctaaattttctagtcgttctcgtttttgagagggcactttagtccgtcatcgtgtcgcttgtcgtgctcaatttttcatgcataaatgggcgtcgccgtcccccctgtcgttttcttcctttacctcactttccagttctgaattccgatacgaatttttaaatttttctaactcgcttcatttcaggg

SEQ ID NO: 8 - DNA, 20 bp, C. elegans
ttttgcatccgaaaaagcgg

SEQ ID NO: 9 - DNA, 648 bp, C. elegans
cgattactaacataatatacctatttctgagcccaacgtccgctgctttcgggcgcacaaaagatgtaatccgagatctgcgatttgtacatatttcggatacattttcaatctccatttccaactcatcttctaattcgggacaatgagcatcatcatgagcatgtgtacacaaggtagttcggtcgcccagttgccgattacataggcacccggttgccgagtagacgagttgtaaaaacagggcaccacgagatattgccgatttccacataaactacttattaaagtctggagatttaattcatatgtcaagaaaaatgttcgtgaaagcaacaatatatgtactagaaatccctaagacgacgactatcgctgacgttacattgctccaaaagaatgcaatgaattgtgattacaccatgagagttttcaaaactttaaacctcaaattaatatttcaacttattaacctacttcctattcttttcaattctttatttgtctatcgtctctttattcccaatgatctctcttctaagagcttctaccttatcggctactgtgtccaattacccatcgacgctcctccctcttttgcgcgaaactgggcggagcctaagatgagattcagtaaaataggttctc

SEQ ID NO: 10 - DNA, 81 bp, C. elegans
gggctacgtaatacgactcacttaaggccttaatcccaataaacctgtattcctgtgtgtcgtcaagatggtcttatccgg

SEQ ID NO: 11 - DNA, 61 bp, C. elegans
gccctctaagataaatgcaaaatcctttcaagcattccctgagacttttttcttggcggca

SEQ ID NO: 12 - DNA, 1569 bp, C. elegans
atgaaatcgatcttggatggacttgcagatactacgttccgtaccatcaccaccgacctcctctacgtcggatccaacgacatccaatacgaggacatcaagggagacatggcctccaagctcggatacttcccacaaaagttcccactcacctccttccgtggatccccattccaagagaagatgaccgccggagacaacccacaactcgtcccagccgaccaagtcaacatcaccgagttctataataagtccctctcctccttcaaggtaagtttaaacatatatatactaactaaccctgattatttaaattttcaggagaatgaggagaacatccaatgcggagagaacttcatggacatcgagtgcttcatggtcctcaacccatcccaacaactcgccatcgccgtcctctccctcaccctcggaaccttcaccgtcctcgagaacctcctcgtcctctgcgtcatcctccactcccgttccctccgttgccgtccatcctaccacttcatcggatccctcgccgtcgccgacctcctcggatccgtcatcttcgtctactccttcatcgacttccacgtcttccacagaaaggactcccgtaacgtcttcctctttaagcttggaggagttactgcctccttcaccgcttccgtcggatccctcttcctcaccgccatcgaccgttacatctccatccaccgtccactcgcctacaagcgtatcgtcacccgtccaaaggtaagtttaaaagttcgtactaactaaccatacatatttaaattttcaggccgtcgtcgccttctgcctcatgtggaccatcgccatcgtcatcgccgtcctcccactcctcggatggaactgcgagaagctccaatccgtctgctccgacatcttcccacacatcgacgagacctacctcatgttctggatcggagtcacctccgtcctcctcctcttcatcgtctacgcctacatgtacatcctctggaaggcccactcccacgccgtccgtatgatccaacgtggaacccaaaagtccatcatcatccacacctccgaggacggaaaggtccaagtcacccgtccagaccaagctagaatggacatccgtctcgccaaggtaagtttaacatgattttactaactaactaatctgatttaaattttcagaccctcgtcctcatcctcgtcgtcctcatcatctgctggggaccactcctcgccatcatggtctacgacgtcttcggaaagatgaacaagctcatcaagaccgtcttcgccttctgctccatgctctgcctcctcaactccaccgtcaacccaatcatctacgccctccgttccaaggacctccgtcacgccttccgttccatgttcccatcctgcgagggaaccgcccaaccactcgacaactccatgggagactccgactgcctccacaagcacgccaacaacgccgcctccgtccaccgtgccgccgagtcctgcatcaagtccaccgtcaagatcgccaaggtcaccatgtccgtctccaccgacacctccgccgaggccctctaa

SEQ ID NO: 13 - protein, 472 aa, C. elegans
MKSILDGLADTTFRTITTDLLYVGSNDIQYEDIKGDMASKLGYFPQKFPLTSFRGSPFQEKMTAGDNPQLVPADQVNITEFYNKSLSSFKENEENIQCGENFMDIECFMVLNPSQQLAIAVLSLTLGTFTVLENLLVLCVILHSRSLRCRPSYHFIGSLAVADLLGSVIFVYSFIDFHVFHRKDSRNVFLFKLGGVTASFTASVGSLFLTAIDRYISIHRPLAYKRIVTRPKAVVAFCLMWTIAIVIAVLPLLGWNCEKLQSVCSDIFPHIDETYLMFWIGVTSVLLLFIVYAYMYILWKAHSHAVRMIQRGTQKSIIIHTSEDGKVQVTRPDQARMDIRLAKTLVLILVVLIICWGPLLAIMVYDVFGKMNKLIKTVFAFCSMLCLLNSTVNPIIYALRSKDLRHAFRSMFPSCEGTAQPLDNSMGDSDCLHKHANNAASVHRAAESCIKSTVKIAKVTMSVSTDTSAEAL

SEQ ID NO: 14 - DNA, 1227 bp, C. elegans
atggaggaatgttgggttacagagatagcaaatggtagcaaggacggactcgattccaaccctatgaaagactacatgatcctctccggaccacaaaagaccgccgttgccgttctctgcaccctcctcggactcctctctgctcttgagaacgtcgccgtcctctacctcatcctctcctcccaccaactccgtcgtaagccatcctacctcttcatcggatccctcgccggagccgacttcctcgcctccgtcgtcttcgcctgctccttcgtcaacttccacgtcttccacggagtcgactccaaggtaagtttaaacatatatatactaactaaccctgattataaattttcaggccgtcttcctcctcaagatcggatccgtcaccatgaccttcaccgcctccgtcggatccctcctcctcaccgccatcgaccgttacctctgcctccgttacccaccatcctacaaggccctcctcacccgtggacgtgccctcgtcaccctcggaatcatgtgggtcctctccgccctcgtctcctacctcccactcatgggatggacctgctgcccacgtccatgctccgagctcttcccactcatcccaaacgactacctcctctcctggctcctcttcatcgccttcctcttctccggaatcatctacacctacggacacgtcctctggaaggtaagttcctccactagaaatatcaggtgctataattgtgttcaggcccaccaacacgtcgcctccctctccggacaccaagaccgtcaagtcccaggaatggcccgtatgcgtctcgacgtccgtctcgccaagaccctcggactcgtcctcgccgtcctcctcatctgctggttcccagtcctcgccctcatggcccactccctcgccaccaccctctccgaccaagtcaaggtaagtttaacatgattttactaactaactaatctgatttaaattttcagaaggccttcgccttctgctccatgctctgcctcatcaactccatggtcaacccagtcatctacgccctccgttccggagagatccgttcctccgcccaccactgcctcgcccactggaagaagtgcgtccgtggactcggatccgaggccaaggaggaggccccacgttcctccgtcaccgagaccgaggccgacggaaagatcaccccatggccagactcccgtgacctcgacctctccgactgctaa

SEQ ID NO: 15 - protein, 360 aa, C. elegans
MEECWVTEIANGSKDGLDSNPMKDYMILSGPQKTAVAVLCTLLGLLSALENVAVLYLILSSHQLRRKPSYLFIGSLAGADFLASVVFACSFVNFHVFHGVDSKAVFLLKIGSVTMTFTASVGSLLLTAIDRYLCLRYPPSYKALLTRGRALVTLGIMWVLSALVSYLPLMGWTCCPRPCSELFPLIPNDYLLSWLLFIAFLFSGIIYTYGHVLWKAHQHVASLSGHQDRQVPGMARMRLDVRLAKTLGLVLAVLLICWFPVLALMAHSLATTLSDQVKKAFAFCSMLCLINSMVNPVIYALRSGEIRSSAHHCLAHWKKCVRGLGSEAKEEAPRSSVTETEADGKITPWPDSRDLDLSDC

SEQ ID NO: 16 - DNA, 1641 bp, C. elegans
atggcctccatgctcctcgcccaacgtctcgcctgctccttccaacactcctaccgtctcctcgtcccaggatcccgtcacatctcccaagccgccgccaaggtcgacgtcgagttcgactacgacggaccactcatgaagaccgaggtcccaggaccacgttcccaagagctcatgaagcaactcaacatcatccaaaacgccgaggccgtccacttcttctgcaactacgaggagtcccgtggaaactacctcgtcgacgtcgacggaaaccgtatgctcgacctctactcccaaatctcctccgtcccaatcggatactcccacccagccctcctcaagctcatccaacaaccacaaaacgcctccatgttcgtcaaccgtccagccctcggaatcctcccaccagagaacttcgtcgagaagctccgtcaatccctcctctccgtcgccccaaaggtacttgagatccttaaacgcagtcgaaaattggtaattttacagggaatgtcccaactcatcaccatggcctgcggatcctgctccaacgagaacgccctcaagaccatcttcatgtggtaccgttccaaggagcgtggacaacgtggattctcccaagaggagctcgagacctgcatgatcaaccaagccccaggatgcccagactactccatcctctccttcatgggagccttccacggacgtaccatgggatgcctcgccaccacccactccaaggccatccacaagatcgacatcccatccttcgactggccaatcgccccattcccacgtctcaagtacccactcgaggagttcgtcaaggtaagttcctccactagaaatatcaggtgctataattgtgttcaggagaaccaacaagaggaggcccgttgcctcgaggaggtcgaggacctcatcgtcaagtaccgtaagaagaagaagaccgtcgccggaatcatcgtcgagccaatccaatccgagggaggagacaaccacgcctccgacgacttcttccgtaagctccgtgacatcgcccgtaagcacggatgcgccttcctcgtcgacgaggtccaaaccggaggaggatgcaccggaaagttctgggcccacgagcactggggactcgacgacccagccgacgtcatgaccttctccaagaagatgatgaccggaggattcttccacaaggtgagttattataatttttttgatcacaacgattattttaattttcaggaggagttccgtccaaacgccccataccgtatcttcaacacctggctcggagacccatccaagaacctcctcctcgccgaggtcatcaacatcatcaagcgtgaggacctcctcaacaacgccgcccacgccggaaaggccctcctcaccggactcctcgacctccaagcccgttacccacaattcatctcccgtgtccgtggacgtggaaccttctgctccttcgacaccccagacgactccatccgtaacaagctcatcctcatcgcccgtaacaagggagtcgtcctcggaggatgcggagacaagtccatccgtttccgtccaaccctcgtcttccgtgaccaccacgcccacctcttcctcaacatcttctccgacatcctcgccgacttcaagtaa

SEQ ID NO: 17 - DNA, 20 bp, zebrafish
tagtggaccagctcagcatg

SEQ ID NO: 18 - DNA, 20 bp, zebrafish
gatatcagtcattttgcagc

SEQ ID NO: 19 - DNA, 50 bp, zebrafish
cggtgcgggtgccagggcgtgcccttgggctccccgggcgcgtactccac